SEQUENCE LISTING (28 sequences)

SEQ ID NO: 1 - DNA, 20 bp, synthetic construct
  misc_feature 1..20: note = Synthetic Polynucleotide
  source 1..20: mol_type = other DNA
aagtcaacttcaatgtcgga

SEQ ID NO: 2 - DNA, 20 bp, synthetic construct
  misc_feature 1..20: note = Synthetic Polynucleotide
  source 1..20: mol_type = other DNA
cgtgagtaaacctgaatctt

SEQ ID NO: 3 - DNA, 20 bp, synthetic construct
  misc_feature 1..20: note = Synthetic Polynucleotide
  source 1..20: mol_type = other DNA
actcacgctggatagcctcc

SEQ ID NO: 4 - DNA, 20 bp, synthetic construct
  misc_feature 1..20: note = Synthetic Polynucleotide
  source 1..20: mol_type = other DNA
gagtagcgcgagcacagcta

SEQ ID NO: 5 - DNA, 20 bp, synthetic construct
  misc_feature 1..20: note = Synthetic Polynucleotide
  source 1..20: mol_type = other DNA
gtagatacaactgaccctgt

SEQ ID NO: 6 - DNA, 20 bp, synthetic construct
  misc_feature 1..20: note = Synthetic Polynucleotide
  source 1..20: mol_type = other DNA
tactagcatggtgctcacca

SEQ ID NO: 7 - DNA, 20 bp, synthetic construct
  misc_feature 1..20: note = Synthetic Polynucleotide
  source 1..20: mol_type = other DNA
caggacctccacatgatagg

SEQ ID NO: 8 - DNA, 20 bp, synthetic construct
  misc_feature 1..20: note = Synthetic Polynucleotide
  source 1..20: mol_type = other DNA
tgcagtactcggacgagttc

SEQ ID NO: 9 - DNA, 20 bp, synthetic construct
  misc_feature 1..20: note = Synthetic Polynucleotide
  source 1..20: mol_type = other DNA
ttataaggatgtcatccccg

SEQ ID NO: 10 - DNA, 20 bp, synthetic construct
  misc_feature 1..20: note = Synthetic Polynucleotide
  source 1..20: mol_type = other DNA
ggtggtgcagatgaacttca

SEQ ID NO: 11 - DNA, 20 bp, synthetic construct
  misc_feature 1..20: note = Synthetic Polynucleotide
  source 1..20: mol_type = other DNA
ggagcgcaccatcttcttca

SEQ ID NO: 12 - DNA, 20 bp, synthetic construct
  misc_feature 1..20: note = Synthetic Polynucleotide
  source 1..20: mol_type = other DNA
ggcatcgacttcaaggagga

SEQ ID NO: 13 - DNA, 20 bp, synthetic construct
  misc_feature 1..20: note = Synthetic Polynucleotide
  source 1..20: mol_type = other DNA
aaacgcctaagcctagttgt

SEQ ID NO: 14 - DNA, 20 bp, synthetic construct
  misc_feature 1..20: note = Synthetic Polynucleotide
  source 1..20: mol_type = other DNA
tgtgcagtccacgatcacat

SEQ ID NO: 15 - DNA, 20 bp, synthetic construct
  misc_feature 1..20: note = Synthetic Polynucleotide
  source 1..20: mol_type = other DNA
atcgagggcaatttccactt

SEQ ID NO: 16 - DNA, 20 bp, synthetic construct
  misc_feature 1..20: note = Synthetic Polynucleotide
  source 1..20: mol_type = other DNA
tctggtgtgtcaatgggacg

SEQ ID NO: 17 - DNA, 20 bp, synthetic construct
  misc_feature 1..20: note = Synthetic Polynucleotide
  source 1..20: mol_type = other DNA
caagctatgtaacacaccgc

SEQ ID NO: 18 - DNA, 20 bp, synthetic construct
  misc_feature 1..20: note = Synthetic Polynucleotide
  source 1..20: mol_type = other DNA
cccacgttaagcgcgaccaa

SEQ ID NO: 19 - DNA, 20 bp, synthetic construct
  misc_feature 1..20: note = Synthetic Polynucleotide
  source 1..20: mol_type = other DNA
gtctgcgggagacccccgcg

SEQ ID NO: 20 - DNA, 20 bp, synthetic construct
  misc_feature 1..20: note = Synthetic Polynucleotide
  source 1..20: mol_type = other DNA
cagtagtccaagttcgtgcg

SEQ ID NO: 21 - DNA, 20 bp, synthetic construct
  misc_feature 1..20: note = Synthetic Polynucleotide
  source 1..20: mol_type = other DNA
cacctagcctcgcacgaact

SEQ ID NO: 22 - DNA, 20 bp, synthetic construct
  misc_feature 1..20: note = Synthetic Polynucleotide
  source 1..20: mol_type = other DNA
gcatgcgagaatctcacgca

SEQ ID NO: 23 - DNA, 20 bp, synthetic construct
  misc_feature 1..20: note = Synthetic Polynucleotide
  source 1..20: mol_type = other DNA
gtagatacaactgaccctgt

SEQ ID NO: 24 - DNA, 20 bp, synthetic construct
  misc_feature 1..20: note = Synthetic Polynucleotide
  source 1..20: mol_type = other DNA
caggaagcagtgaccaaagg

SEQ ID NO: 25 - DNA, 20 bp, synthetic construct
  misc_feature 1..20: note = Synthetic Polynucleotide
  source 1..20: mol_type = other DNA
tttggatggtgctggattgg

SEQ ID NO: 26 - DNA, 20 bp, synthetic construct
  misc_feature 1..20: note = Synthetic Polynucleotide
  source 1..20: mol_type = other DNA
tactagcatggtgctcacca

SEQ ID NO: 27 - DNA, 20 bp, synthetic construct
  misc_feature 1..20: note = Synthetic Polynucleotide
  source 1..20: mol_type = other DNA
gctgcagagggtaaacaggg

SEQ ID NO: 28 - DNA, 20 bp, synthetic construct
  misc_feature 1..20: note = Synthetic Polynucleotide
  source 1..20: mol_type = other DNA
tcctggagaacatggtgtgg

(19) United States
(12) Patent Application Publication
(10) Pub. No.: US 2023/0295665 A1
(43) Pub. Date: Sep. 21, 2023

(54) EFFICIENT GENOME EDITING IN PRIMARY MYELOID CELLS

(71) Applicant: Genentech, Inc., South San Francisco, CA (US)

(72) Inventors: Aditya Krishna MURTHY, South San Francisco, CA (US); Jaehak OH, South San Francisco, CA (US); Christopher John BOHLEN, South San Francisco, CA (US); Emily Crane FREUND, South San Francisco, CA (US); Benjamin Joseph HALEY, South San Francisco, CA (US); Jaclyn LOCK, South San Francisco, CA (US)

(21) Appl. No.: 17/822,746

(22) Filed: Aug. 26, 2022

Related U.S. Application Data
(63) Continuation of application No. PCT/US2021/019488, filed on Feb. 24, 2021.
(60) Provisional application No. 63/010,476, filed on Apr. 15, 2020.
(60) Provisional application No. 62/983,568, filed on Feb. 28, 2020.

(51) Int. Cl. C12N 15/90; C12N 9/22; C12N 15/11; G01N 33/50; A61K 35/15

(52) U.S. Cl. CPC C12N 15/907; A61K 35/15; C12N 9/22; C12N 15/11; G01N 33/5047; C12N 2310/20; C12N 2800/80

(57) ABSTRACT

Provided herein, inter alia, are compositions, methods, and systems for efficient genetic manipulation of myelod cells without the use of viral vectors. Further provided are strategies for gene disruption in primary myeloid cells (e.g. of human and murine origin) using electroporation-based delivery of Cas/ribonuclear proteins (RNPs). Methods provided herein including embodiments thereof can provide near population-level genetic knockout of single and multiple targets in a range of cell types without selection or enrichment. Cellular fitness and response to immunological stimuli may be unaffected by the gene editing process.

### CROSS-REFERENCES TO RELATED APPLICATIONS

This application claims the benefit of U.S. Provisional Application No. 62/983,568, filed Feb. 28, 2020 and U.S. Provisional Application No. 63/010,476, filed Apr. 15, 2020, each of which is incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII FILE

The Sequence Listing written in file 048893-530001WO_ST25, created on Feb. 23, 2021, 5,790 bytes, machine format IBM-PC, MS-Windows operating system, is hereby incorporated by reference.

### BACKGROUND

Myeloid cells play critical and diverse roles in mammalian physiology, including tissue development and repair, innate defense against pathogens and generation of adaptive immunity. Macrophages and dendritic cells, in particular, are therapeutic targets for a number of diseases, including cancer. However, few approaches have been developed for gene editing of these cell types, likely due to their sensitivity to foreign genetic material or virus-based manipulation.

Myeloid cells constitute the innate immune system, providing a first line of defense against pathogens while also generating the requisite inflammation for optimal adaptive immunity. Myeloid cell subsets include granulocytes, macrophages, monocytes and dendritic cells. These cells are critical components of the tissue microenvironment, acting as effectors for direct killing of pathogens or infected cells, as phagocytes to clear dead cells or pathogens, as professional antigen presenting cells (APCs) to drive adaptive immunity and finally as modifiers of the microenvironment by generation of inflammatory or reparative factors such as cytokines. Strategies targeting myeloid cells have emerged as relevant for promoting anti-microbial or antitumor immunity, making research into the biology or modification of these cell types necessary for advancing immunomodulatory therapeutics. In spite of this, current research on innate immunity is largely restricted to transformed myeloid cell lines, myeloid cells derived from Cas9-knock-in mouse models, or other engineered murine genetic models (e.g. knockout (KO), inducible cell deletion, reporter).

### SUMMARY

The instant disclosure generally relates to strategies for gene perturbation in primary myeloid cells, e.g. of human and murine origin. Using transfection techniques (e.g., electroporation/nucleofection) to deliver ribonuclear protein complexes (RNPs) including guide-RNAs non-covalently associated with recombinant Cas (e.g. Cas9), near population-level genetic knockout of single and multiple targets can be achieved in a range of cell types without the need for selection or enrichment of genetically modified cells. Cellular fitness and response to immunological stimuli of cells modified according to the methods described herein are not significantly affected by the gene editing process. Such advances enable pathway discovery and drug target validation across species in the field of innate immunity.

Delivery via transfection, e.g., nucleofection of Cas-RNP complexes to non-granulocytic cells (i.e. monocytes, macrophages and dendritic cells) in accordance with the methods described herein can generate up to at least about 90% knockout of single or multiple target genes without the need further selection of genetically modified cells. This can be achieved in both differentiated primary myeloid cell populations and freshly isolated cells. Provided herein are methods that enable rapid loss-of-function gene assessment within donor cell populations without substantially affecting normal cell function, including, for example, responsiveness to stimulation.

In an aspect, provided herein are methods for genetic modification of a myeloid cell including transfecting the myeloid cell with a gene editing reagent targeting a genetic site of interest, where the myeloid cell is not transduced with a viral vector. In embodiments, a gene editing reagent suitable for use with the methods described herein can include an RNP including a guide RNA non-covalently contacted with a Cas protein, such as a Cas9 protein.

In an aspect, provided herein are methods for genetic modification of a plurality of myeloid cells, including transfecting the plurality of myeloid cells with a gene editing reagent targeting a site of interest, wherein the myeloid cells are not transduced with a viral vector or viral delivery system.

In an aspect, provided herein are methods of genetically modifying a plurality of myeloid cells, including transfecting the myeloid cells with a gene editing reagent, wherein the myeloid cells are not transduced with a viral vector, and wherein the method does not include a selection step or enrichment step following myeloid cell transfection

In an aspect, provided herein are systems for genetically modifying a myeloid cell in the absence of a viral vector. In embodiments, the systems include electroporation systems. In embodiments, the systems include nucleofection systems. In embodiments, the system includes a chamber compatible with electroporation or nucleofection system, multiple myeloid cells within the chamber in a media compatible with electroporation/nucleofection, and at least one gene editing system designed to target at least one site of interest in the genome of myeloid cells.

In an aspect, provided herein are methods of treating a disease treatable with a myeloid cell provided herein including embodiments thereof. The methods include providing a genetically modified myeloid cell that has not been transformed with a virus, where the myeloid cell has been transfected with a gene editing reagent; and administering the myeloid cell to a patient in need thereof.

In an aspect, provided herein is a genetically modified myeloid cell made by a method provided herein including embodiments thereof.

In an aspect, provided herein is an assay for drug discovery comprising screening the effect of one or more compounds on a genetically modified myeloid cell provided herein including embodiments thereof.

In an aspect, provided herein is a method for target validation of a compound, including contacting a genetically modified myeloid cell provided herein including embodiments thereof with the compound and monitoring an effect on the cell

In an aspect, provided herein are compositions including a plurality of myeloid cells in contact with a gene editing reagent, a transfection buffer, and an electroporation enhancer, where the composition does not comprise a viral vector.

### BRIEF DESCRIPTION OF THE DRAWINGS

FIGS. 1A-1E illustrate efficient gene editing in murine monocytes, macrophages and dendritic cells obtained from the bone marrow. (FIG. 1A) Workflow for screening crRNA/Cas9-RNP mediated knockout of eGFP in mouse monocytes. Representative FACS plots show gating strategy for identifying eGFP KO, F4/80+ macrophages following 5 days of culture in M-CSF. (FIG. 1B) Heatmaps depicting relative impact of nucleofection conditions on cell viability and eGFP knockout. White box indicates the best condition (Buffer P3, program CM-137). (FIG. 1C) eGFP KO efficiency following nucleofection with single or pooled crRNAs. NTC: Non-targeting control crRNA-Cas9-RNP. (FIG. 1D) Workflow for CD45 crRNAXT/Cas9-RNP mediated KO in mouse BMDC cultures. Top FACS plots (right side) show gating strategy for identifying macrophage, pDC, CD24+ DC, and Sirpα+ DC cells. Bottom FACS plots depict representative gating strategy using NTC crRNAXT/Cas9 RNP as a control for determining CD45 negative cells in each cell population. (FIG. 1E) CD45 KO efficiency as measured by FACS (experimental workflow and gating strategy shown in FIG. 1D) in BM derived CD24+, Sirpα+, pDC and macrophage cells nucleofected with IDT V3 Cas9-RNPs loaded with NTC or CD45 targeting crRNAXT using either P3, CM137 (top graph) or P3, EN138 (bottom graph) combinations. Data are presented as mean +/- SD and collected from two independent experiments.

FIGS. 2A-2G illustrate population-level gene editing in human monocyte derived dendritic cells and macrophages. (FIG. 2A) Workflow for β2-microglobulin (B2M) gRNA/Cas9-RNP mediated KO in human monocyte derived dendritic and macrophage cultures. Representative FACS plots show gating strategy for using Cas9-RNPs loaded with NTC gRNA to determine B2M negative cells in each cell population. (FIG. 2B) B2M KO efficiency in monocyte derived DCs (left bar graph) and macrophage (right bar graph) cells nucleofected with distinct B2M targeting sequences in either crRNA (B2M cr1, B2M cr2) or crRNAXT (B2M crXT1, B2M crXT2) format, or non-target controls (NTC) complexed with IDT V3 Cas9 (dark bars) or Thermo Fisher TruCut V2 Cas9 (light bars). Data are from one experiment. (FIG. 2C) B2M KO efficiency in monocyte-derived macrophages nucleofected with IDT V3 Cas9-RNPs loaded with two different crRNAXTs (crXT1, crXT2) targeting B2M or NTC (NTC crXT). Cas9-RNPs were added individually or in combination. Each Cas9-RNP is labeled with 1X or 2X to indicate the relative molar quantity nucleofected into the cells. (FIG. 2D) Same as in (FIG. 2B) but gRNA/Cas9-RNPs are loaded with sgRNAs (B2M sg1, sg2) instead of crRNAs or crRNAXTs. Data are from one experiment. (FIG. 2E) B2M KO efficiency in monocyte derived macrophages for IDT V3 Cas9-RNPs loaded with NTC sgRNA or B2M sgRNA2 and complexed with an sgRNA:Cas9 molar ratio of 2:1 or 3:1. Data are from one experiment. (FIG. 2F-FIG. 2G) Dose response curve of B2M KO efficiency in monocyte derived macrophages. IDT V3 Cas9-RNPs loaded with 2 different sgRNAs targeting B2M (FIG. 2F shows data for sg2, FIG. 2G shows data for sg4) were nucleofected into cells at the indicated quantities. Cas9-RNPs were complexed and delivered with and without 4 µM of IDT “electroporation enhancer”. Data are from one experiment. For (FIG. 2B-FIG. 2D) No Nucleofection (No Nuc) cells or cells nucleofected with NTC crRNA, crRNAXT or sgRNA/Cas9-RNPs were used as controls. Buffer P3, CM-137 condition was used for all Cas9-RNP delivery.

FIGS. 3A-3E illustrate efficient CRISPR/Cas9 deletion of Toll-like receptor 7 in murine BMDCs and MAVS in human monocyte derived dendritic cells. (FIG. 3A) Top: percent of cells that were TLR7 negative from BM derived CD24+, Sirpα+ DCs, pDCs and macrophages electroporated with IDT V3 Cas9-RNPs loaded with a NTC or 2 different TLR7 sgRNAs (sg1, sg2). TLR7 negative cells were assayed by intracellular FACS. Bottom: histograms depict the % max TLR7 for each BM derived cell subset electroporated with Cas9-RNPs loaded with NTC, TLR7 sg1, TLR7 sg2 or stained with isotype control. Data are represented as mean +/- SD. (FIG. 3B) Cytokine levels measured by Luminex in supernatant from BMDC culture (combined cell types) in (FIG. 3A) after stimulation with mock or 800 ng/ml of the TLR7 agonist R848 for 17 hrs. Data are represented as mean +/- SD and are from one experiment with 3 technical replicates. (FIG. 3C) Surface CD80 levels (gMFI) of each BMDC cell population in (FIG. 3A) after stimulation with mock or 800 ng/ml of the TLR7 agonist R848 for 17 hrs. Data are from one experiment with 3 technical replicates. Data are represented as mean +/- SD and are from one experiment with 3 technical replicates. (FIG. 3D) TIDE analysis of genomic DNA from monocyte derived dendritic cells electroporated with IDT V3 Cas9-RNPs loaded with two different sgRNAs against MAVS (sg1, sg2). ICE represents % indels and KO represents % knockout. (FIG. 3E) Cytokine levels measured by Luminex from the supernatant of the monocyte derived dendritic cells in (FIG. 3D) after stimulation with mock or the RIG-I agonist, 3P-dsRNA, overnight. Data represented as mean +/- SD. and are from one experiment with 3 technical replicates.

FIGS. 4A-4B illustrate single or combined deletion of MYD88, TRIF and STING in murine BMDMs impacts TLR and cytosolic sensing of microbial ligands. (FIG. 4A) Representative Western blots depicting single, double and triple gene knock-down by sgRNA/Cas9-RNP in murine BMDMs. (FIG. 4B) Cytokine measurements (ELISA) of IFNβ and TNF in cell culture supernatant following stimulation with the indicated ligand for 18 hours. Data are mean +/- SD (n=3) and representative of two independent experiments.

FIGS. 5A-5D illustrate screening of optimal Cas9-RNP electroporation protocols for KO in murine monocytes and BMDMs. (FIG. 5A) Ranking of nucleofection conditions for crRNA/Cas9-mediated eGFP KO (left graph) and viability (right graph) in monocyte-derived macrophages following 5 days of culture. Gray arrow: Buffer P3, Program CM-137. White arrow: Buffer P5, Program CM-150. Black arrow: Buffer P5, Program CA-137. Black bars: Nucleofection controls without crRNA/Cas9. (FIG. 5B) Workflow for generation of murine BMDMs and screening of crRNA/Cas9-mediated Itgam/CD1 1b KO. (FIG. 5C) Representative histograms depicting mean fluorescence intensity (MFI) of CD11b for indicated nucleofection conditions following 5 days of culture in BMDM media. (FIG. 5D) Heatmaps depicting relative impact of nucleofection conditions on cell viability and CD11b MFI. White box indicates the best condition (Buffer P3, program CM-137).

FIGS. 6A-6D illustrate screening of optimal Cas9-RNP nucleofection protocol for KO in murine BMDCs. (FIG. 6A) Workflow for an initial 80-condition (5 buffers, 15 electroporation programs, and 5 no nucleofection (No Nuc) controls) screen optimizing nucleofection parameters for efficient KO of CD45 through electroporation of IDT V3 including CD45 crRNAXT/Cas9-RNPs in murine BMDCs. Representative FACS plots showing the same gating strategy for identifying macrophage, pDC, CD24+ DC, and Sirpα+ DCs, and CD45 KO efficiency as shown in FIG. 1D. (FIG. 6B) Data from the initial optimization screen is shown in four heatmaps reporting the cell abundances and CD45 KO efficiency in CD24+ (left panels) and Sirpα+ (right panels) DCs. White boxes indicate the 5 conditions that showed the highest KO efficiency while maintaining acceptable cell abundance. Data are from one experiment. (FIG. 6C) Confirmation of deletion efficiency of the top five conditions from the initial optimization using IDT V3 including CD45 crRNAXT/Cas9-RNPs as compared to IDT V3 including NTC crRNAXT/Cas9-RNPs and No Nucleofection (NN) controls. Data are from one experiment. (FIG. 6D) Relative CD80 levels as measured by FACS in BM derived CD24+ DC, Sirpα+ DC, pDC and macrophages not nucleofected (No Nuc) or nucleofected with NTC crRNAXT/Cas9-RNPs (NTC crXT) using either P3, CM137 or P3, EN138 combinations. Data is presented as mean +/- SEM and collected from two independent experiments.

FIGS. 7A-7H illustrate supporting data for population-level gene disruption in human monocyte derived dendritic cells and macrophages. (FIG. 7A) B2M KO efficiency in monocyte derived macrophages nucleofected with 4 different crRNAXTs (crXT1, crXT2, crXT3, crXT4) targeted against B2M and complexed with IDT V3 Cas9. Data are from one experiment. (FIG. 7B) B2M KO efficiency in monocyte derived macrophages electroporated with 2 different crRNAXTs (crXT1, crXT2) targeted against B2M and complexed with IDT V3 Cas9. Data from three different donors are displayed. (FIG. 7C) B2M KO efficiency in monocyte derived macrophages nucleofected with indicated crRNAs targeted against B2M or non-targeting control (NTC) and complexed with IDT V3 Cas9 (n=3). (FIG. 7D) B2M KO efficiency in monocyte derived macrophages nucleofected with a single sgRNA (sg2) and complexed with IDT V3 Cas9. Data are from three different donors with 3 technical replicates and are displayed as mean +/- S.D. (FIG. 7E) B2M KO efficiency in monocyte derived macrophages nucleofected with IDT V3 including Cas9-RNPs loaded with two different sgRNAs (sg1, sg2) targeting B2M or a non-targeting control sgRNA (NTC sg). Cas9-RNPs 1021 were added individually or in combination. Each Cas9-RNP is labeled with 1x or 2x to indicate the relative molar quantity nucleofected into the cells. Data are mean +/- S.D. (n=3). (FIG. 7F) B2M KO efficiency in monocyte derived macrophages for IDT V3 Cas9-RNPs loaded with NTC sgRNA or B2M sgRNA2 and complexed with an sgRNA:Cas9 molar ratio of 2:1 or 3:1. (FIG. 7G-FIG. 7H) Cell viability in monocyte derived macrophages from samples in FIGS. 2E and 2F. FIG. 7G shows data for sg2 and FIG. 7H shows data for sg4.

FIGS. 8A-8D illustrate supporting data for CRISPR/Cas9 deletion of MAVS and PKR in human monocyte macrophages and dendritic cells. (FIG. 8A) MAVS KO efficiency as determined by intracellular FACS staining in monocyte derived dendritic cells electroporated with 2 different sgRNAs (sg1, sg2) targeted against MAVS and complexed with IDT V3 Cas9. Data are from one experiment with 3 technical replicates and presented as mean +/- SD (FIG. 8B) Sanger sequencing traces used to determine TIDE values in FIG. 3D. Underlined nucleotides in control samples (electroporated with NTC sg) indicate sgRNA targeting sequences. T1 and T2 represent technical replicates. (FIG. 8C) Workflow for PKR sgRNA/Cas9-RNP mediated KO in human monocyte derived macrophages followed by stimulation with PKR activator Poly I:C or mock. (FIG. 8D) Western blot of cell lysates from PKR sgRNA/Cas9-RNP electroporated human monocyte derived macrophages with or without stimulation with PKR activator Poly I:C. Blotted for total PKR, phosphorylated eIF2α, total eIF2α, or β-Tubulin as a loading control.

FIGS. 9A-9F illustrate supporting data for the disruption of single or multiple genes in murine BMDCs and BMDMs to study TLR signaling. (FIG. 9A) Percent of cells that were TLR7 negative from BM derived CD24+, Sirpα+ DCs, pDCs and macrophages nucleofected with IDT V3 Cas9-RNPs loaded with a NTC or 2 different Tlr7- specific sgRNAs (sg1, sg2). Top: Buffer P3, Program CM-137; Bottom: Buffer P3, Program EN-138. TLR7 negative cells were assayed by intracellular FACS. Data are represented as mean +/- S.D.. (FIG. 9B) Cytokine levels measured by Luminex in supernatant from BMDC culture (combined cell types) in (FIG. A) after stimulation with mock or 800 ng/ml of the TLR7 agonist R848 for 17 hrs. Data are represented as mean +/-S.D. and are from one experiment with 3 technical replicates. (FIG. 9C) Representative western blots depicting MyD88 or TRIF knock-down by sgRNA-Cas9-RNP in murine BMDMs. (FIG. 9D) Assessment of gene editing efficiency by Sanger sequencing 5 days after electroporation. Data are mean +/- S.E.M. (n=3). (FIG. 9E) ELISA measurement of IFNβ levels in cell culture media of BMDMs 24 hours after electroporation and 5 days after electroporation treated as described. (FIG. 9F) ELISA measurements of TNF in cell culture supernatant following stimulation with the indicated ligand for 18 hours. Data in FIGS. 9E and 9F are mean +/- S.D. (n=3) and representative of three independent experiments.

FIGS. 10A-10C illustrate supporting data for the disruption of single and multiple genes in human monocyte-derived macrophages. (FIG. 10A) Histograms depicting B2M, CD14 and CD81 knockout in monocyte-derived macrophages as measured by flow cytometry. (FIG. 10B) Quantification of gene deletion. Data are mean +/- S.D. (n=3) and representative of 3 independent donors. (FIG. 10C) Assessment of gene editing efficiency by Sanger sequencing 7 days after nucleofection. Data are mean +/- S.E.M. (n=3).

FIGS. 11A-11H illustrate supporting data for the analysis of activation markers, cytokine release and phagocytosis in human monocyte-derived macrophages following RNP nucleofection. (FIG. 11A) Cell surface levels of indicated phenotypic markers measured by flow cytometry 5 days after nucleofection. (FIG. 11B) Efficiency of B2M-KO following nucleofection with two unique sgRNA:Cas9 RNPs. (FIG. 11C) Comparison of cell surface levels of CD80 and CD86 measured by flow cytometry 5 days after nucelofection. (FIG. 11D) ELISA measurements of TNF levels in cell culture media of monocyte-derived macrophages following nucleofection. (FIG. 11E) Quantification of live monocyte-derived macrophages using imaging of live cell nuclei. Micrographs depict representative images of cultured cells. (FIGS. 11F, 11G) Quantification of kinetics of particulate phagocytosis (FIG. 11F, myelin-pHrodo; FIG. 11G, beads-pHrodo). Micrographs depict representative images of phagocytosis following nucleofection, taken at the 5 hour time point. Graphs in FIGS. 11A-11C depict mean fluorescence intensity. Graphs in FIGS. 11F-11G depict intensity of pHrodo signal measured hourly following incubation with depicted particulate cargo. (FIG. 11H) Quantification of phagocytic index measured as area under curve of data in (FIG. 11F; top three bars) and (FIG. 11G; bottom three bars) over 5 hours of imaging. Data in FIGS. 11C-11H are mean +/- S.D. (n=3).

FIGS. 12A-12F illustrate the analysis of Tlr7 editing efficiency and impact of nucleofection on BMDC phenotypes. (FIG. 12A) Representative histograms of TLR7 flow cytometry following nucleofection with indicated RNP complexes using Buffer P3, Program CM-137. Quantification of TLR7-KO shown in FIG. 3A. (FIG. 12B) Frequencies of indicated myeloid cell subsets 12 days following nucleofection and Flt3 ligand-mediated BMDC differentiation. (FIG. 12C) Relative abundance of BMDCs cultured in (FIG. 12B). (FIG. 12D) Assessment of cell surface levels of indicated phenotypic and activation markers on BMDCs cultured as in (FIG. 12B). (FIGS. 12E, 12F) Quantification of CD8+ T cell/OT-I (FIG. 12E) or CD4+ T cell/OT-II (FIG. 12F) proliferation following 3 days of co-culture with BMDCs nucleofected and pulsed with indicated concentrations of ovalbumin (OVA). Histograms depict proliferation measured by CFSE dilution of OT-I or OT-II cells. Data in FIGS. 12B-12F are mean +/- S.D (n=3) and representative of two independent experiments.

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader’s convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The use of a singular indefinite or definite article (e.g., “a,” “an,” “the,” etc.) in this disclosure and in the following claims follows the traditional approach in patents of meaning “at least one” unless in a particular instance it is clear from context that the term is intended in that particular instance to mean specifically one and only one. Likewise, the term “comprising” is open ended, not excluding additional items, features, components, etc. References identified herein are expressly incorporated herein by reference in their entireties unless otherwise indicated.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by ( + ) or ( - ) 10%, 5%,1%, or any subrange or subvalue there between. Preferably, the term “about” means that the value may vary by +/- 10%.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

As used herein, the term “control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In embodiments, a control is the measurement of the expression of a gene in the absence of a compound as described herein (including embodiments and examples) In embodiments, a control is the measurement of the activity of a protein in the absence of a compound as described herein (including embodiments and examples).

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, sgRNA, guide RNA, tracrRNA, a recombinant polynucleotide, a branched polynucleotide, a plasmid, a vector, isolated DNA of a sequence, isolated RNA of a sequence, a PCR product, a nucleic acid probe, and a primer. Polynucleotides useful in the methods of the disclosure may comprise natural nucleic acid sequences and variants thereof, artificial nucleic acid sequences, or a combination of such sequences.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides, contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

The terms also encompass nucleic acids including known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine.; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids including one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “complement,” refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence.

As described herein the complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

As used herein, the term “gene” is used in accordance with its plain ordinary meaning and refers to the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following eight groups each include amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (e.g., www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

For specific proteins described herein, the named protein includes any of the protein’s naturally occurring forms, natural or engineered variants or homologs that maintain the protein’s activity (e.g., within at least 50%, 80%, 90%, 95%, 96%, 97%, 98%, 99% or 100% activity compared to the native protein). In embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form. Protein activity may be, for example, enzymatic activity or editing activity.

As used herein, the term “virus” or “virus particle” is used according to its plain ordinary meaning within the context of viral transduction. Transduction with viral vectors can be used to insert or modify genes in mammalian cells. It is often used as a tool in basic research and is actively researched as a potential means for gene therapy. However, viral transduction may have no or low efficiency or variable efficiency.

As used herein, the terms “genetic modification”, “gene modification”, “gene editing”, “genetic editing”, “genome editing”, “genome engineering” or the like refer to a type of genetic engineering in which DNA is inserted, deleted, modified or replaced at one or more specified locations in the genome of a cell. Unlike early genetic engineering techniques that randomly insert genetic material into a host genome, genome editing targets the insertions to site specific locations. One key step in gene editing is creating a double stranded break at a specific point within a gene or genome. Examples of gene editing tools such as nucleases that accomplish this step include but are not limited to Zinc finger nucleases (ZFNs), transcription activator like effector nucleases (TALEN), meganucleases, and clustered regularly interspaced short palindromic repeats system (CRISPR/Cas).

As used herein, the term “gene knockout” or “KO” refers to a genetic technique in which one of an organism’s genes is made totally or partially inoperative. A knockout can be heterozygous and homozygous KOs. In the former, only one of two gene copies (alleles) is knocked out, in the latter both are knocked out. In embodiments, near complete loss of target gene expression at the population level may be accomplished, which mitigates the need for selection steps.

The term “loss of function” as used herein refers to a mutation within a gene or deletion of a portion or the entirety or the gene that results in loss of function of the gene product or protein encoded by the gene. In embodiments, loss of function refers to decreasing or inhibiting the activity of the gene product or protein by 90%, 80%, 70%, 60%, 50%, 40%, 30%, 20%, or 10% compared to the activity of the gene product or protein in the absence of the mutation or gene deletion. In embodiments, loss of function is decreasing or inhibiting the activity of the gene product or gene by 1.5-fold, 2-fold, 3-fold, 4-fold, 5-fold, 10-fold, or more in comparison to the activity of the gene product or gene in the absence of the mutation or gene deletion.

As used herein, the term “gene editing reagent” refers to components required for gene editing tools and may include enzymes, riboproteins, solutions, co-factors and the like. For example, gene editing reagents include one or more components required for Zinc finger nucleases (ZFNs), transcription activator like effector nucleases (TALEN), meganucleases, and clustered regularly interspaced short palindromic repeats system (CRISPR/Cas) gene editing.

As used herein, the term “CRISPR” or “clustered regularly interspaced short palindromic repeats” is used in accordance with its plain ordinary meaning and refers to a genetic element that bacteria use as a type of acquired immunity to protect against viruses. CRISPR includes short sequences that originate from viral genomes and have been incorporated into the bacterial genome. Cas (CRISPR associated proteins) process these sequences and cut matching viral DNA sequences. Thus, CRISPR sequences function as a guide for Cas to recognize and cleave DNA that are at least partially complementary to the CRISPR sequence. By introducing plasmids including Cas genes and specifically constructed CRISPRs into eukaryotic cells, the eukaryotic genome can be cut at any desired position.

As used herein, the term “Cas9” or “CRISPR-associated protein 9” is used in accordance with its plain ordinary meaning and refers to an enzyme that uses CRISPR sequences as a guide to recognize and cleave specific strands of DNA that are at least partically complementary to the CRISPR sequence. Cas9 enzymes together with CRISPR sequences form the basis of a technology known as CRISPR-Cas9 that can be used to edit genes within organisms. This editing process has a wide variety of applications including basic biological research, development of biotechnology products, and treatment of diseases.

A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In aspects, the Cas9 protein is substantially identical to the protein identified by the UniProt reference number Q99ZW2 or a variant or homolog having substantial identity thereto. In aspects, the Cas9 protein has at least 75% sequence identity to the amino acid sequence of the protein identified by the UniProt reference number Q99ZW2. In aspects, the Cas9 protein has at least 80% sequence identity to the amino acid sequence of the protein identified by the UniProt reference number Q99ZW2. In aspects, the Cas9 protein has at least 85% sequence identity to the amino acid sequence of the protein identified by the UniProt reference number Q99ZW2. In aspects, the Cas9 protein has at least 90% sequence identity to the amino acid sequence of the protein identified by the UniProt reference number Q99ZW2. In aspects, the Cas9 protein has at least 95% sequence identity to the amino acid sequence of the protein identified by the UniProt reference number Q99ZW2.

A “CRISPR-associated endonuclease Cas12a,” “Cas12a,” “Cas12” or “Cas12 protein” as referred to herein includes any of the recombinant or naturally-occurring forms of the Cas12 endonuclease or variants or homologs thereof that maintain Cas12 endonuclease enzyme activity (e.g. within at least 50%, 80%, 90%, 95%, 96%, 97%, 98%, 99% or 100% activity compared to Cas12). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas12 protein. In aspects, the Cas12 protein is substantially identical to the protein identified by the UniProt reference number A0Q7Q2 or a variant or homolog having substantial identity thereto.

A “CRISPR-associated endoribonuclease Cas13a,” “Cas13a,” “Cas13” or “Cas13 protein” as referred to herein includes any of the recombinant or naturally-occurring forms of the Cas13 endoribonuclease or variants or homologs thereof that maintain Cas13 endoribonuclease enzyme activity (e.g. within at least 50%, 80%, 90%, 95%, 96%, 97%, 98%, 99% or 100% activity compared to Cas13). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas13 protein. In aspects, the Cas13 protein is substantially identical to the protein identified by the UniProt reference number P0DPB8 or a variant or homolog having substantial identity thereto.

As used herein, “Cascade” refers to the complex of Cas proteins associated with an RNA sequence including the CRISPR sequence. For example, Cascade may include one or more Cas proteins (e.g. Cas9), and cleave target DNA as directed by the CRISPR sequence. In other examples, the Cascade complex may display the CRISPR RNA and recruit Cas proteins (e.g. Cas3) to cleave the target DNA.

An “argonaut endonuclease,” “argonaut,” “protein argonaute-2” or “argonaut protein” as referred to herein includes any of the recombinant or naturally-occurring forms of the argonaut endonuclease or variants or homologs thereof that maintain argonaut endonuclease enzyme activity (e.g. within at least 50%, 80%, 90%, 95%, 96%, 97%, 98%, 99% or 100% activity compared to argonaut). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring argonaut protein. In aspects, the argonaut protein is substantially identical to the protein identified by the UniProt reference number Q9UKV8 or a variant or homolog having substantial identity thereto.

As used herein, “TALEN” or “transcription activator-like effector nuclease” refers to restriction enzymes generated by attaching a DNA binding domain (e.g. a TAL effector DNA-binding domain) to a nuclease (e.g. FokI). TALEN typically includes a naturally occurring DNA-binding domain, which include multiple modules, termed TALs or TALEs. Thus, the TALs, which include variable diresidues, confer DNA binding specificity.

As used herein, the term “donor DNA” refers to a single-stranded or double-stranded DNA that can be inserted into the genome of a cell (e.g. a myeloid cell) using genetic modification methods (e.g. CRISPR). For example, the donor DNA may have homology arms that are homolous to a region of a gene where the donor DNA is to be inserted. For example, the donor DNA may form a complex with a Cas protein. In instances, the cell may be transfected with gene editing reagents and the donor DNA.

As used herein, the term “crRNA” refers to CRISPR RNAs which are short guide RNAs including unique single repeat-spacer units. In bacterial cells, crRNA interfere with invading cognate foreign genomes by targeting the foreign DNA. Thus, short mature crRNAs are key elements in the interference step of the immune pathway. crRNA includes a nucleotide sequence at least partially complementary to the target DNA. Thus, crRNA directs target sequence recognition and enables specificity to the CRISPR gene editing mechanism. In embodiments, the crRNA may be provided as a pre-crRNA. The pre-crRNA may form a complex with an at least partially complementary region of a tracrRNA, thereby forming an RNA duplex. The pre-crRNA may be cleaved by a ribonuclease (e.g. RNase III), thus resulting in a crRNA/tracrRNA hybrid. This hybrid acts as a guide for the endonuclease Cas9, which cleaves the invading nucleic acid.

As used herein, the term “tracrRNA” or “trans-activating crRNA” refers to a small trans-encoded RNA. TracrRNA is at least partially complementary to and base pairs with a crRNA, thus forming an RNA duplex. In embodiments, the tracrRNA forms an RNA duplex with a pre-crRNA. TracrRNA may associate non-covalently with Cas (e.g. Cas9), thereby functioning as a binding scaffold for Cas. In embodiments, recognition and binding of tracrRNA by Cas results in formation of a Cas9/tracrRNA/crRNA complex.

A “guide RNA” or “gRNA” as provided herein refers to an RNA sequence having sufficient complementarity with a target polynucleotide sequence to hybridize with the target sequence and direct sequence-specific binding of a CRISPR complex to the target sequence. For example, a gRNA can direct Cas to the target polynucleotide. In embodiments, the gRNA includes the crRNA and the tracrRNA. For example, the gRNA can include the crRNA and tracrRNA hybridized by base pairing. Thus, in embodiments, the two RNA can be encoded separately by a crRNA and tracrRNA as 2 RNA molecules which then form an RNA/RNA complex due to complementary base pairing between the crRNA and tracrRNA. In aspects, the degree of complementarity between a guide RNA sequence and its corresponding target sequence, when optimally aligned using a suitable alignment algorithm, is about 50%, 60%, 75%, 80%, 85%, 90%, 95%, 97.5%, 99%, or more. In aspects, the degree of complementarity between a guide RNA sequence and its corresponding target sequence, when optimally aligned using a suitable alignment algorithm, is at least about 50%, 60%, 75%, 80%, 85%, 90%, 95%, 97.5%, 99%.

The terms “sgRNA,” “single guide RNA,” and “single guide RNA sequence” are used interchangeably and refer to an RNA sequence including the crRNA sequence and the tracrRNA sequence. For example, the sgRNA can be a single RNA sequence including the crRNA and tracrRNA. For example, the sgRNA can be a fusion sequence including the crRNA and tracrRNA. In embodiments, the sgRNA is synthesized in vitro. In embodiments, the sgRNA is made in vivo from a DNA sequence encoding the sgRNA.

In embodiments, the methods provided herein are used in combination with a Type II CRISPR system to generate single and/or double strand breaks in the host genome. In particular embodiments, a nuclease, such as the Cas9 nuclease, is guided to a target site by a guide RNA (e.g. crRNA hybridized to tracrRNA). The guide RNA and the nuclease form a co-localization complex at the DNA, upon which the nuclease induces breaks in the target DNA. In the example embodiments, where the nuclease is Cas9, the Cas9 generates a blunt-ended double-stranded break 3 bp upstream of a protospacer-adjacent motif (PAM) in the target genome via a process mediated by two catalytic domains in the protein.

Non-limiting examples of CRISPR enzymes include Cas1, Cas1B, Cas2, Cas3, Cas4, Cas5, Cas6, Cas7, Cas8, Cas9 (also known as Csn1 and Csx12), Cas10, Cas12, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In embodiments, the CRISPR enzyme is a Cas9 enzyme. In embodiments, the Cas9 enzyme is S. pneumoniae, S. pyogenes or S. thermophilus Cas9, or mutants derived thereof in these organisms. In embodiments, the CRISPR enzyme is codon-optimized for expression in a eukaryotic cell. In embodiments, the CRISPR enzyme directs cleavage of one or two strands at the location of the target sequence. In embodiments, the CRISPR enzyme lacks DNA strand cleavage activity.

As used herein, a “zinc finger” is a polypeptide structural motif folded around a bound zinc cation. In embodiments, the polypeptide of a zinc finger has a sequence of the form X₃-Cys-X₂₋₄ -Cys-X₁₂-His-X₃₋₅-His-X₄, wherein X is any amino acid (e.g., X₂₋₄ indicates an oligopeptide 2-4 amino acids in length). Thus, “zinc finger nuclease” as used herein refers to a nuclease including a zinc finger motif and a domain capable of inducing breaks in the target DNA.

Non-limiting examples of methods for homologous recombination and gene editing using various nuclease systems can be found, for example, in U.S. Pat. No. 8945839, International PCT application Pub. No. WO2013/163394 and U.S. Pat. Application Nos. 2016/0060657, 2012/0192298A1 and US2007/0042462, each of which is herein incorporated by reference in its entirety. These and any other known methods for homologous recombination can be used with the plasmid vectors provided herein.

As used herein, the term “myeloid cell” is used in accordance with its plain ordinary meaning and refers to any cell derived from and including myeloid stem cells. In embodiments, myeloid stem cells are derived from hematopoietic stem cells. Myeloid cells are progenitor cells of different types of cells. They produce many different types of blood cells including monocytes, macrophages, neutrophils, basophils, eosinophils, erythrocytes, dendritic cells, megakaryocytes, and platelets.

As used herein, the term “monocyte” or “monocyte cell” is used in accordance with its plain ordinary meaning and refers to a type of leukocyte, or white blood cell. They are the largest type of leukocyte and can differentiate into macrophages and myeloid lineage dendritic cells. As a part of the vertebrate innate immune system monocytes also influence the process of adaptive immunity. There are at least three types of monocytes in human blood: 1) The classical monocyte may be characterized by high level expression of the CD14 cell surface receptor (CD14⁺⁻ CD16⁻ monocyte); 2) The non-classical monocyte shows low level expression of CD14 and additional co-expression of the CD16 receptor (CD14⁺CD16⁺⁺ monocyte); and 3) The intermediate monocyte with high level expression of CD14 and low level expression of CD16 (CD14⁺⁺CD16⁺ monocytes).

As used herein, the term “macrophage” is used in accordance with its plain ordinary meaning and refers to a type of white blood cell of the immune system, that in a process called phagocytosis, engulfs and digests cellular debris, foreign substances, microbes, cancer cells, and anything else that does not have the type of proteins specific to healthy body cells on its surface. Beyond increasing inflammation and stimulating the immune system, macrophages also play an important anti-inflammatory role and can decrease immune reactions through the release of cytokines.

As used herein, the term “dendritic cell” is used in accordance with its plain ordinary meaning and refers to are antigen-presenting cells (also known as accessory cells) of the mammalian immune system. Their main function is to process antigen material and present it on the cell surface to the T cells of the immune system. They act as messengers between the innate and the adaptive immune systems. The most common division of dendritic cells is “myeloid” vs. “plasmacytoid dendritic cell” (lymphoid). In embodiments, dendritic cells herein are myeloid dendritic cells.

As used herein, the term “electroporation”, “electropermeabilization”, and “electrotransfer” are used in accordance with its plain ordinary meaning and refer to a technique in which an electrical field is applied to cells in order to increase the permeability of the cell membrane, allowing chemicals, drugs, proteins, or nucleic acids, or combinations thereof to be introduced into the cell. Afterwards, the cells have to be handled carefully until they have had a chance to divide. This process is approximately ten times more effective than chemical transformation. Thus, the term “electroporation enhancer” refers to a compound or composition that improves the delivery of of a chemical, drug compound, protein or nucleic acid into a cell, improves efficiency of genetic modification of a cell and/or increases the level of cell viability following transfection. In embodiments, the electroporation enhancer improves the delivery of of a chemical, drug compound, protein or nucleic acid into a cell. In embodiments, the electroporation enhancer increases the efficiency of genetic modification in a cell. In embodiments, the electroporation enhancer increases the efficiency of genetic modification in a cell compared to the efficiency of genetic modification in the absence of the electroporation enhancer. In embodiments, the electroporation enhancer increases the level of cell viablitiy following transfection. In embodiments, the electroporation enhancer increases the level of cell viablitiy following transfection cell compared to cell viability after transfection in the absence of the electroporation enhancer.

As used herein, the term “transfection” is used in accordance with its plain ordinary meaning and refers to a process of deliberately introducing naked or purified nucleic acids into eukaryotic cells. In instances, “transfection” may refer to other methods and cell types, although other terms are often preferred. For example, the term “transformation” is typically used to describe non-viral DNA transfer in bacteria and non-animal eukaryotic cells, including plant cells. In animal cells, transfection is the preferred term. For example, the term “transduction” is often used to describe virus-mediated gene transfer into eukaryotic cells.

A “transfection reagent” can be any compound and/or composition that increases the uptake of one or more nucleic acids into one or more target cells.

As used herein, the term “contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “modulate” is used in accordance with its plain ordinary meaning and refers to the act of changing or varying one or more properties. “Modulation” refers to the process of changing or varying one or more properties. For example, as applied to the effects of a modulator on a target gene, to modulate means to change by increasing or decreasing expression of the gene or the activity of the gene.

The term “aberrant” as used herein refers to different from normal. When used to describe enzymatic activity, aberrant refers to activity that is greater or less than a normal control or the average of normal non-diseased control samples. Aberrant activity may refer to an amount of activity that results in a disease, wherein returning the aberrant activity to a normal or non-disease-associated amount (e.g. by using a method as described herein), results in reduction of the disease or one or more disease symptoms.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid including two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein including two or more subsequences that are not found in the same relationship to each other in nature (e.g., a fusion protein).

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

As used herein, the term “administering” means a suitable route for cellular therapy. Examples include intravenous (IV), intramuscular (IM), intrathecal (lumbar puncture), or interarterial (IA) administration. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, etc. In embodiments, the administering does not include administration of any active agent other than the recited active agent. In embodiments, the administering includes co-administration with another agent. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Co-administration is meant to include simultaneous or sequential administration of the compounds individually or in combination (more than one compound). Thus, the preparations can also be combined, when desired, with other active substances (e.g. to reduce metabolic degradation).

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer’s, normal sucrose, normal glucose, binders, fillers, disintegrants, lubricants, coatings, sweeteners, flavors, salt solutions (such as Ringer’s solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Leukemias that may be treated with a compound or method provided herein include, for example, acute myeloid leukemia, chronic myeloid leukemia, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross’ leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling’s leukemia, stem cell leukemia, subleukemic leukemia, or undifferentiated cell leukemia.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Methods for Genetic Modification

Provided herein are methods for genetically modifying myeloid cells without using viral transduction methods for delivering gene editing reagents into the cells. Applicant has discovered methods which have surprisingly overcome issues, including inefficient or variably efficient delivery of compounds (e.g. oligonucleotides, proteins, etc.), associated with prior delivery methods. The methods provided herein including embodiments thereof consistently modify myeloid cells to near-population level. Therefore, the cells may not need enrichment or selection steps following delivery of the gene editing reagents into the cells Thus, in an aspect, provided herein are methods for genetic modification of a myeloid cell including transfecting the myeloid cell with a gene editing reagent targeting a site of interest, where the myeloid cell is not transduced with a viral vector.

In embodiments of the methods of genetic modification provided herein, the myeloid cell is a primary myeloid cell. In embodiments of the methods of genetic modification provided herein, the myeloid cell is a passaged myeloid cell.

In embodiments of the methods of genetic modification provided herein, the myeloid cell is a a monocyte, macrophage, neutrophil, basophil, eosinophil, erythrocyte, dendritic cell, or megakaryocyte. In embodiments, the myeloid cell is a monocyte. In embodiments, the myeloid cell is a macrophage. In embodiments, the myeloid cell is a dendritic cell. In embodiments, the myeoid cell is a neutrophil. In embodiments, the myeoid cell is a basophil. In embodiments, the myeoid cell is an eosinophil. In embodiments, the myeoid cell is an erythrocyte. In embodiments, the myeoid cell is a megakaryocyte. In embodiments, the myeloid cell is a promyelocyte, promonocyte, myeloid stem cell, proerythroblast, or promegakaryoyte. In embodiments, the myeloid cell is a promyelocyte. In embodiments, the myeloid cell is a promonocyte. In embodiments, the myeloid cell is a myeloid stem cell. In embodiments, the myeloid cell is a proerythroblast. In embodiments, the myeloid cell is a promegakaryoyte.

In embodiments of the methods of genetic modification provided herein, the cell is transfected via electroporation using an electroporation system. In embodiments of the methods of genetic modification provided herein, the cell is transfected via nucleofection using a nucleofection system. As used herein, “nucleofection” referes to an electroporation-based transfection method used to delivery nucleic acids (e.g. DNA) into cells. For example, nucleofection allows delivery of of DNA into the cytoplasm or nucleus of a myeloid cell. In embodiments, the nucleofection system is one of several Nucleofector™ systems provided by Lonza (Basel, Switzerland). In embodiments, the cell is transfected via nucleofection.

In embodiments, the transfection step is accomplished using a chemical transfection system, such as, e.g., a polymer-based transfection reagents. A variety of polymer-cased transfection reagents are known to those skilled in the art. In embodiments, the transfection reagent is a polymer-based transfection reagent. Suitable transfection reagents can include, but are not limited to, one or more compounds and/or compositions comprising cationic polymers such as polyethyleneimine (PEI), polymers of positively charged amino acids such as polylysine and polyarginine, positively charged dendrimers and fractured dendrimers, cationic B-cyclodextrin including polymers (CD-polymers), DEAE-dextran, TURBOFECT™ transfection reagents (available from ThermoFisher Scientific), Xfect transfection reagent (available from Takara Bio USA), Sigma Universal Transfection Reagent (available from Sigma-Aldrich). In embodiments, the transfection reagent is a cationic polymer-based transfection reagent, for example, polyglycolic acid (PGA), POLYMER In Vivo Transfection Reagent (available from Altogen Biosystems); and polyethylenimine (PEI), and the like. In embodiments, the transfection reagent is a cationic polymer-based transfection reagent, for example, polyglycolic acid (PGA), POLYMER In Vivo Transfection Reagent (available from Altogen Biosystems); and polyethylenimine (PEI).

In embodiments of the methods of genetic modification provided herein, the cell is transfected using a lipid-based transfection system. In embodiments, a reagent for the introduction of macromolecules into cells can comprise one or more lipids which can be cationic lipids and/or neutral lipids. Example lipids include, but are not limited to, N-[1-(2,3-dioleyloxy) propyl]-N,N,N-trimethylamonium chloride (DOTMA), dioleoylphosphatidylcholine (DOPE), 1,2-Bis(oleoyloxy)-3-(4′-trimethylammonio) propane (DOTAP), dihydroxyl-dimyristylspermine tetrahydrochloride (DHDMS), hydroxyl-dimyristylspermine tetrahydrochloride (HDMS), 1,2-dioleoyl-3-(4′-trimethylammonio) butanoyl-sn-glycerol (DOTB), 1,2-dioleoyl-3-succinyl-sn-glycerol choline ester (DOSC), cholesteryl (4′-trimethylammonio)butanoate (ChoTB), cetyltrimethylammonium bromide (CTAB), 1,2-dioleoyl-3-dimethyl-hydroxyethyl ammonium bromide (DORI), 1,2-dioleyloxypropyl-3 -dimethyl-hydroxyethyl ammonium bromide (DOME), 1,2-dimyristyloxypropyl-3-dimethylhydroxyethyl ammonium bromide (DMRIE), O,O′-didodecyl-N-[p(2-trimethylammonioethyloxy)benzoyl]-N,N,N-trimethylammonium chloride, spermine conjugated to one or more lipids (for example, 5-carboxyspermylglycine dioctadecylamide (DOGS), N,N^{I},N^{II},N^{III}-tetramethyl-N,N^{I},N^{II},N^{III}-tet-rapalmitylspermine (TM-TPS) and dipalmitoylphasphatidylethanolamine 5-carboxyspermylaminde (DPPES)), lipopolylysine (polylysine conjugated to DOPE), TRIS (Tris(hydroxymethyl)aminomethane, tromethamine) conjugated fatty acids (TFAs) and/or peptides such as trilysyl-alanyl-TRIS mono-, di-, and tri-palmitate, (3B-[N-(N′,N′-dimethylaminoethane)-carbamoyl] cholesterol (DCChol), N-(α-trimethylammonioacetyl)-didodecyl-D-glutamate chloride (TMAG), dimethyl dioctadecylammonium bromide (DDAB), 2,3-dioleyloxy-N- [2(spermine-carboxamido)ethyl] -N,N-dimethyl- 1-propanamin-iniumtrinuoroacetate (DOSPA) and combinations thereof. Those skilled in the art will appreciate that certain combinations of the above-mentioned lipids for example, cationic lipids, have been shown to be particularly suited for the introduction of nucleic acids, proteins, ribonucleic proteins, etc. into cells. In embodiments, the transfection reagent is a cationic lipid transfection reagent. Examples of cationic lipid transfection reagent suited for introduction of nucleic acids into cells inlude a 3:1 (w/w) combination of DOSPA and DOPE available from Life Technologies Corporation, Carlsbad, Calif. under the trade name LIPOFECTAMINE™, a 1:1 (w/w) combination of DOTMA and DOPE available from Life Technologies under the trade name LIPOFECTIN®, a 1:1 (M/M) combination of DIVIRIE and cholesterol available from Life Technologies Corporation, Carlsbad, Calif. under the trade name DIVIRIE-C reagent; a 1:1.5 (M/M) combination of TM-TPS and DOPE available from Life Technologies, and CRISPRMAX™ available from Life Technologies Corporation, Carlsbad, Calif. Other commercially available cationic lipid transfection reagents include, without limitation, TRANSFAST™ (available from Promega Corporation); LYOVEC™ (available from InvivoGen); DOTAP liposomal transfection reagent (available from Roche); TRANSIT® transfection reagents (available from Mirus); and GENEJUICE® Transfection Reagent (EMD Millipore). Additional transfection reagents that may be used herein include, without limitation, VIAFECT™ Transfection Reagent, FUGENE® 6 Transfection Reagent, and FUGENE® HD Transfection Reagent, each of which is available from Promega Corporation; and TRANSFECTIN™ Lipid Reagent, available from BioRad Laboratories, Inc.

In embodiments of the methods of genetic modification provided herein, the cell is not subjected to a selection step and/or an enrichment step after genetic modification. In embodiments, the cell is not subjected to a selection step after genetic modification. A selection step may be a positive or negative selection for a phenotype of interest. For example, selection may be based on antibiotic resistance. In embodiments, the cell is not subjected to an enrichment step after transfection. In embodiments, an enrichments step is a process that enriches or expands a population of cells of interest or cells obtained from a selection step. In embodiments of the methods of genetic modification provided herein, the cell is not subjected to both of a selection step and an enrichment step after transfection, thus significantly improving efficiency and decreasing time required for obtaining the genetically modified cells.

In embodiments, the methods described herein include genetic modification methods. In embodiments, genetic modification methods provided herein include gene editing. In embodiments, gene editing methods provided herein include nucleases. In embodiments, methods for genetic modification provided herein include nucleases for gene editing, for example, zinc finger nucleases (ZFNs), transcription activator like effector nucleases (TALEN), meganucleases, and clustered regularly interspaced short palindromic repeats system (CRISPR/CASR), and variants thereof readily identified by those skilled in the art. In embodiments, genetic modification methods provided herein include the CRISPR-CAS system.

In embodiments genetic modification methods provided herein include a gene editing reagent. In embodiments, the gene editing reagent includes an RNA-guided nuclease. In embodiments, the RNA-guided nuclease is a CRISPR-Cas system. In embodiments, the CRISPR-Cas system includes a Cas9 or a Cas9 variant. In embodiments, the CRISPR-Cas system includes a Cas9. In embodiments, the CRISPR-Cas system includes a Cas9 variant. In embodiments, the CRISPR-Cas system includes a Cas12, Cascade, a Cas13, or a variant of each thereof. In embodiments, the CRISPR-Cas system includes a Cas12. In embodiments, the CRISPR-Cas system includes Cascade. In embodiments, the CRISPR-Cas system includes a Cas13. In embodiments, the CRISPR-Cas system includes a Cas12 variant. In embodiments, the CRISPR-Cas system includes Cascade, wherein one or more member of the Cascade complex is a variant. In embodiments, the CRISPR-Cas system includes a a Cas13 variant.

In embodiments of the methods of genetic modification provided herein, the gene editing reagent includes a CRISPR-Cas system including a Cas protein, a guide RNA, and optionally a donor DNA. In embodiments, the Cas protein and guide RNA are non-covalently associated. In embodiments, the Cas protein, guide RNA and donor DNA are non-covalently associated.

In embodiments, the gene editing reagent includes transcription activator-like effector nuclease (TALEN), a zinc finger nuclease, or an Argonaut endonuclease. In embodiments, the gene editing reagent includes transcription activator-like effector nuclease (TALEN). In embodiments, the gene editing reagent includes a zinc finger nuclease. In embodiments, the gene editing reagent includes an Argonaut endonuclease.

In embodiments of the methods of genetic modification provided herein which employ electroporation/nucleofection, the cells may be contacted with an electroporation enhancer during transfection. In embodiments, the electroporation enhancer is a carrier DNA, a single stranded DNA, a combination of single stranded and double stranded DNA, a polymeric additive, and/or an oligonucleotide. In embodiments, the electroporation enhancer is a single stranded DNA. In embodiments, the electroporation enhancer is a combination of single stranded and double stranded DNA. In embodiments, the electroporation enhancer is polymeric additive. In embodiments, the electroporation enhancer is an oligonucleotide. In embodiments, the oligonucleotide is a TLR antagonist, such as A151. In embodiments, the carrier DNA is a single-stranded DNA oligonucleotide. In embodiments, the carrier DNA is a double-stranded DNA oligonucleotide. In embodiments, the carrier DNA is non-homologous to a human, mouse, and/or rat genomes. In embodiments, the carrier DNA is non-homologous to a human genome. In embodiments, the carrier DNA is non-homologous to a mouse genome. In embodiments, the carrier DNA is non-homologous to a rat genome. In embodiments, electroporation is enhanced by the use of one or more JAK2 inhibitors, e.g. when Cas mRNA is delivered into the cell.

In embodiments of the methods of genetic modification provided herein, the myeloid cell is differentiated prior to electroporation. In embodiments, the myeloid cell is differentiated into a dendritic cell. In embodiments, the myeloid cell is differentiated into a macrophage. In embodiments, freshly isolated monocytes (human) are electroporated, then differentiated into macrophages. Methods for differentiating myeloid cells are well known in the art. See, e.g., Harada, Y., et al. Cytokine-based high log-scale expansion of functional human dendritic cells from cord-blood CD34-positive cells. Sci Rep 1, 174 (2011); Ohradanova-Repic A, et al. Differentiation of human monocytes and derived subsets of macrophages and dendritic cells by the HLDA10 monoclonal antibody panel. Clin Transl Immunology. 2016;5(1):e55; each of which is incorporated by reference in its entirety.

In embodiments of the methods of genetic modification provided herein, the myeloid cell is not differentiated prior to electroporation. In embodiments, total bone marrow cells (for example, mouse) are electroporated, then differentiated into bone marrow derived dentritic cells (BMDCs).

Myeloid cells may be activated by exposure of the cells to various factors, including viruses. However, activation of myeloid cells, e.g. during genetic engineering of the cells, reduces their usefulness in downstream applications. In embodiments of the methods of genetic modification provided herein, the myeloid cell is not activated prior to or during genetic modification. In embodiments of the methods of genetic modification provided herein, the myeloid cell is not activated prior to genetic modification. In embodiments of the methods of genetic modification provided herein, the myeloid cell is modestly activated during genetic modification. In embodiments, modestly may include slight activation or activation that is insignificant. For example, absence or decreased expression of certain markers may indicate that a myeloid cell or plurality of myeloid cells are not activated, are slightly activated or are insignificantly activated. For example, CD64, CD169 or HLA-DR expression may be decreased or absent in a myeloid cell or within a plurality of myeloid cells.

In embodiments of the methods of genetic modification provided herein, two or more distinct crRNAs complementary to the site of interest are introduced into the myeloid cell. In embodiments of the methods of genetic modification provided herein, two distinct crRNAs complementary to the site of interest are introduced into the myeloid cell. In embodiments of the methods of genetic modification provided herein, three distinct crRNAs complementary to the site of interest are introduced into the myeloid cell. In embodiments of the methods of genetic modification provided herein, four distinct crRNAs complementary to the site of interest are introduced into the myeloid cell. In embodiments of the methods of genetic modification provided herein, five distinct crRNAs complementary to the site of interest are introduced into the myeloid cell. In embodiments of the methods of genetic modification provided herein, six distinct crRNAs complementary to the site of interest are introduced into the myeloid cell. In embodiments of the methods of genetic modification provided herein, seven distinct crRNAs complementary to the site of interest are introduced into the myeloid cell. In embodiments of the methods of genetic modification provided herein, eight distinct crRNAs complementary to the site of interest are introduced into the myeloid cell.

In embodiments, introducing the crRNAs into the myeloid cell includes transfecting the cell with the crRNAs. In embodiments, the crRNAs are contacted with tracrRNAs. In embodiments, contacting the crRNAs includes annealing crRNAs to the tracrRNAs. In embodiments, the crRNAs are single guide RNAs (sgRNAs).

In embodiments of the methods of genetic modification provided herein, multiple sites of interest are targeted. In embodiments of the methods of genetic modification provided herein, two sites of interest are targeted. In embodiments of the methods of genetic modification provided herein, three sites of interest are targeted. In embodiments of the methods of genetic modification provided herein, four sites of interest are targeted. In embodiments of the methods of genetic modification provided herein, five sites of interest are targeted. In embodiments of methods of genetic modification where multiple sites of interest are targeted, two or more distinct crRNAs to each site of interest are introduced into the myeloid cell.

In embodiments of the methods of genetic modification provided herein, the myeloid cell is a mammalian cell. In embodiments, the mammalian myeloid cell is selected from a bovine, rat, mouse, dog, monkey, goat, sheep, cow, deer, or other mammalian myeloid cell. In embodiments, the mammalian myeloid cell is a bovine myeloid cell. In embodiments, the mammalian myeloid cell is a rat myeloid cell. In embodiments, the mammalian myeloid cell is a mouse myeloid cell. In embodiments, the mammalian myeloid cell is a dog myeloid cell. In embodiments, the mammalian myeloid cell is a monkey myeloid cell. In embodiments, the mammalian myeloid cell is a goat myeloid cell. In embodiments, the mammalian myeloid cell is a sheep myeloid cell. In embodiments, the mammalian myeloid cell is a cow myeloid cell. In embodiments, the mammalian myeloid cell is a cow myeloid cell. In embodiments, the mammalian myeloid cell is a deer myeloid cell. In embodiments, the myeloid cell is a non-mammalian myeloid cell. In embodiments, myeloid cell is a human myeloid cell.

In an aspect, provided herein are methods for genetic modification of a plurality of myeloid cells, including transfecting the plurality of myeloid cells in the presence of a gene editing reagent targeting a site of interest, where the myeloid cells are not transduced with a viral vector. In embodiments of the methods of genetic modification provided herein, the plurality of myeloid cells are cultured myeloid cells. In embodiments of the methods of genetic modification provided herein, the plurality of myeloid cells is transfected via electroporation. In embodiments of the methods of genetic modification provided herein, the plurality of myeloid cells is transfected via lipid-based transfection.

In embodiments, of the method of genetic modification provided herein, the plurality of myeloid cells are not activated prior to or during genetic modification. In embodiments, at least 50%, 55%, 60%, 65%, 70%, 75%, 80%, 85%, 90%, 91%, 92%, 93%, 94%, 95%, 96%, 97%, 98%, 99% or 100% of the myeloid cells within the plurality of myeloid cells are not activated. In embodiments, at least 80%, 85%, 90%, 95%, 96%, 97%, 98%, 99% or 100% of the myeloid cells within the plurality of myeloid cells are not activated. In embodiments, at least 90%, 95%, 96%, 97%, 98%, 99% or 100% of the myeloid cells within the plurality of myeloid cells are not activated. For example, absence or decreased expression of certain markers may indicate that a myeloid cell or plurality of myeloid cells are not activated, are slightly activated or are insignificantly activated. For example, CD64, CD169 or HLA-DR expression may be decreased or absent in a myeloid cell or within the plurality of myeloid cells.

For the methods provided herein including embodiments thereof, near population-level genetic knockout of single and multiple targets can be achieved in a range of cell types without the need for selection or enrichment of the genetically modified cells. As used herein, “near-population level” refers to substantially all cells in a given population of cells. Thus, in embodiments, near-population level refers to at least 70% of a population of myeloid cells. Thus, in embodiments, near-population level refers to at least 75% of a population of myeloid cells. Thus, in embodiments, near-population level refers to at least 80% of a population of myeloid cells. Thus, in embodiments, near-population level refers to at least 85% of a population of myeloid cells. Thus, in embodiments, near-population level refers to at least 90% of a population of myeloid cells. Thus, in embodiments, near-population level refers to at least 95% of a population of myeloid cells. In embodiments, near-population level is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of a population of myeloid cells. In embodiments, near-population level is at least 90%, 95%, 96%, 97%, 98%, 99% or 100% of a population of myeloid cells.

Thus, in embodiments of the methods of genetic modification provided herein, the site of interest is modified in at least 70% of the plurality of myeloid cells. In embodiments of the methods of genetic modification provided herein, the site of interest is modified in at least 80% of the plurality of myeloid cells. In embodiments of the methods of genetic modification provided herein, the site of interest is modified in at least 85% of the plurality of myeloid cells. In embodiments of the methods of genetic modification provided herein, the site of interest is modified in at least 90% of the plurality of myeloid cells. In embodiments of the methods of genetic modification provided herein, the site of interest is modified in at least 95% of the plurality of myeloid cells. In embodiments, the site of interest is modified in at least 71%, 72%, 73%, 74%, 75%, 76%, 77%, 78%, 79%, 80%, 81%, 82%, 83%, 84%, 85%, 86%, 87%, 88%, 89, 90%, 91%, 92%, 93%, 94%, 95%, 96%, 97%, 98%, or 99% of the plurality of myeloid cells.

In embodiments of the methods of genetic modification provided herein, the plurality of myeloid cells includes dendritic cells (DCs) and the site of interest is modified in at least 50% of the DCs. In embodiments of the methods of genetic modification provided herein, the plurality of myeloid cells includes dendritic cells (DCs) and the site of interest is modified in at least 60% of the DCs. In embodiments of the methods of genetic modification provided herein, the plurality of myeloid cells includes dendritic cells (DCs) and the site of interest is modified in at least 70% of the DCs. In embodiments of the methods of genetic modification provided herein, the plurality of myeloid cells includes dendritic cells (DCs) and the site of interest is modified in at least 80% of the DCs. In embodiments of the methods of genetic modification provided herein, the plurality of myeloid cells includes dendritic cells (DCs) and the site of interest is modified in at least 90% of the DCs. In embodiments of the methods of genetic modification provided herein, the plurality of myeloid cells includes dendritic cells (DCs) and the site of interest is modified in at least 95% of the DCs. In embodiments, the plurality of myeloid cells includes dendritic cells (DCs) and the site of interest is modified in at least 55%, 60%, 70%, 75%, 80%, 85%, 90%, or 95% of the DCs. In embodiments, the plurality of myeloid cells includes dendritic cells (DCs) and the site of interest is modified in at least 51%,52%, 53%, 54%, 55%, 56%, 57%, 58%, 59%, 60%, 61%, 62%,63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89, 90%, 91%, 92%, 93%, 94%, 95%, 96%, 97%, 98%, or 99% of the of the DCs.

The methods described herein can be used to modify a single gene or a plurality of genes simultaneously in a myeloid cell. One gene, two genes, three genes, or more than three genes can be modified simultaneously in the myloid cell. Using the methods provided herein including embodiments thereof, up to 20 genes can be modified simultaneously in a myeloid cell.

In embodiments of the methods of genetic modification provided herein, the viability of the plurality of myeloid cells after electroporation is at least 81%, 82%, 83%, 84%, 85%, 86%, 87%, 88%, 89, 90%, 91%, 92%, 93%, 94%, 95%, 96%, 97%, 98%, or 99%. In embodiments of the methods of genetic modification provided herein, the viability of the plurality of myeloid cells after electroporation is at least 80%. In embodiments of the methods of genetic modification provided herein, the viability of the plurality of myeloid cells after electroporation is at least 85%. In embodiments of the methods of genetic modification provided herein, the viability of the plurality of myeloid cells after electroporation is at least 90%. In embodiments of the methods of genetic modification provided herein, the viability of the plurality of myeloid cells after electroporation is at least 95%. Viability can be measured by any technique known to those skilled in the art and include, but is not limited to, cytolysis, caspase, functional, genomic, proteomic, and/or flow cytometry assays.

In embodiments of the methods of genetic modification provided herein, the gene editing reagent includes an RNA-guided nuclease. In embodiments, the RNA-guided nuclease is a CRISPR system. For the methods provided herein, in embodiments, the CRISPR system includes Cas1, Cas1B, Cas2, Cas3, Cas4, Cas5, Cas6, Cas7, Cas8, Cas9 (also known as Csn1 and Csx12), Cas10, Cas12, Cas13, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In embodiments, the RNA-guided nuclease is a CRISPR-Cas system. In embodiments, the CRISPR-Cas system includes a Cas9, a Cas12, a Cascade, a Cas13, or a variant of each thereof. In embodiments, the CRISPR-Cas system includes a Cas9 or a Cas9 variant. In embodiments, the CRISPR-Cas system includes a Cas12, a Cascade, a Cas13, or a variant of each thereof. In embodiments of the methods of genetic modification provided herein, the gene editing reagent includes a CRISPR-Cas system that includes Cas9. In embodiments of the methods of genetic modification provided herein, the gene editing reagent includes a CRISPR-Cas system that includes a Cas9 variant. In embodiments of the methods of genetic modification provided herein, the gene editing reagent includes a CRISPR-Cas system that includes Cas12 or variant thereof. In embodiments of the methods of genetic modification provided herein, the gene editing reagent includes a CRISPR-Cas system that includes a Cascade or variant thereof. In embodiments of the methods of genetic modification provided herein, the gene editing reagent includes Cas3. In embodiments of the methods of genetic modification provided herein, the gene editing reagent includes a CRISPR-Cas system that includes Cas13 or a variant thereof.

For the methods provided herein, in embodiments, the gene editing reagent includes a CRISPR-Cas system including a Cas protein, a guide RNA, and optionally a donor DNA. In embodiments, the Cas protein and guide RNA are non-covalently associated. In embodiments, the Cas protein, guide RNA and donor DNA are non-covalently associated.

In embodiments of the methods of genetic modification provided herein, the gene editing reagent includes a transcription activator-like effector nuclease (TALEN), a zinc finger nuclease, or an Argonaut endonuclease. In embodiments, the gene editing reagent includes a TALEN. In embodiments, the gene editing reagent includes a zinc finger nuclease. In embodiments, the gene editing reagent includes an Argonaut endonuclease.

In embodiments of the methods of genetic modification provided herein, the gene editing reagent includes an RNA-guided nuclease. In embodiments, the RNA-guided nuclease includes a guide RNA and a ribonucleoprotein (RNP), wherein the ratio of guide RNA to ribonucleoprotein (RNP) is less than or equal to about 100:1 to about 1:100. In embodiments of the methods of genetic modification provided herein, the gene editing reagent includes an RNA-guided nuclease and a ribonucleoprotein (RNP), where the ratio of guide RNA to RNP is less than or equal to about 100:1, less than or equal to about 90:1, less than or equal to about 80:1, less than or equal to about 70:1, less than or equal to about 60:1, less than or equal to about 50:1, less than or equal to about 40:1, less than or equal to about 30:1, less than or equal to about 20:1, less than or equal to about 10:1, less than or equal to about 1:10, less than or equal to about 1:20, less than or equal to about 1:30, less than or equal to about 1:40, less than or equal to about 1:50, less than or equal to about 1:60, less than or equal to about 1:70, less than or equal to about 1:80, less than or equal to about 1:90, or less than or equal to about 1:100. In embodiments of the methods of genetic modification provided herein, the gene editing reagent includes an RNA-guided nuclease and a ribonucleoprotein (RNP), where the ratio of guide RNA to RNP is less than or equal to about 100:1. In embodiments of the methods of genetic modification provided herein, the gene editing reagent includes an RNA-guided nuclease and a ribonucleoprotein (RNP), where the ratio of guide RNA to RNP less than or equal to about 90:1. In embodiments of the methods of genetic modification provided herein, the gene editing reagent includes an RNA-guided nuclease and a ribonucleoprotein (RNP), where the ratio of guide RNA to RNP less than or equal to about 80:1. In embodiments of the methods of genetic modification provided herein, the gene editing reagent includes an RNA-guided nuclease and a ribonucleoprotein (RNP), where the ratio of guide RNA to RNP less than or equal to about 70:1. In embodiments of the methods of genetic modification provided herein, the gene editing reagent includes an RNA-guided nuclease and a ribonucleoprotein (RNP), where the ratio of guide RNA to RNP less than or equal to about 60:1. In embodiments of the methods of genetic modification provided herein, the gene editing reagent includes an RNA-guided nuclease and a ribonucleoprotein (RNP), where the ratio of guide RNA to RNP less than or equal to about 50:1. In embodiments of the methods of genetic modification provided herein, the gene editing reagent includes an RNA-guided nuclease and a ribonucleoprotein (RNP), where the ratio of guide RNA to RNP less than or equal to about 40:1. In embodiments of the methods of genetic modification provided herein, the gene editing reagent includes an RNA-guided nuclease and a ribonucleoprotein (RNP), where the ratio of guide RNA to RNP less than or equal to about 30:1. In embodiments of the methods of genetic modification provided herein, the gene editing reagent includes an RNA-guided nuclease and a ribonucleoprotein (RNP), where the ratio of guide RNA to RNP less than or equal to about 20:1. In embodiments of the methods of genetic modification provided herein, the gene editing reagent includes an RNA-guided nuclease and a ribonucleoprotein (RNP), where the ratio of guide RNA to RNP less than or equal to about 10:1.

In embodiments of the methods of genetic modification provided herein, the gene editing reagent includes an RNA-guided nuclease and a ribonucleoprotein (RNP), where the ratio of guide RNA to RNP less than or equal to about 1:10. In embodiments of the methods of genetic modification provided herein, the gene editing reagent includes an RNA-guided nuclease and a ribonucleoprotein (RNP), where the ratio of guide RNA to RNP less than or equal to about 1:20. In embodiments of the methods of genetic modification provided herein, the gene editing reagent includes an RNA-guided nuclease and a ribonucleoprotein (RNP), where the ratio of guide RNA to RNP less than or equal to about 1:30. In embodiments of the methods of genetic modification provided herein, the gene editing reagent includes an RNA-guided nuclease and a ribonucleoprotein (RNP), where the ratio of guide RNA to RNP less than or equal to about 1:40. In embodiments of the methods of genetic modification provided herein, the gene editing reagent includes an RNA-guided nuclease and a ribonucleoprotein (RNP), where the ratio of guide RNA to RNP less than or equal to about 1:50. In embodiments of the methods of genetic modification provided herein, the gene editing reagent includes an RNA-guided nuclease and a ribonucleoprotein (RNP), where the ratio of guide RNA to RNP less than or equal to about 1:60. In embodiments of the methods of genetic modification provided herein, the gene editing reagent includes an RNA-guided nuclease and a ribonucleoprotein (RNP), where the ratio of guide RNA to RNP less than or equal to about 1:70. In embodiments of the methods of genetic modification provided herein, the gene editing reagent includes an RNA-guided nuclease and a ribonucleoprotein (RNP), where the ratio of guide RNA to RNP less than or equal to about 1:80, less than or equal to about 1:90. In embodiments of the methods of genetic modification provided herein, the gene editing reagent includes an RNA-guided nuclease and a ribonucleoprotein (RNP), where the ratio of guide RNA to RNP less than or equal to about 1:100. In embodiments of the methods of genetic modification provided herein, the gene editing reagent includes an RNA-guided nuclease and a ribonucleoprotein (RNP), where the ratio of guide RNA to RNP less than or equal to about 3:1. In embodiments of the methods of genetic modification provided herein, the gene editing reagent includes an RNA-guided nuclease and a ribonucleoprotein (RNP), where the ratio of guide RNA to RNP less than or equal to about 2:1.

In embodiments of the methods of genetic modification provided herein, at least 70%, 71%, 72%, 73%, 74%, 75%, 76%, 77%, 78%, 79%, 80%, 81%, 82%, 83%, 84%, 85%, 86%, 87%, 88%, 89, 90%, 91%, 92%, 93%, 94%, 95%, 96%, 97%, 98%, or at least 99% of the transfected cells are administered to a patient in need thereof, without selection or enrichment of the cells. In embodiments, at least 70% of the transfected cells are administered to a patient in need thereof, without selection or enrichment of the cells. In embodiments, at least 75% of the transfected cells are administered to a patient in need thereof, without selection or enrichment of the cells. In embodiments, at least 80% of the transfected cells are administered to a patient in need thereof, without selection or enrichment of the cells. In embodiments, at least 85% of the transfected cells are administered to a patient in need thereof, without selection or enrichment of the cells. In embodiments, at least 90% of the transfected cells are administered to a patient in need thereof, without selection or enrichment of the cells. In embodiments, at least 95% of the transfected cells are administered to a patient in need thereof, without selection or enrichment of the cells.

In embodiments of the methods of genetic modification provided herein, at least 70%, 71%, 72%, 73%, 74%, 75%, 76%, 77%, 78%, 79%, 80%, 81%, 82%, 83%, 84%, 85%, 86%, 87%, 88%, 89, 90%, 91%, 92%, 93%, 94%, 95%, 96%, 97%, 98%, or at least 99% of the transfected cells are used in a subsequent reaction, without selection or enrichment of the cells. In embodiments of the methods of genetic modification provided herein, at least 70% of the transfected cells are used in a subsequent reaction, without selection or enrichment of the cells. In embodiments of the methods of genetic modification provided herein, at least 75% of the transfected cells are used in a subsequent reaction, without selection or enrichment of the cells. In embodiments of the methods of genetic modification provided herein, at least 80% of the transfected cells are used in a subsequent reaction, without selection or enrichment of the cells. In embodiments of the methods of genetic modification provided herein, at least 85% of the transfected cells are used in a subsequent reaction, without selection or enrichment of the cells. In embodiments of the methods of genetic modification provided herein, at least 90% of the transfected cells are used in a subsequent reaction, without selection or enrichment of the cells. In embodiments of the methods of genetic modification provided herein, at least 95% of the transfected cells are used in a subsequent reaction, without selection or enrichment of the cells.

In an aspect is provided a method of genetically modifying a plurality of myeloid cells, including transfecting the myeloid cells with a gene editing reagent, wherein the myeloid cells are not transduced with a viral vector, and wherein the method does not include a selection step or enrichment step following myeloid cell transfection. In embodiments, the cells are transfected via electroporation. In embodiments, the cells are transfected via lipid-based transfection.

For the methods provided herein including embodiments thereof, the site of interest is modified in at least 70% of the plurality of myeloid cells. In embodiments, the site of interest is modified in at least 80% of the plurality of myeloid cells. In embodiments, the site of interest is modified in at least 85% of the plurality of myeloid cells. In embodiments, the site of interest is modified in at least 90% of the plurality of myeloid cells. In embodiments, the plurality of myeloid cells includes dendritic cells (DCs) and the site of interest is modified in at least 50% of the DCs. In embodiments, the viability of the plurality of myeloid cells after electroporation is at least 50%. In embodiments, the viability of the plurality of myeloid cells after electroporation is at least 60%. In embodiments, the viability of the plurality of myeloid cells after electroporation is at least 70%. In embodiments, the viability of the plurality of myeloid cells after electroporation is at least 80%. In embodiments, the viability of the plurality of myeloid cells after electroporation is at least 90%.

In embodiments, the gene editing reagent includes an RNA-guided nuclease. In embodiments, the RNA-guided nuclease is a CRISPR-Cas system. In embodiments, the CRISPR-Cas system includes a Cas9 or a Cas9 variant. In embodiments, the CRISPR-Cas system includes a Cas12, a Cascade, a Cas13, or a variant of each thereof. In embodiments, the gene editing reagent includes a CRISPR-Cas system including a Cas protein, a guide RNA, and optionally a donor DNA. In embodiments, he Cas protein and guide RNA are non-covalently associated. In embodiments, the Cas protein, guide RNA and donor DNA are non-covalently associated. In embodiments, the gene editing reagent includes transcription activator-like effector nuclease (TALEN), a zinc finger nuclease, or an Argonaut endonuclease.

### Methods of Treatment

In an aspect, provided herein are methods of treating a disease treatable with a myeloid cell. The methods include providing a genetically modified myeloid cell that has not been transformed with a virus, wherein the myeloid cell has been transfected with a gene editing reagent, and administering the myeloid cell to a patient in need thereof.

In embodiments, diseases treatable with a myeloid cell include cancers or diseases characterized by abberant or dysfunctional myeloid cells. In embodiments, the cancer is chronic myeloid leukemia. In embodiments, the cancer is acute myeloid leukemia. In embodiments, the disease is treatable with a myeloid cell. In embodiments, the disease is an autoimmunie pathology, neuropathology, a disease with an immunoregulatory component involving myeloid cells, myelodysplastic syndrome, or a myeloproliferative disorder. In embodiments, the disease is an autoimmune pathology. In embodiments, the autoimmune pathology is systemic lupus erythematosus, rheumatoid arthritis, an inflammatory bowel disorder, or multiple sclerosis. In embodiments, the autoimmune pathology is systemic lupus erythematosus. In embodiments, the autoimmune pathology is an inflammatory bowel disorder. In embodiments, the autoimmune pathology is multiple sclerosis. In embodiments, the disease is a neuropathology. In embodiments, the disease includes an immunoregulatory component involving myeloid cells. In embodiments, the disease is myelodysplastic syndrome. In embodiments, the disease is a myeloproliferative disorder.

In embodiments, methods of treating a disease treatable with a myeloid cell include administering a genetically modified myeloid cell provided herein including embodiments thereof. In embodiments, the myeloid cell has been transfected with a gene editing reagent according to any aspect or embodiment provided herein. In embodiments, the myeloid cell has been genetically modified to mutate and replace a gene that causes a disease. In embodiments, the myeloid cell has been genetically modified to mutate a gene that causes a disease. In embodiments, the myeloid cell has been genetically modified to replace a gene that causes a disease. In embodiments, the myeloid cell has been genetically modified for cell therapy.

In embodiments, the myeloid cells are primary myeloid cells. In embodiments, the myeloid cells are cultured myeloid cells.

In embodiments, the gene editing reagent includes an RNA-guided nuclease. In embodiments, the RNA-guided nuclease is a CRISPR-Cas system. In embodiments, the CRISPR-Cas system includes a Cas9 or a Cas9 variant. In embodiments, the CRISPR-Cas system includes a Cas9. In embodiments, the CRISPR-Cas system includes a Cas9 variant. In embodiments, the CRISPR-Cas system includes a Cas12, Cascade, a Cas13, or a variant of each thereof. In embodiments, the CRISPR-Cas system includes a Cas12. In embodiments, the CRISPR-Cas system includes Cascade. In embodiments, the CRISPR-Cas system includes a Cas13. In embodiments, the CRISPR-Cas system includes a Cas12 variant. In embodiments, the CRISPR-Cas system includes Cascade, wherein one or more components of the Cascade complex is a variant. In embodiments, the CRISPR-Cas system includes a a Cas13 variant.

For the methods provided herein, in embodiments, the gene editing reagent includes a CRISPR-Cas system including a Cas protein, a guide RNA, and optionally a donor DNA. In embodiments, the Cas protein and guide RNA are non-covalently associated. In embodiments, the Cas protein, guide RNA and donor DNA are non-covalently associated.

For the methods provided herein including embodiments thereof, the gene editing reagent includes transcription activator-like effector nuclease (TALEN), a zinc finger nuclease, or an Argonaut endonuclease. In embodiments, the gene editing reagent includes transcription activator-like effector nuclease (TALEN). In embodiments, the gene editing reagent includes a zinc finger nuclease. In embodiments, the gene editing reagent includes an Argonaut endonuclease.

For the methods provided herein, in embodiments, administering a genetically modified myeloid to a patient in need thereof comprises intravenous, intramuscular, intra-arterial, or intrathecal administration. In embodiments, administering comprises intravenous administration. In embodiments, administering comprises intramuscular administration. In embodiments, administering comprises intra-arterial administration. In embodiments, administering comprises intrathecal administration. In embodiments, administering includes intravenous, parenteral, intraperitoneal, intramuscular, intralesional, or subcutaneous administration. In embodiments, administering includes intravenous administration. In embodiments, administering includes parenteral administration. In embodiments, administering includes intraperitoneal administration. In embodiments, administering includes intramuscular administration. In embodiments, administering includes intralesional administration. In embodiments, administering includes subcutaneous administration.

### Systems

Provided herein are systems for efficiently generating genetically modified myeloid cells. The cells may be genetically modified to include mutated or knocked-out genes. The systems provided herein produce population-level gene knockouts or gene mutants in myeloid cells. Thus, in an aspect, provided herein are systems for genetically modifying a myeloid cell in the absence of a viral vector. The system includes a chamber compatible with transfection system, multiple myeloid cells within the chamber in a media compatible with electroporation, and at least one gene editing system designed to target at least one site of interest in the genome of myeloid cells.

In embodiments, the systems for genetically modifying a myeloid cell in the absence of a viral vector provided herein includes a chamber compatible with a transfection system. In embodiments, the transfection system includes an electroporation apparatus.

In embodiments, the systems for genetically modifying a myeloid cell in the absence of a viral vector provided herein include a plurality of myeloid cells within a chamber compatible with a transfection system. In embodiments, the plurality of myeloid cells is in a culture medium compatible with electroporation. In embodiments, the culture medium includes nutrients, growth factors, and/or antibiotics. In embodiments, the culture medium includes DMEM High Glucose, fetal bovine serum, GlutaMAX (Gibco) and penicillin/streptomycin. In embodiments, the myeloid cell is a primary myeloid cell or a cultured myeloid cell. In embodiments, the myeloid cell is a primary myeloid cell. In embodiments, the myeloid cell is a cultured myeloid cell. In embodiments, the myeloid cell is a monocyte, a macrophage, or a dendritic cell. In embodiments, the myeloid cell is a monocyte. In embodiments, the myeloid cell is a macrophage. In embodiments, the myeloid cell is a dendritic cell.

In embodiments, the systems for genetically modifying a myeloid cell in the absence of a viral vector provided herein include a myeloid cell that is not subjected to a selection step and/or an enrichment step after transfection.

In embodiments, the systems for genetically modifying a myeloid cell in the absence of a viral vector provided herein include a gene editing system that includes a gene editing reagent. In embodiments, the gene editing reagent includes an RNA-guided nuclease. In embodiments, the RNA-guided nuclease is a CRISPR-Cas system. In embodiments, the CRISPR-Cas system includes a Cas9 or a Cas9 variant. In embodiments, the CRISPR-Cas system includes a Cas12, a Cascade, a Cas13, or a variant of each thereof. In embodiments, the gene editing reagent includes transcription activator-like effector nuclease (TALEN), a zinc finger nuclease, or an Argonaut endonuclease.

In embodiments, the systems for genetically modifying a myeloid cell in the absence of a viral vector provided herein include where the cells are contacted with an electroporation enhancer during transfection. In embodiments, the electroporation enhancer is a carrier DNA. In embodiments, the electroporation enhancer is a carrier DNA, a single stranded DNA, a combination of single stranded and double stranded DNA, a polymeric additive, and/or an oligonucleotide. In embodiments, the electroporation enhancer is a single stranded DNA. In embodiments, the electroporation enhancer is a combination of single stranded and double stranded DNA. In embodiments, the electroporation enhancer is a polymeric additive. In embodiments, the electroporation enhancer is an oligonucleotide. In embodiments, the oligonucleotide is a TLR antagonist such as A151. In embodiments, the carrier DNA is a single-stranded DNA oligonucleotide. In embodiments, electroporation is enhanced by the use of one or more JAK2 inhibitors, e.g. when Cas mRNA is delivered into the cell.

For the systems provided herein, in embodiments, the myeloid cell is not activated prior to or during genetic modification. In embodiments, the myeloid cell is not activated prior to genetic modification. In embodiments, the myeloid cell is not activated during genetic modification.

### Compositions and Uses Thereof

In an aspect, provided herein are compositions including a plurality of myeloid cells in contact with a gene editing reagent, a transfection buffer, and an electroporation enhancer, wherein the composition does not include a viral vector. In embodiments, the compositions provided herein include the myeloid cells according to any aspect or embodiment described herein. In embodiments, the compositions provided herein include a gene editing reagent according to any aspect or embodiment described herein.

In embodiments, the compositions provided herein include a transfection buffer. In embodiments, the transfection buffer includes a salt, a divalent cation, and a buffering agent. In embodiments, salts include, for example, NaCl, KCl, and sodium succinate. In embodiments, divalent cations include, for example, magnesium and calcium. In embodiments, buffering agents include, for example, sodium phosphate and HEPES. In embodiments, the transfection buffer may include other agents such as, for example, mannitol and sodium lactobionate.

In embodiments, the compositions provided herein include an electroporation enhancer. In embodiments, the electroporation enhancer is a carrier DNA, a single stranded DNA, a combination of single stranded and double stranded DNA, a polymeric additive, and/or an oligonucleotide. In embodiments, the electroporation enhancer includes is a carrier DNA. In embodiments, the carrier DNA is a single-stranded DNA oligonucleotide. In embodiments, the electroporation enhancer is a single stranded DNA. In embodiments, the electroporation enhancer is a combination of single stranded and double stranded DNA. In embodiments, the electroporation enhancer is a polymeric additive. In embodiments, the electroporation enhancer is an oligonucleotide. In embodiments, the oligonucleotide is a TLR antagonist. In embodiments, TLR antagonist is A151. In embodiments, electroporation is enhanced by the use of one or more JAK2 inhibitors, e.g. when Cas mRNA is delivered into the cell.

For the compositions provided herein, in embodiments, the myeloid cells are cultured myeloid cells. In embodiments, for the compositions provided herein, the plurality of myeloid cells is a plurality of monocyte, macrophage, or dendritic cells. In embodiments, the plurality of myeloid cells is a plurality of monocyte cells. In embodiments, for the compositions provided herein, the plurality of myeloid cells is a plurality of macrophage cells. In embodiments, for the compositions provided herein, the plurality of myeloid cells is a plurality dendritic cells.

In embodiments, the gene editing reagent includes an RNA-guided nuclease. In embodiments, the RNA-guided nuclease is a CRISPR-Cas system. In embodiments, the CRISPR-Cas system includes a Cas9 or a Cas9 variant. In embodiments, the CRISPR-Cas system includes a Cas9. In embodiments, the CRISPR-Cas system includes a Cas9 variant. In embodiments, the CRISPR-Cas system includes a Cas12, a Cascade, a Cas13, or a variant of each thereof. In embodiments, the CRISPR-Cas system includes a Cas12. In embodiments, the CRISPR-Cas system includes a Cascade. In embodiments, the CRISPR-Cas system includes a a Cas13. In embodiments, the CRISPR-Cas system includes a Cas12 variant. In embodiments, the CRISPR-Cas system includes a Cascade variant. In embodiments, the CRISPR-Cas system includes a a Cas13 variant.

For the compositions provided herein, in embodiments, the gene editing reagent includes a CRISPR-Cas system including a Cas protein, a guide RNA, and optionally a donor DNA. In embodiments, the Cas protein and guide RNA are non-covalently associated. In embodiments, the Cas protein, guide RNA and donor DNA are non-covalently associated.

For the compositions provided herein, in embodiments, the gene editing reagent includes transcription activator-like effector nuclease (TALEN), a zinc finger nuclease, or an Argonaut endonuclease. In embodiments, the gene editing reagent includes transcription activator-like effector nuclease (TALEN). In embodiments, the gene editing reagent includes a zinc finger nuclease. In embodiments, the gene editing reagent includes an Argonaut endonuclease.

In embodiments, the RNA-guided nuclease includes a guide RNA and a ribonucleoprotein (RNP), wherein the ratio of guide RNA to is between 100:1 and 1:100. In embodiments, the RNA-guided nuclease includes a guide RNA and a ribonucleoprotein (RNP), wherein the ratio of guide RNA to is less than or equal to about 3:1. In embodiments, the RNA-guided nuclease includes a guide RNA and a ribonucleoprotein (RNP), wherein the ratio of guide RNA to ribonucleoprotein (RNP) is less than or equal to about 2: 1. In embodiments, the RNA-guided nuclease includes a guide RNA and a ribonucleoprotein (RNP), wherein the ratio of guide RNA to ribonucleoprotein (RNP) is about 3:1. In embodiments, the RNA-guided nuclease includes a guide RNA and a ribonucleoprotein (RNP), wherein the ratio of guide RNA to ribonucleoprotein (RNP) is about 2: 1. In embodiments, the electroporation enhancer is selected from a carrier DNA, a single stranded DNA, a combination of single stranded and double stranded DNA, a polymeric additive, and an oligonucleotide. In embodiments, the carrier DNA is a single-stranded DNA oligonucleotide.

The genetically modified cells provided herein are contemplated to be effective for treatment of diseases characterized by abberant or dysfunctional myeloid cells (e.g. chronic myeloid leukemia, acute myeloid leukemia etc.). Thus, in an aspect is provided a genetically modified myeloid cell provided herein including embodiments thereof. In another aspect is provided a genetically modified myeloid cell provided herein including embodiments thereof with a pharmaceutically acceptable excipient.

The genetically modified cells can further be used in methods for discovering and validating drugs for treatment of diseases characterized by abberant or dysfunctional myeloid cells. Thus, in an aspect is provided an assay for drug discovery including screening the effect of one or more compounds on a myeloid cell provided herein including embodiments thereof. In another aspect is provided a method for target validation of a compound, including contacting a myeloid cell provided herein including embodiments thereof with the compound and monitoring an effect on the cell.

### EMBODIMENTS

Embodiment 1: A method for genetic modification of a myeloid cell, the method comprising transfecting the myeloid cell with a gene editing reagent targeting a genetic site of interest, wherein the myeloid cell is not transduced with a viral vector.

Embodiment 2: The method of Embodiment 1, wherein the myeloid cell is a primary myeloid cell.

Embodiment 3: The method of Embodiment 1 or 2, wherein the myeloid cell is a monocyte, macrophage, neutrophil, basophil, eosinophil, erythrocyte, dendritic cell, or megakaryocyte.

Embodiment 4: The method of any one of Embodiments 1-3, wherein the cell is transfected via electroporation.

Embodiment 5: The method of any one of Embodiments 1-4, wherein the cell is transfected via nucleofection.

Embodiment 6: The method of any one of Embodiments 1-5, wherein the cell is transfected via lipid-based or polymer-based transfection.

Embodiment 7: The method of Embodiment 6, wherein the cell is transfected via lipid-based transfection.

Embodiment 8: The method of any one of Embodiments 1-7, wherein the cell is not subjected to a selection step and/or an enrichment step after transfection.

Embodiment 9: The method of any one of Embodiments 1-8, wherein the gene editing reagent comprises an RNA-guided nuclease.

Embodiment 10: The method of Embodiment 9, wherein the RNA-guided nuclease is a CRISPR-Cas system.

Embodiment 11: The method of Embodiment 10, wherein the CRISPR-Cas system comprises a Cas9 or a Cas9 variant.

Embodiment 12: The method of Embodiment 10, wherein the CRISPR-Cas system comprises a Cas12, a Cascade, a Cas13, or a variant of each thereof.

Embodiment 13: The method of any one of Embodiments 1-12, wherein the gene editing reagent comprises a CRISPR-Cas system comprising a Cas protein, a guide RNA, and optionally a donor DNA.

Embodiment 14: The method of Embodiment 13, wherein the Cas protein and guide RNA are non-covalently associated.

Embodiment 15: The method of Embodiment 13 or 14, wherein the Cas protein, guide RNA and donor DNA are non-covalently associated.

Embodiment 16: The method of any one of Embodiments 1-8, wherein the gene editing reagent comprises transcription activator-like effector nuclease (TALEN), a zinc finger nuclease, or an Argonaut endonuclease.

Embodiment 17: The method of any one of Embodiments 1-16, wherein the cells are contacted with an electroporation enhancer during transfection.

Embodiment 18: The method of Embodiment 17, wherein the electroporation enhancer is selected from a carrier DNA, a single stranded DNA, a combination of single stranded and double stranded DNA, a polymeric additive, and an oligonucleotide.

Embodiment 19: The method of Embodiment 18, wherein the carrier DNA is a single-stranded DNA oligonucleotide.

Embodiment 20: The method of Embodiment 18 or 19, wherein the carrier DNA is non-homologous to human, mouse, and/or rat genomes.

Embodiment 21: The method of any one of Embodiments 1-20, wherein the myeloid cell is differentiated prior to electroporation.

Embodiment 22: The method of Embodiment 21, wherein the myeloid cell is differentiated into a dendritic cell.

Embodiment 23: The method of Embodiment 21, wherein the myeloid cell is differentiated into a macrophage.

Embodiment 24: The method of any one of Embodiments 1-23, wherein the myeloid cell is not activated prior to or during genetic modification.

Embodiment 25: The method of any one of Embodiments 1-24, wherein two or more distinct crispr RNAs (crRNA) to the site of interest are introduced into the myeloid cell.

Embodiment 26: The method of Embodiment 25, wherein introducing the crRNAs into the myeloid cell comprises transfecting the cell with the crRNAs.

Embodiment 27: The method of Embodiment 25 or 26, wherein the crRNAs are contacted with tracrRNAs.

Embodiment 28: The method of Embodiment 27, wherein the crRNAs are annealed to the tracrRNAs.

Embodiment 29: The method of Embodiment 25 or 26, wherein the crRNAs are single guide RNAs (sgRNAs).

Embodiment 30: The method of any one of Embodiments 1-29, wherein multiple genetic sites of interest are targeted.

Embodiment 31: The method of Embodiment 30, wherein two or more distinct crRNAs to each site of interest are introduced into the myeloid cell.

Embodiment 32: The method of any one of Embodiments 1-31, wherein the myeloid cell is a human cell.

Embodiment 33: A method for genetic modification of a plurality of myeloid cells, the method comprising transfecting the plurality of myeloid cells in the presence of a gene editing reagent targeting a site of interest, wherein the myeloid cells are not transduced with a viral vector.

Embodiment 34: The method of Embodiment 33, wherein the myeloid cells are cultured myeloid cells.

Embodiment 35: The method of Embodiment 33 or 34, wherein the cell is transfected via electroporation.

Embodiment 36: The method of any one of Embodiments 33-35, wherein the cell is transfected via lipid-based transfection.

Embodiment 37: The method of any one of Embodiments 33-36, wherein the site of interest is modified in at least 70% of the plurality of myeloid cells.

Embodiment 38: The method of Embodiment 37, wherein the site of interest is modified in at least 80% of the plurality of myeloid cells.

Embodiment 39: The method of Embodiment 37 or 38, wherein the site of interest is modified in at least 85% of the plurality of myeloid cells.

Embodiment 40: The method of any one of Embodiments 37-39, wherein the site of interest is modified in at least 90% of the plurality of myeloid cells.

Embodiment 41: The method of any one of Embodiments 33-40, wherein the plurality of myeloid cells comprises dendritic cells (DCs) and the site of interest is modified in at least 50% of the DCs.

Embodiment 42: The method of any one of Embodiments 33-41, wherein the viability of the plurality of myeloid cells after electroporation is at least 80%.

Embodiment 43: The method of Embodiment 42, wherein the viability of the plurality of myeloid cells after electroporation is at least 90%.

Embodiment 44: The method of any one of Embodiments 33-43, wherein the gene editing reagent comprises an RNA-guided nuclease.

Embodiment 45: The method of Embodiment 44, wherein the RNA-guided nuclease is a CRISPR-Cas system.

Embodiment 46: The method of Embodiment 45, wherein the CRISPR-Cas system comprises a Cas9 or a Cas9 variant.

Embodiment 47: The method of Embodiment 45, wherein the CRISPR-Cas system comprises a Cas12, Cascade, a Cas13, or a variant of each thereof.

Embodiment 48: The method of any one of Embodiments 33-47, wherein the gene editing reagent comprises a CRISPR-Cas system comprising a Cas protein, a guide RNA, and optionally a donor DNA.

Embodiment 49: The method of Embodiment 48, wherein the Cas protein and guide RNA are non-covalently associated.

Embodiment 50: The method of Embodiment 48 or 49, wherein the Cas protein, guide RNA and donor DNA are non-covalently associated.

Embodiment 51: The method of any one of Embodiments 33-43, wherein the gene editing reagent comprises transcription activator-like effector nuclease (TALEN), a zinc finger nuclease, or an Argonaut endonuclease.

Embodiment 52: The method of Embodiments 9-13 or 44-47, wherein the RNA-guided nuclease comprises a guide RNA and a ribonucleoprotein (RNP), wherein the ratio of guide RNA to RNP is between 100:1 and 1:100.

Embodiment 53: The method of Embodiment 52, wherein the ratio of guide RNA to RNP is less than or equal to about 3:1.

Embodiment 54: The method of Embodiment 52, wherein the ratio of guide RNA to ribonucleoprotein (RNP) is less than or equal to about 2:1.

Embodiment 55: The method of Embodiment 52, wherein the ratio of guide RNA to ribonucleoprotein (RNP) is about 3:1.

Embodiment 56: The method of Embodiment 52, wherein the ratio of guide RNA to ribonucleoprotein (RNP) is about 2:1.

Embodiment 57: The method of any one of Embodiments 1-56, wherein at least 70% of the transfected cells are administered to a patient in need thereof, without selection or enrichment of the cells.

Embodiment 58: The method of any one of Embodiments 1-57, wherein at least 70% of the transfected cells are used in a subsequent reaction, without selection or enrichment of the cells.

Embodiment 59: A method of genetically modifying a plurality of myeloid cells, comprising transfecting the myeloid cells with a gene editing reagent, wherein the myeloid cells are not transduced with a viral vector, and wherein the method does not comprise a selection step or enrichment step following myeloid cell transfection.

Embodiment 60: The method of Embodiment 59, wherein the cells are transfected via electroporation.

Embodiment 61: The method of Embodiment 59, wherein the cells are transfected via lipid-based transfection.

Embodiment 62: The method of any one of Embodiments 59-61, wherein the site of interest is modified in at least 70% of the plurality of myeloid cells.

Embodiment 63: The method of Embodiment 62, wherein the site of interest is modified in at least 80% of the plurality of myeloid cells.

Embodiment 64: The method of Embodiment 62 or 63, wherein the site of interest is modified in at least 85% of the plurality of myeloid cells.

Embodiment 65: The method of any one of Embodiments 62-64, wherein the site of interest is modified in at least 90% of the plurality of myeloid cells.

Embodiment 66: The method of any one of Embodiments 59-61, wherein the plurality of myeloid cells comprises dendritic cells (DCs) and the site of interest is modified in at least 50% of the DCs.

Embodiment 67: The method of any one of Embodiments 59-66, wherein the viability of the plurality of myeloid cells after electroporation is at least 80%.

Embodiment 68: The method of Embodiment 67, wherein the viability of the plurality of myeloid cells after electroporation is at least 90%.

Embodiment 69: The method of any one of Embodiments 59-68, wherein the gene editing reagent comprises an RNA-guided nuclease.

Embodiment 70: The method of Embodiment 69, wherein the RNA-guided nuclease is a CRISPR-Cas system.

Embodiment 71: The method of Embodiment 70, wherein the CRISPR-Cas system comprises a Cas9 or a Cas9 variant.

Embodiment 72: The method of Embodiment 70, wherein the CRISPR-Cas system comprises a Cas12, a Cascade, a Cas13, or a variant of each thereof.

Embodiment 73: The method of any one of Embodiments 59-72, wherein the gene editing reagent comprises a CRISPR-Cas system comprising a Cas protein, a guide RNA, and optionally a donor DNA.

Embodiment 74: The method of Embodiment 73, wherein the Cas protein and guide RNA are non-covalently associated.

Embodiment 75: The method of Embodiment 73 or 74, wherein the Cas protein, guide RNA and donor DNA are non-covalently associated.

Embodiment 76: The method of any one of Embodiments 59-68, wherein the gene editing reagent comprises transcription activator-like effector nuclease (TALEN), a zinc finger nuclease, or an Argonaut endonuclease.

Embodiment 77: A system for genetically modifying a myeloid cell in the absence of a viral vector, the system comprising a chamber compatible with a transfection system, multiple myeloid cells within the chamber in a media compatible with electroporation, and at least one gene editing system designed to target at least one site of interest in the genome of myeloid cells.

Embodiment 78: The system of Embodiment 77, further comprising a transfection system.

Embodiment 79: The system of Embodiment 77 or 78, wherein the transfection system comprises an electroporation apparatus.

Embodiment 80: The system of any one of Embodiments 77-79, wherein the myeloid cell is a primary myeloid cell.

Embodiment 81: The system of any one of Embodiments 77-79, wherein the myeloid cell is a cultured myeloid cell.

Embodiment 82: The system of any one of Embodiments 77-81, wherein the myeloid cell is a monocyte, a macrophage, or a dendritic cell.

Embodiment 83: The system of any one of Embodiments 77-82, wherein the cell is not subjected to a selection step and/or an enrichment step after transfection.

Embodiment 84: The system of any one of Embodiments 77-83, wherein the gene editing system comprises a gene editing reagent.

Embodiment 85: The system of Embodiment 84, wherein the gene editing reagent comprises an RNA-guided nuclease.

Embodiment 86: The system of Embodiment 85, wherein the RNA-guided nuclease is a CRISPR-Cas system.

Embodiment 87: The system of Embodiment 85, wherein the CRISPR-Cas system comprises a Cas9 or a Cas9 variant.

Embodiment 88: The system of Embodiment 86, wherein the CRISPR-Cas system comprises a Cas12, a Cascade, a Cas13, or a variant of each thereof.

Embodiment 89: The method of any one of Embodiments 84-88, wherein the gene editing reagent comprises a CRISPR-Cas system comprising a Cas protein, a guide RNA, and optionally a donor DNA.

Embodiment 90: The method of Embodiment 89, wherein the Cas protein and guide RNA are non-covalently associated.

Embodiment 91: The method of Embodiment 89 or 90, wherein the Cas protein, guide RNA and donor DNA are non-covalently associated.

Embodiment 92: The system of Embodiment 84, wherein the gene editing reagent comprises transcription activator-like effector nuclease (TALEN), a zinc finger nuclease, or an Argonaut endonuclease.

Embodiment 93: The system of any one of Embodiments 77-92, wherein the cells are contacted with an electroporation enhancer during transfection.

Embodiment 94: The system of Embodiment 93, wherein the electroporation enhancer is selected from a carrier DNA, a single stranded DNA, a combination of single stranded and double stranded DNA, a polymeric additive, and an oligonucleotide.

Embodiment 95: The system of Embodiment 94, wherein the carrier DNA is a single-stranded DNA oligonucleotide.

Embodiment 96: The system of any one of Embodiments 77-95, wherein the myeloid cell is not activated prior to or during genetic modification.

Embodiment 97: A method of treating a disease treatable with a myeloid cell, comprising providing a genetically modified myeloid cell that has not been transduced with a virus, wherein the myeloid cell has been transfected with a gene editing reagent; and administering the myeloid cell to a patient in need thereof.

Embodiment 98: The method of Embodiment 97, wherein the myeloid cells are primary myeloid cells.

Embodiment 99: The method of Embodiment 97, wherein the myeloid cells are cultured myeloid cells.

Embodiment 100: The method of any one of Embodiments 97-99, wherein the gene editing reagent comprises an RNA-guided nuclease.

Embodiment 101: The method of Embodiment 100, wherein the RNA-guided nuclease is a CRISPR-Cas system.

Embodiment 102: The method of Embodiment 101, wherein the CRISPR-Cas system comprises a Cas9 or a Cas9 variant.

Embodiment 103: The method of Embodiment 101, wherein the CRISPR-Cas system comprises a Cas12, a Cascade, a Cas13, or a variant of each thereof.

Embodiment 104: The method of any one of Embodiments 97-103, wherein the gene editing reagent comprises a CRISPR-Cas system comprising a Cas protein, a guide RNA, and optionally a donor DNA.

Embodiment 105: The method of Embodiment 104, wherein the Cas protein and guide RNA are non-covalently associated.

Embodiment 106: The method of Embodiment 104 or 105, wherein the Cas protein, guide RNA and donor DNA are non-covalently associated.

Embodiment 107: The method of any one of Embodiments 97-99, wherein the gene editing reagent comprises transcription activator-like effector nuclease (TALEN), a zinc finger nuclease, or an Argonaut endonuclease.

Embodiment 108: The method of any one of Embodiments 97-107, wherein administering comprises oral, intravenous, parenteral, intraperitoneal, intramuscular, intralesional, or subcutaneous administration.

Embodiment 109: A composition comprising a plurality of myeloid cells, a gene editing reagent, a transfection buffer, and an electroporation enhancer, wherein the composition does not comprise a viral vector.

Embodiment 110: The composition of Embodiment 109, wherein the myeloid cells are cultured myeloid cells.

Embodiment 111: The composition of Embodiment 109 or 110, wherein the plurality of myeloid cells is a plurality of monocyte, macrophage, or dendritic cells.

Embodiment 112: The composition of any one of Embodiments 109-111, wherein the gene editing reagent comprises an RNA-guided nuclease.

Embodiment 113: The composition of Embodiment 112, wherein the RNA-guided nuclease is a CRISPR-Cas system.

Embodiment 114: The composition of Embodiment 113, wherein the CRISPR-Cas system comprises a Cas9 or a Cas9 variant.

Embodiment 115: The composition of Embodiment 113, wherein the CRISPR-Cas system comprises a Cas12, a Cascade, a Cas13, or a variant of each thereof.

Embodiment 116: The composition of any one of Embodiments 109-115, wherein the gene editing reagent comprises a CRISPR-Cas system comprising a Cas protein, a guide RNA, and optionally a donor DNA.

Embodiment 117: The composition of Embodiment 116, wherein the Cas protein and guide RNA are non-covalently associated.

Embodiment 118: The composition of Embodiment 116 or 117, wherein the Cas protein, guide RNA and donor DNA are non-covalently associated.

Embodiment 119: The composition of any one of Embodiments 109-112, wherein the gene editing reagent comprises transcription activator-like effector nuclease (TALEN), a zinc finger nuclease, or an Argonaut endonuclease.

Embodiment 120: The composition of any one of Embodiments 112-115, wherein the RNA-guided nuclease comprises a guide RNA and a ribonucleoprotein (RNP), wherein the ratio of guide RNA to is between 100:1 and 1:100.

Embodiment 121: The composition of any one of Embodiments 112-115, wherein the RNA-guided nuclease comprises a guide RNA and a ribonucleoprotein (RNP), wherein the ratio of guide RNA to is less than or equal to about 3:1.

Embodiment 122: The composition of any one of Embodiments 112-115, wherein the RNA-guided nuclease comprises a guide RNA and a ribonucleoprotein (RNP), wherein the ratio of guide RNA to ribonucleoprotein (RNP) is less than or equal to about 2:1.

Embodiment 123: The composition of any one of Embodiments 112-115, wherein the RNA-guided nuclease comprises a guide RNA and a ribonucleoprotein (RNP), wherein the ratio of guide RNA to ribonucleoprotein (RNP) is about 3:1.

Embodiment 124: The composition of any one of Embodiments 112-115, wherein the RNA-guided nuclease comprises a guide RNA and a ribonucleoprotein (RNP), wherein the ratio of guide RNA to ribonucleoprotein (RNP) is about 2:1.

Embodiment 125: The composition of any one of Embodiments 109-124, wherein the electroporation enhancer is selected from a carrier DNA, a single stranded DNA, a combination of single stranded and double stranded DNA, a polymeric additive, and an oligonucleotide.

Embodiment 126: The composition of Embodiment 125, wherein the carrier DNA is a single-stranded DNA oligonucleotide.

Embodiment 127: A genetically modified myeloid cell made by the method of any one of claims [0363] 1 to [0363] 76.

Embodiment 128: An assay for drug discovery comprising screening the effect of one or more compounds on the myeloid cell of Embodiment 127.

Embodiment 129: A method for target validation of a compound, comprising contacting a myeloid cell of Embodiment 127 with the compound and monitoring an effect on the cell.

### EXAMPLES

One skilled in the art would understand that descriptions of making and using the cells or compositions described herein is for the sole purpose of illustration, and that the present disclosure is not limited by this illustration.

### Example 1: Efficient Genome Engineering in Murine Bone-marrow Derived Macrophage And Dendritic Cells

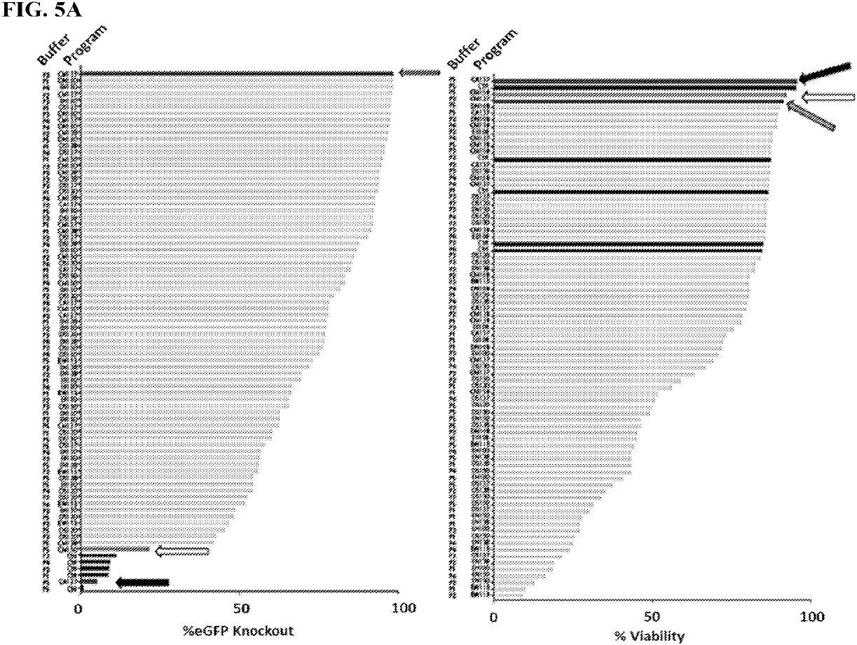

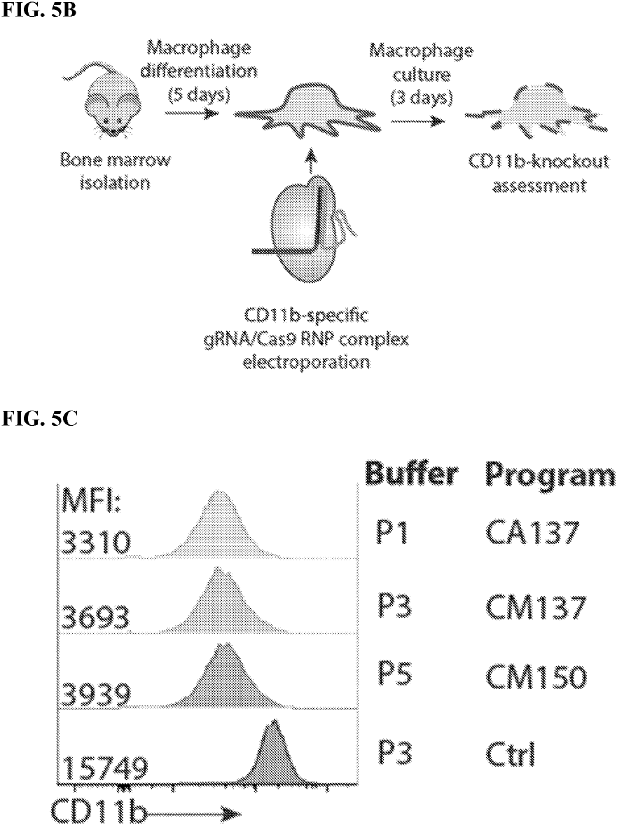

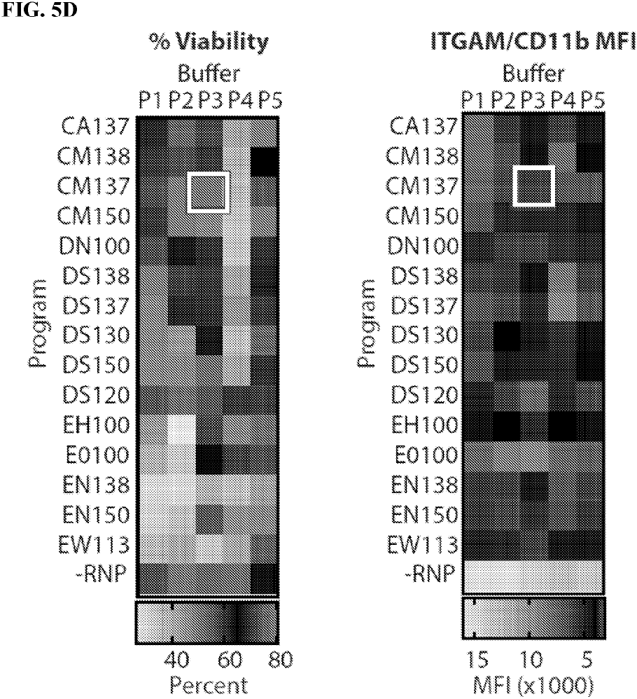

Macrophages are primary sensors of infection and tissue injury, with the ability to either promote inflammation, cellular and humoral immunity, or drive tissue regeneration and potentially fibrosis (Wynn and Vannella, 2016). Murine bone marrow-derived macrophages (BMDMs) are derived from hematopoietic progenitors by culture of total bone marrow in the presence of CSF1/M-CSF, which generates large numbers of these cells for experimental use. BMDMs have proven invaluable in studying the function of macrophages and are widely used to understand innate immune signaling. An 80-condition flow-cytometry (FACS) based screen in murine BMDMs was performed to identify optimal electroporation-based condition(s) for gene disruption, using integrin CD11b (encoded by Itgam) as a model target locus (FIGS. 5A-C).

Five different buffers and 15 electroporation conditions were compared to identify which protocols provided high efficiency of gene editing while maintaining cell viability. BMDMs were differentiated for 5 days in M-CSF, then used at 0.5 million cells per electroporation condition. Two distinct crRNAs were designed to target the CD11b coding sequence and synthesized at Integrated DNA Technologies (IDT). Subsequently, these guides were annealed to tracrRNAs, complexed with Streptococcus pyogenes Cas9 (SpCas9, hereafter termed Cas9) protein (IDT Cas9 V2) and pooled to improve gene deletion probability. A 3:1 molar ratio of gRNA:Cas9 was used as a starting condition (60 pmol or 10 µg Cas9 per reaction), as previously reported for lymphocytes (Seki and Rutz, 2018). Following electroporation, BMDMs were cultured in M-CSF for an additional 5 days. Viability and CD11b deletion efficiency were compared by flow cytometry (FACS, FIGS. 5B, 5C). While a majority of electroporation conditions proved to be toxic (FIG. 5B), we identified several conditions which preserved cell viability while inducing CD11b disruption (FIG. 5C). Of these, the top 3 conditions (FIGS. 5C, 5D) revealed comparable loss of CD11b expression with buffer P3, program CM137 maintaining the highest level of cell viability (white box, FIG. 5D).

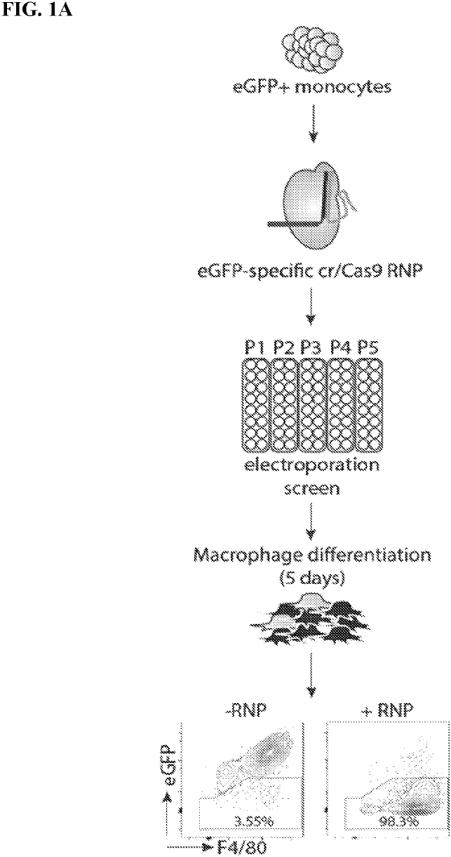

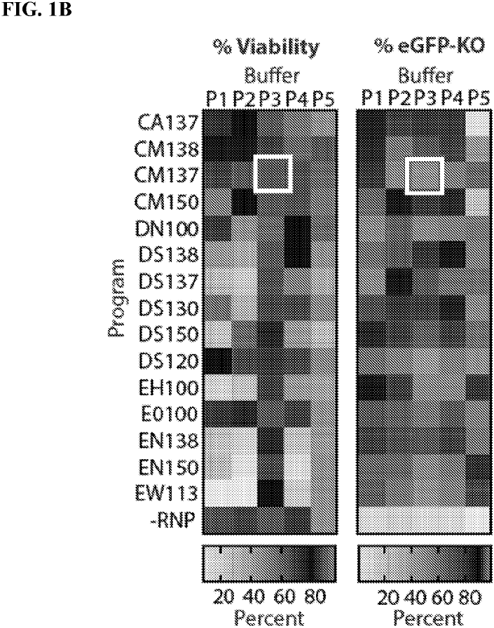

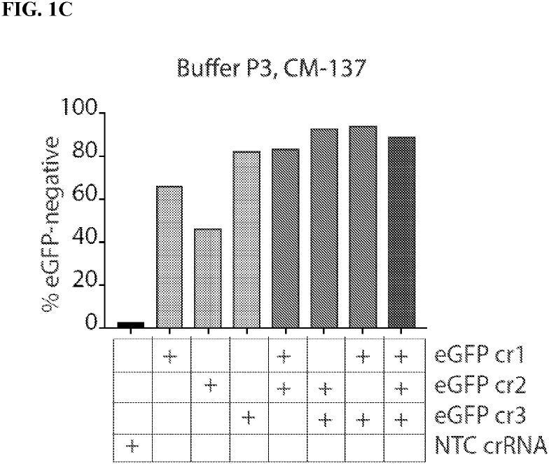

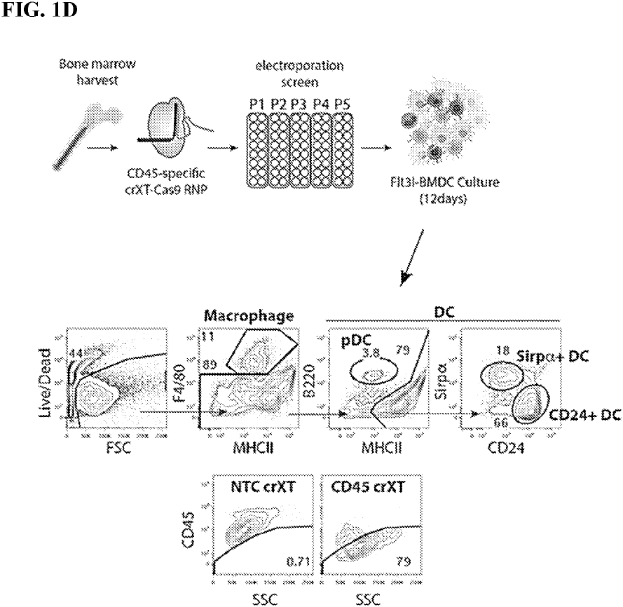

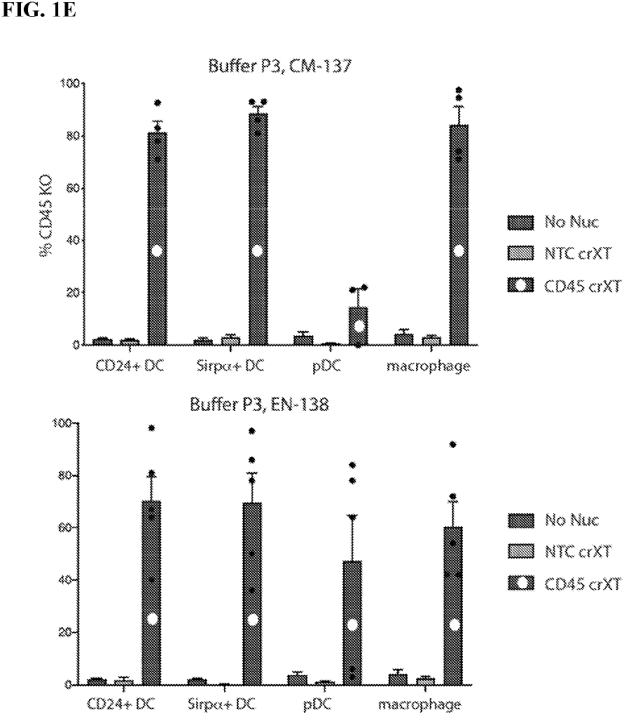

Next, a similar study was performed using monocytes isolated from femoral bone marrow of eGFP-transgenic mice, this time using distinct crRNA-tracrRNA sequences targeting egfp (Chen et al., 2017), to assess whether population-level gene deletion can be obtained from primary myeloid cell subsets (FIG. 1A). The ability to screen for loss of an intracellular marker such as eGFP precluded receptor internalization as a potential confounder in the previous assay. After Cas9/RNP electroporation, monocytes were cultured in M-CSF for 5 days to generate monocyte-derived macrophages. Consistent with findings for CD11b disruption, buffer P3, program CM137 again produced the greatest loss of target (eGFP) expression while maintaining cell viability (white boxes, FIG. 1B, ranking depicted in FIG. 5D). Next, these conditions were used to confirm the benefit of pooling multiple guide RNAs towards enhancement of target gene disruption. For example, egfp-targeting sequences g1 and g3 were delivered as a pool for the monocyte eGFP screen. Shown in FIG. 1C, individual gRNAs targeting egfp conferred varying degrees of gene loss; however, pooling g3 with either g1 or g2 provided maximal eGFP loss. Combining all 3 gRNAs did not improve eGFP loss beyond the most effective pairings. Thus, it was concluded that pooling two distinct crRNA-tracrRNAs targeting the same gene provides optimal gene KO in murine BMDMs or monocyte-derived macrophages.

Dendritic cells (DCs) play a critical role for instructing T cell responses through the process of antigen presentation (reviewed in (Merad et al., 2013)). Therefore, experiments were conducted to extend the protocol to genetic modification of DCs. Bone marrow-derived dendritic cells (BMDCs) serve as a model for investigating fundamental mechanisms of DC biology, and, more broadly, engineered DCs may constitute the critical material for future cell-based vaccines. A traditional method of differentiating bone marrow cells into DCs involves supplementation with GM-CSF (Cornel et al., 2018; Gundry et al., 2016). However, this does not produce the different physiologically relevant subsets found in vivo. Therefore, a more recently developed protocol was used that employs Flt3 ligand supplementation to differentiate bone marrow cells into at least three DC cell types found in vivo: plasmacytoid DC (pDC, B220+) cells and two conventional DC (cDC) cell types (Sirpα+ and CD24+) (Naik et al., 2005). In addition, this differentiation method allows the production of a large number of BMDCs, which are otherwise rare in vivo. Both plasmid transfection and viral transduction methods have been developed for primary DC gene delivery but, aside from variable and incomplete delivery across the target cell population, these methods require the use of biohazardous material and/or may introduce potential cell toxicity or differentiation artifacts (Bowles et al., 2011).

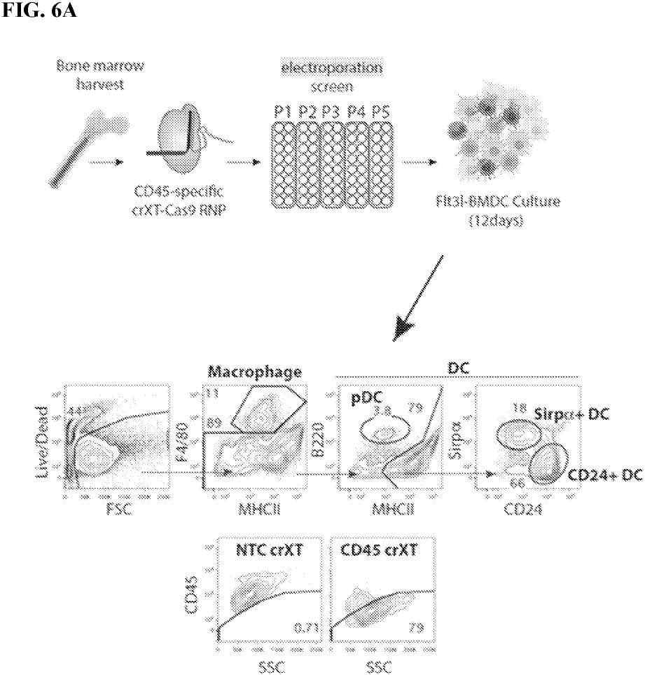

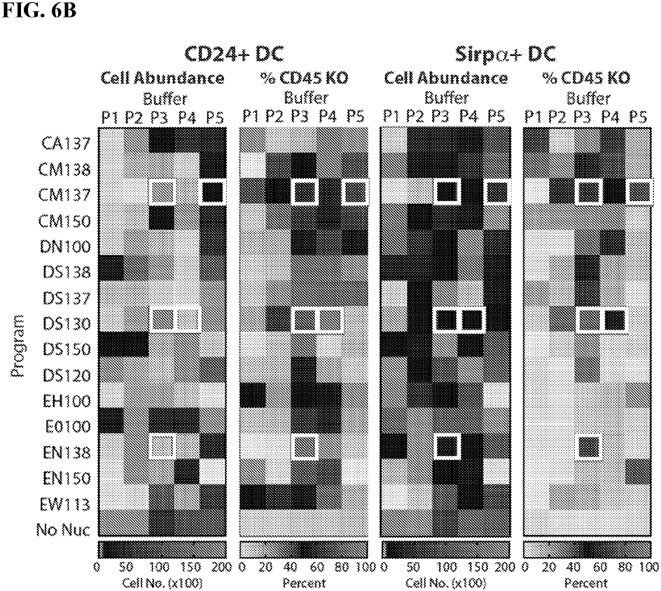

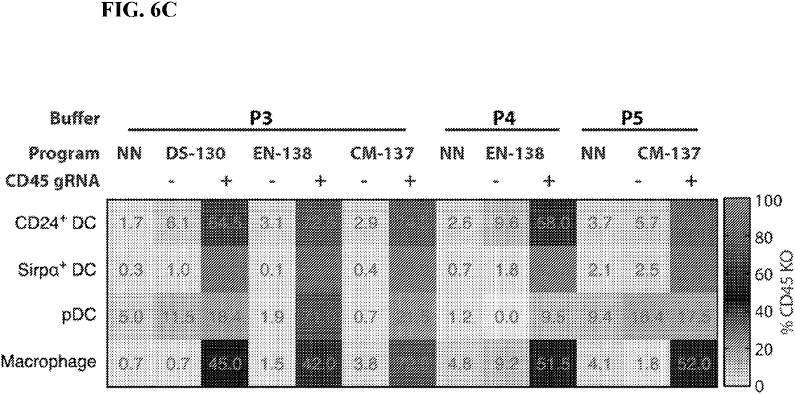

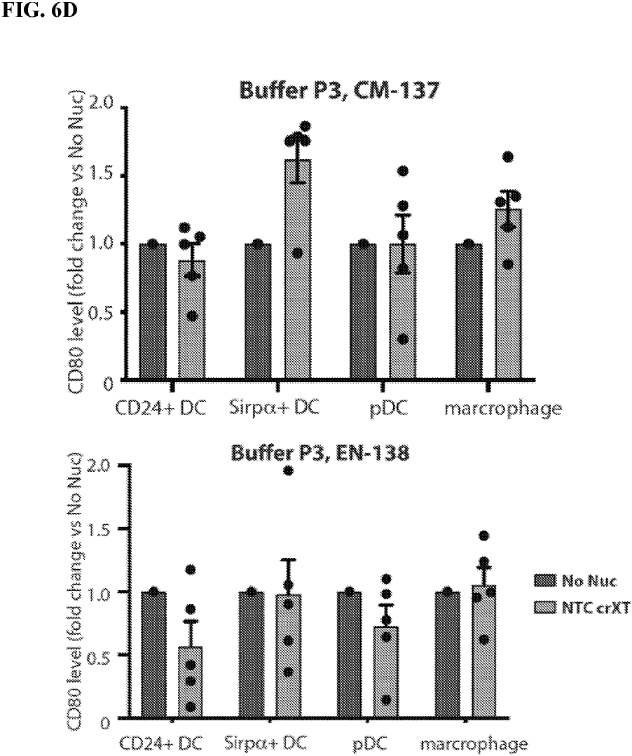

In order to adapt the protocol for DC gene editing, first delivery of the Cas9 RNPs was assessed. Mimicking the BMDM and monocyte workflows, a primary cell optimization screen in murine BMDCs, with the identical 5 buffer and 15 electroporation program combinations was performed (FIG. 6A). During the course of these studies, an improved guide RNA chemistry for the crRNA, termed crRNAXT, was made available from IDT. The XT-modified crRNAs were expected to improve the stability of the RNP and thus knockout efficiency. Accordingly, a single CD45-specific targeting sequence with this modification was used for the BMDC optimization screen. Bone marrow cells were isolated and 2 million cells per condition were evaluated which was determined to be the minimum number required for viability post-electroporation. A 3:1 molar ratio of gRNA:Cas9 was maintained but half the amount was used as compared to the BMDM and monocyte screens in order to increase the dynamic range for measuring improved knockout efficiency. Thus, 30 pmol or 5 µg Cas9 was used. Following RNP electroporation, cells were cultured for 12 days with Flt3 ligand (FIG. 6A). cDCs were isolated by FACS, and cell-surface CD45 expression was quantified by antibody stain (FIG. 6A). As in the macrophage screen, substantial variability was observed in both KO efficiency and viability for both the CD24+ DC and Sirpα+ DC populations across the eighty conditions tested, with CD24+ DCs displaying lower cell viability across the majority of conditions compared to Sirpα+ DCs (FIG. 6A).

Then, 5 conditions that resulted in substantial KO and acceptable viability were selected: Buffer P3 with program CM-137, DS130, EN-138, buffer P4 with program DS-130, and buffer P5 with program CM137 (white boxes, FIG. 5D; FIG. 6B). These conditions were tested with the higher Cas9/RNP concentration used in the macrophage screen, and this revealed that the combination of buffer P3 with program CM-137 (P3, CM137) gave the highest KO for CD24+ DCs (74.5% KO), Sirpα+ DCs (93% KO), and macrophages (72.5% KO) (FIG. 1E). However, this combination generated a relatively poor KO efficiency within pDCs (21.5%). Instead, results showed that buffer P3 with program EN-138 (P3, EN-138) was the most effective for the pDC subset (71% KO). Importantly, although this condition was not as broadly efficacious as buffer P3 with program CM-137, it still resulted in >50% KO across all tested DC cell types. Consistent with these results, when tested in cells from a distinct murine donor, P3, CM-137 showed high KO efficiency (>80%) in CD24+ DCs, Sirpα+ DCs, and macrophages, but not pDCs, while P3, EN-138 resulted in higher KO efficiency in pDCs (>50%) but displayed slightly lower and more variable KO efficiency across the other three cell types (FIG. 1E). Therefore, it was concluded that P3, CM-137 is the best condition for achieving KO in all DCs other than the pDC subtype, for which P3, EN-138 provides the highest KO efficiency.

Since it can be important to maintain the normal cell state as part of the gene editing process, expression levels of CD80, a co-stimulatory protein and myeloid cell activation marker, were tested at day 12 post-electroporation with our two selected conditions. Results showed that, with the exception of Sirpα+ DCs, the expression of CD80 was similar between negative controls and cells that had been electroporated using either condition, suggesting that delivery of Cas9/RNP by these methods does not trigger broad activation of murine bone marrow-derived myeloid populations (FIG. 6C).

### Example 2: Population-Level Gene Disruption in Human Monocyte Derived Dendritic Cells and Macrophages

Few methods are available for effective, non-viral gene modification of primary human macrophages and dendritic cells, which restricts direct phenotypic analysis of genes shared across species, the study of genes unique to humans, or, potentially, ex vivo editing of these populations for direct therapeutic benefit. Encouraged by results in murine monocyte-derived macrophage and dendritic cells (Example 1), similar conditions were attempted for delivery of Cas9/RNPs in human cells. In addition, expansion of the findings from murine cells was sought by comparing updated Cas9/RNP technology platforms in the human context. This included Cas9 proteins (sourced from different vendors) and guide RNA variants.

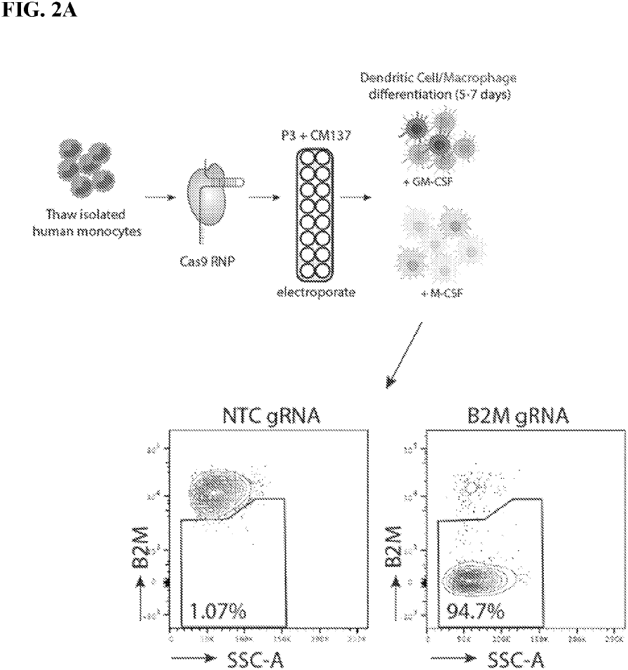

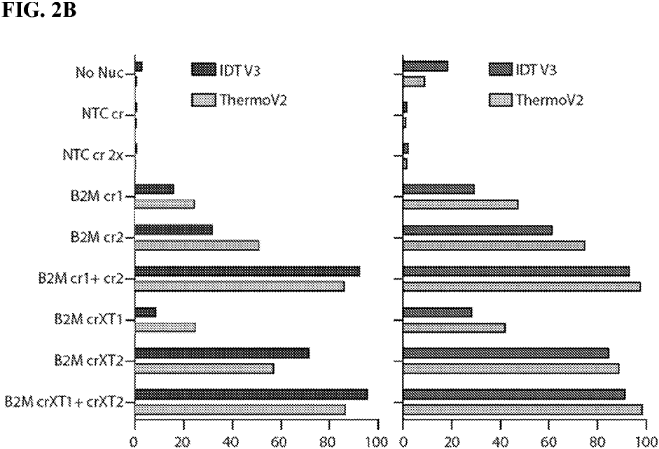

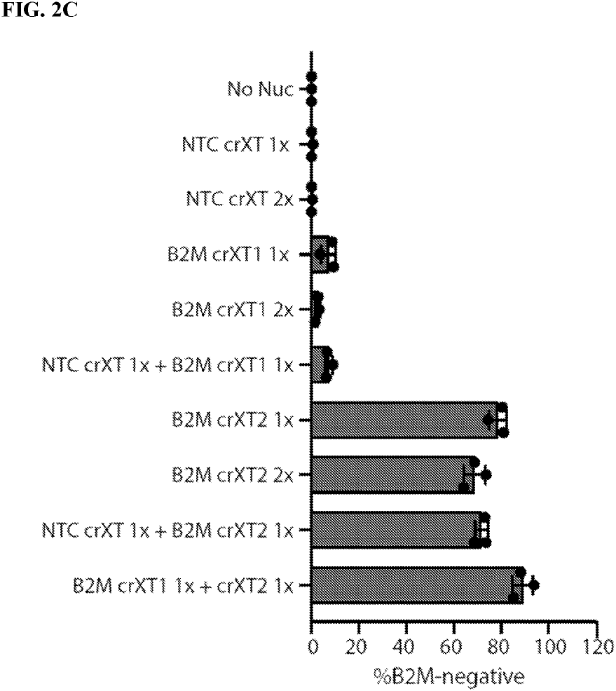

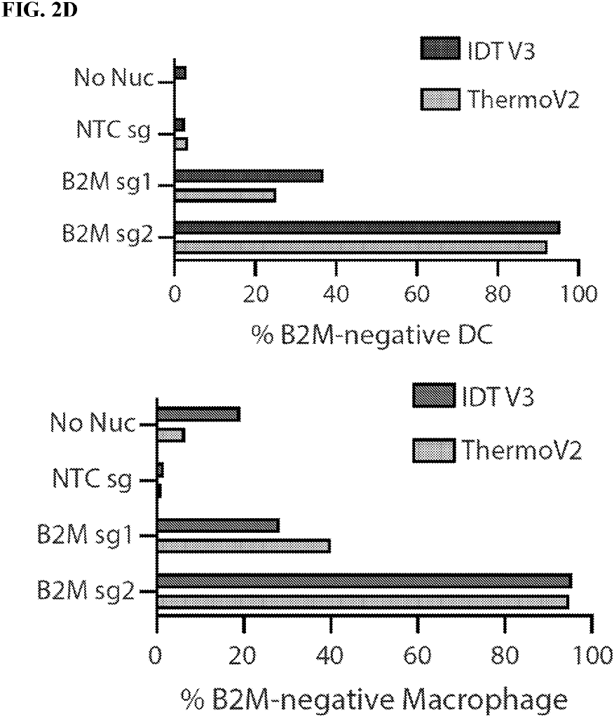

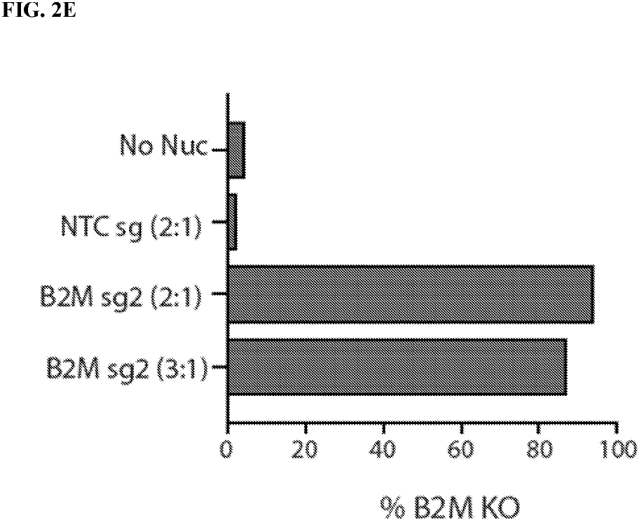

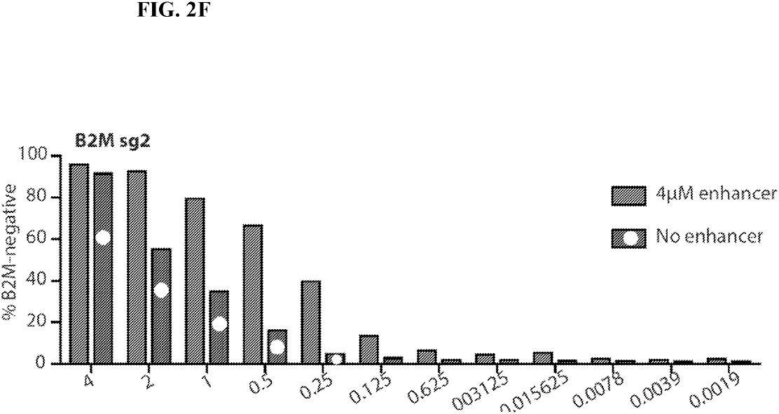

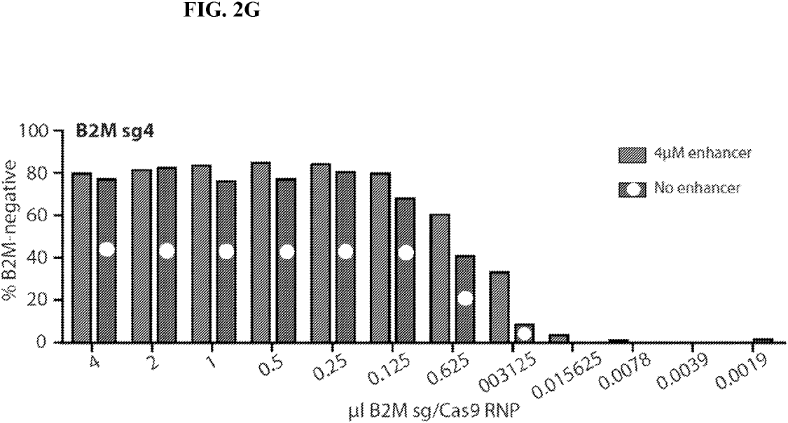

To begin optimization of human myeloid cell editing, β2-microglobulin (B2M), the broadly expressed constant region of the human MHCI complex was targeted. This would permit assessment of gene deletion across multiple donors regardless of genetic background. Several RNP variants loaded with two distinct guide sequences (termed B2M gRNA 1 or 2) were generated using standard or XT guide chemistry, alone or combined in each reaction, comprising either the “V3” Cas9 protein from Integrated DNA Technologies or “V2” from ThermoFisher, Inc. All RNPs were delivered into monocytes obtained from peripheral blood mononuclear cells (PBMCs) using buffer P3, CM-137, the generalizable condition identified in the murine assays (FIG. 2A). After electroporation of the various B2M-specific Cas9/guide RNA RNP variants, monocytes were differentiated into either macrophages (growth media supplemented with M-CSF, ~5 day culture) or dendritic cells (growth media supplemented with GM-CSF and IL-4, ~7 day culture). Results showed that the unique B2M gRNA sequences exhibited different KO efficiencies, regardless of guide chemistry, with gRNA2 cutting more efficiently than gRNA1, and KO was generally higher in the macrophage subset. While the individual guide efficacy neared 90% population-wide KO, the Cas9 RNP pools were able to induce near-complete KO (>90%) in both cell types (FIG. 2B). As with the individual guides, the standard and XT pools produced similar results in these assays. Negligible differences were observed when comparing the Cas9 protein variant performance across all tested conditions.

### Example 3: Cas9 RNPs Show Additivity When Targeting Two Guides to the Same Gene

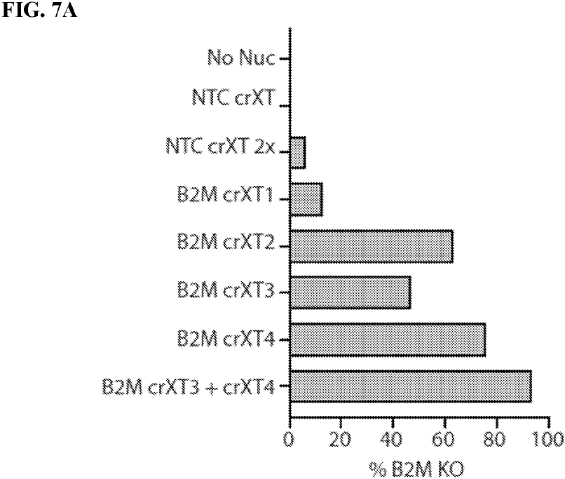

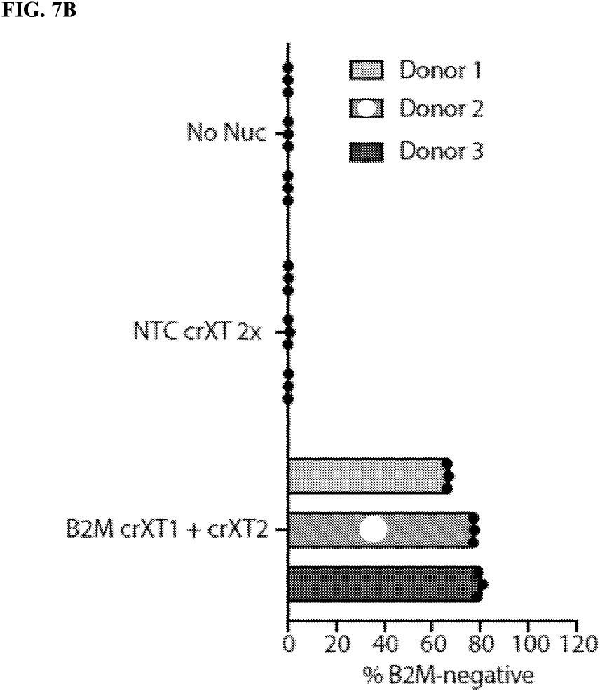

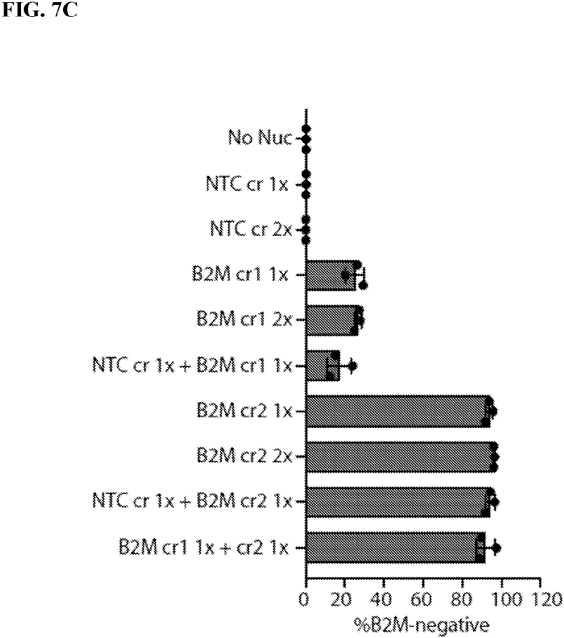

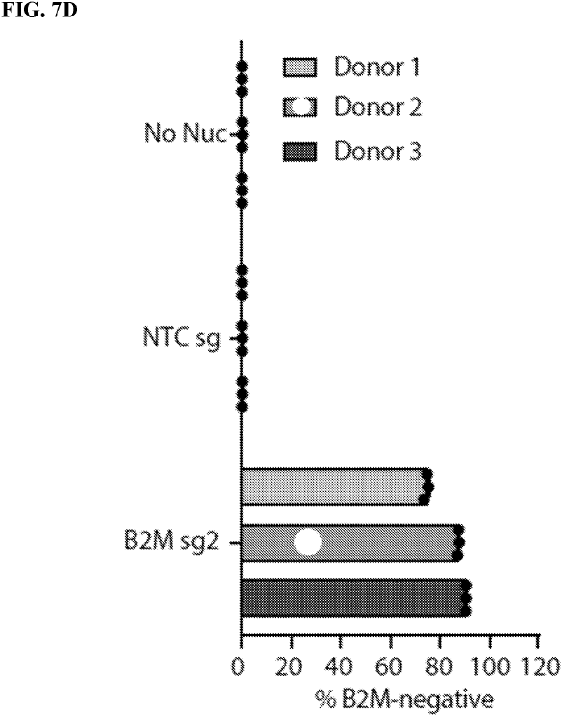

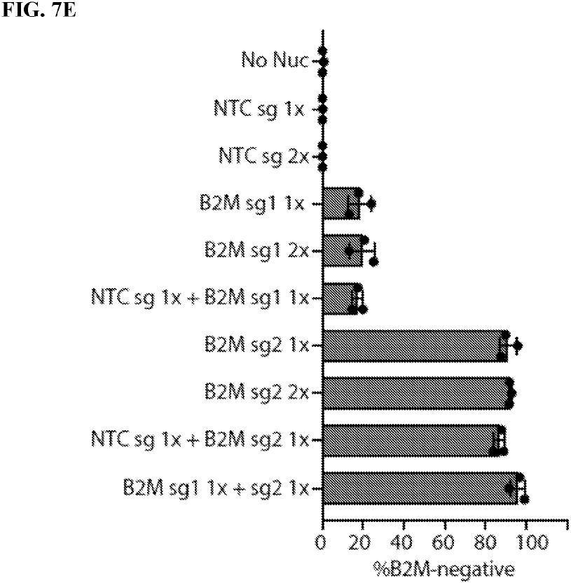

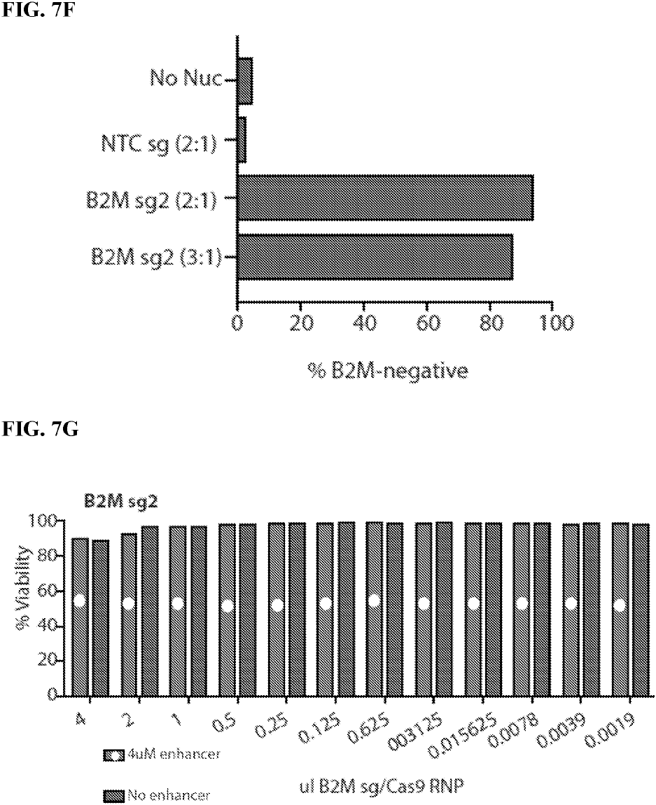

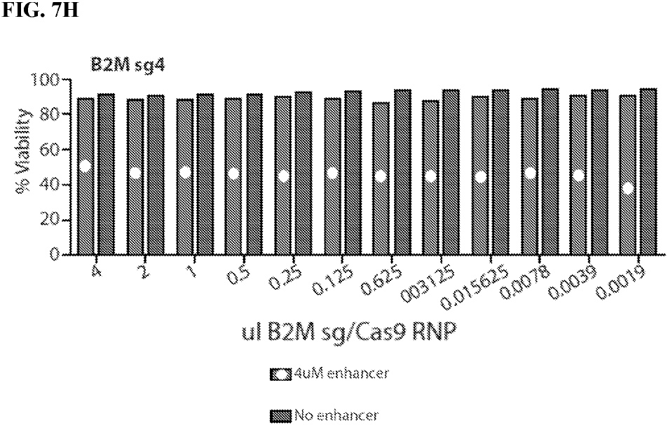

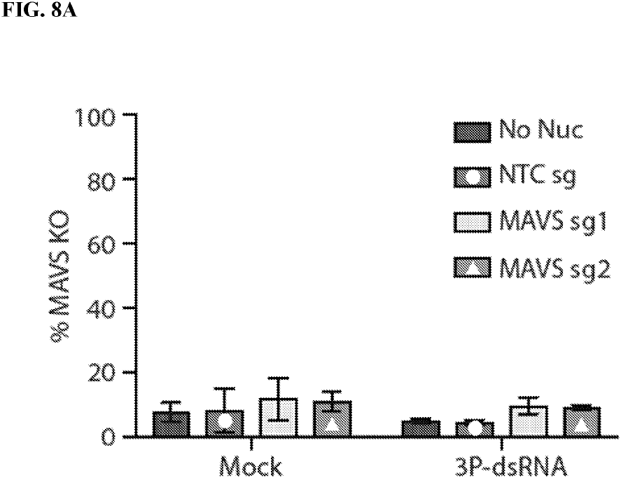

Across all mouse or human myeloid cell types that were tested through the initial optimization process, an apparent additive effect was routinely observed on gene disruption by combining multiple, unique guide RNAs against the same target, regardless of guide chemistry. For example, this was observed in human cells for B2M-specific gRNA sequences 1 and 2, as well as a second set of B2M guides (sequences 3 and 4, FIG. 7A), and this effect was independent of the human donor (FIG. 7B). Therefore, this relationship was studied in more detail.

Guide RNA pools were created by mixing equal parts of RNPs loaded with the unique guides prior to electroporation, effectively generating a 2X stock when compared to the individual guide preparations. While multiple targeting events may increase the probability of a frameshifting indel or a local chromosomal rearrangement, improving KO efficiency, it remains possible that the additive effect we observed with multiple guides was due to an elevated Cas9/RNP load introduced during the electroporation step. To test whether RNP load was the determining factor, delivery of 1X and 2X amounts of RNP loaded with individual XT guides targeting B2M in human cells was compared. As shown in FIG. 2C, no appreciable difference in KO efficiency was observed for either guide 1 (1x: 32.5% or 2x: 31.3%) or guide 2 (1x: 82.5% or 2x: 86%), suggesting the Cas9 protein amount was not limiting in the 1X condition.

Separately, we sought to evaluate whether the guide RNAs act independently or cooperatively when pooled. Here, we mixed equal parts of either B2M-specific XT guide-Cas9-RNP with a non-targeting guide (NTC crXT)-Cas9-RNP. Addition of NTC guide RNAs did not impact efficiency of gene editing in any crRNA format (FIG. 2C, FIG. 7C). These results demonstrate that total RNP concentration is not a significant limiting factor in these assays, and that comparative assessment of individual guide RNAs can reveal highly potent candidates that function efficiently in isolation.

### Example 4: Fully Synthetic sgRNAs Produce Optimal Gene Disruption in Primary Myeloid Cells

Two-part guide RNAs provide a cost-effective and easy-to-produce solution for gene editing purposes. More recently, fully-synthetic single guide RNAs (sgRNAs) have been developed; these link both the crRNA and tracrRNA into a single unit (Jinek et al., 2012). Synthetic sgRNAs allow for chemical modification to increase function and/or stability, and bypass the need for guide annealing prior to RNP complexing (Hendel et al., 2015; Kim et al., 2018; Ryan et al., 2018; Wienert et al., 2018). In accordance with their improved stability or function relative to two-part guides, we observed slight but overall enhanced KO efficiency in both monocyte derived macrophages and dendritic cells with B2M guides 1 and 2 formatted as sgRNAs (FIG. 2D). This improvement was consistent across multiple donors (FIG. 7D). Consistent with our findings using crRNA formats, we observed that Cas9 protein amount did not influence efficiency of gene knockout, nor did the presence of a non-targeting (NTC) sgRNA when combined with B2M-specific sgRNAs (FIG. 7E). Pooling two sgRNAs targeting the same gene modestly improved knockout efficiency, as observed with crRNA formats (FIG. 7E).

It was reasoned that the increased activity of sgRNAs might allow them to be used at a lower gRNA:Cas9 ratio compared to the 3:1 ratio we found to be optimal for crRNAs and crRNAXTs. In effect, this would aid in lowering costs associated with the use of synthetic sgRNAs. We therefore tested a 2:1 sgRNA:Cas9 ratio (B2M sgRNA2:IDTV3 Cas9) and found that this resulted in comparable KO efficiency relative to a 3:1 ratio (FIG. 7F). Encouraged by the maintenance of activity with reduced guide concentration, we evaluated the minimum amount of sgRNA:Cas9 RNP required for effective target disruption. Here, we performed a titration experiment comparing a less active (sgRNA 2) versus a more active (sgRNA 4) sgRNA targeting B2M, with decreasing amounts of RNP in 2-fold increments. As shown for the less active guide (B2M sgRNA 2), KO efficiency dropped by 36.5% when the RNP volume was halved from 4 µL (180 pmol gRNA, 60 pmol Cas9) to 2 µL (90 pmol gRNA, 30 pmol Cas9) (FIG. 2E). We also performed the same titration curve in the presence of “electroporation enhancer” (a single stranded DNA carrier, IDT, 4 µM) and found that, although this had no discernable effect on KO efficiency at 4 µL, the enhancer allowed for a reduction in the sgRNA2-RNP amount to 2 µL without substantial loss in KO efficiency (4 µl: 96.2%, 2µl: 92.9%). Importantly, the enhancer had no effect on cell viability (FIG. 7G, FIG. 7H). To our surprise, the activity curve for the more effective sgRNA displayed a significant shift, with the RNPs retaining near complete activity at the 0.125 µl dose in the presence of the enhancer, or ~16 fold less protein versus that needed for equivalent efficiency with the less potent guide RNA (FIG. 2F). Taken together, we conclude that a 2:1 sgRNA:Cas9 molar ratio in the presence of 4 µM enhancer is optimal, but the minimal amount of RNP to achieve maximal KO efficiency should be tested for each guide as substantial reductions in the effective RNP quantities may be achievable.

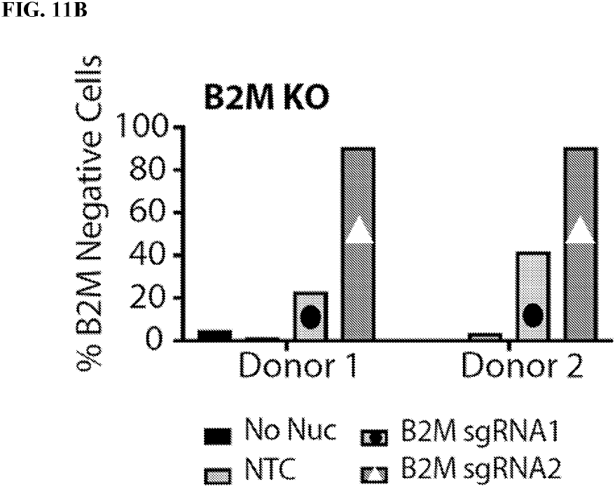

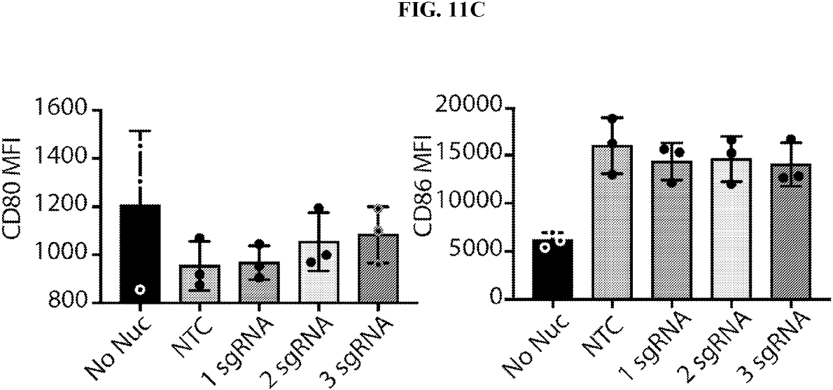

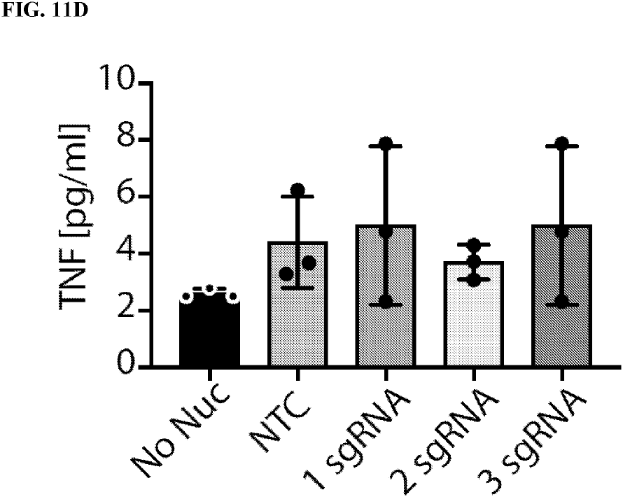

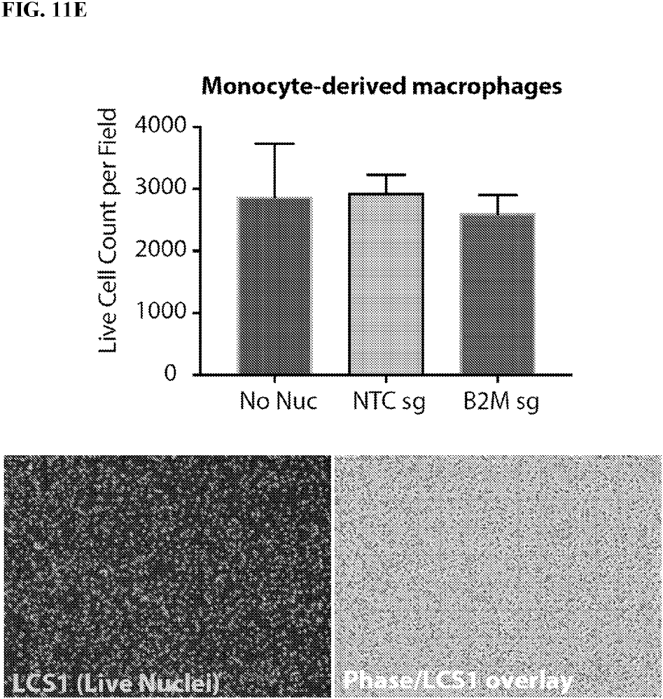

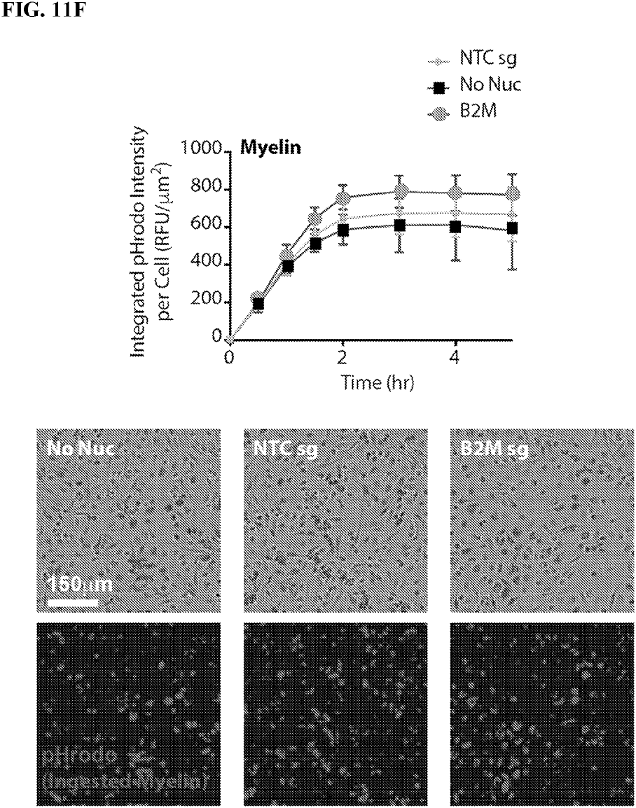

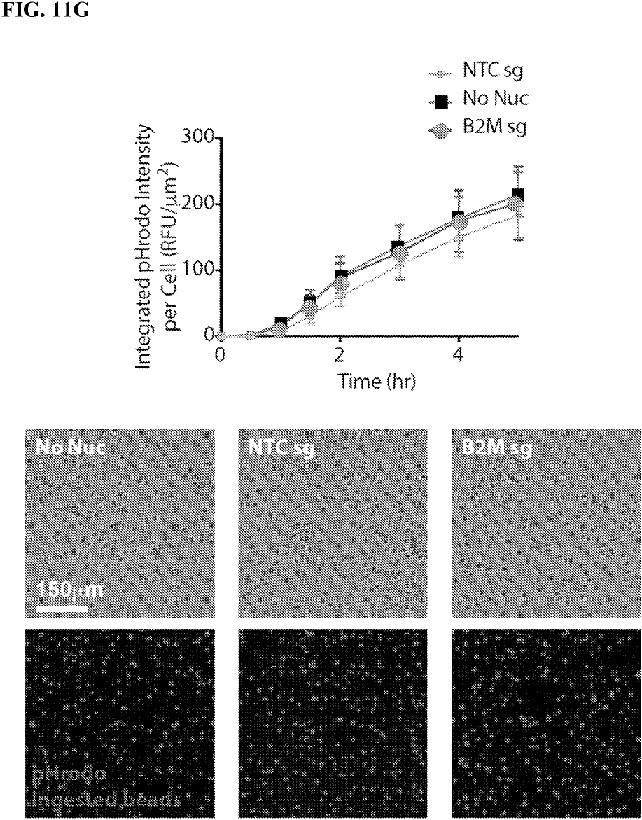

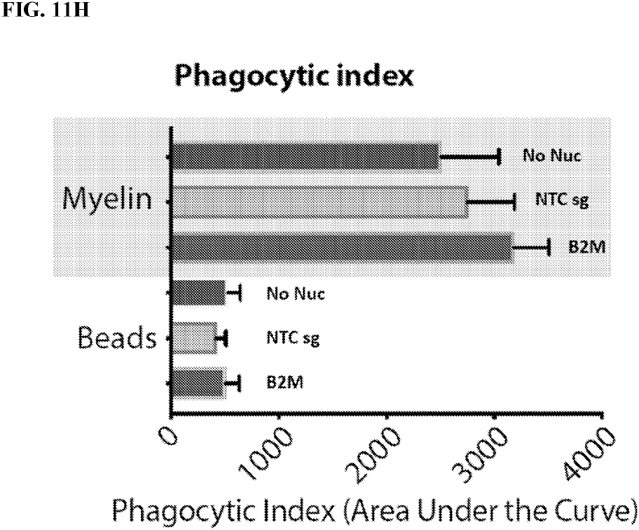

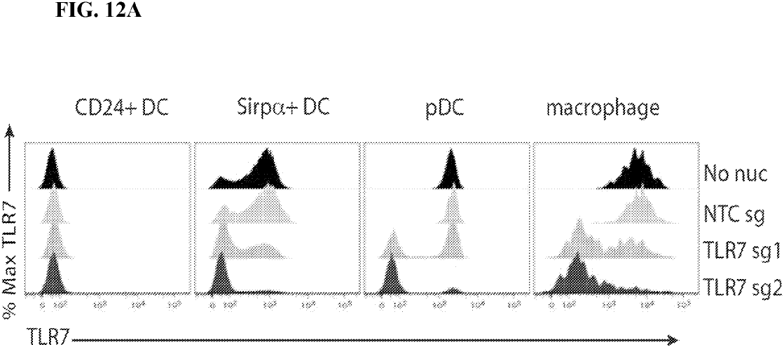

The impact of nucleofection on innate cell activation and cytokine production was assessed. Measuring myeloid cell phenotypic markers on monocyte-derived macrophages from two independent donors revealed comparable cell surface levels of CD14, DC-SIGN, HLA-DR, CD69, CD11c across all experimental conditions (FIG. 11A). CD11b expression was elevated following nucleofection (FIG. 11A). Importantly, the efficiency of gene deletion was not correlated with expression level of any of the tested phenotypic markers (FIG. 11B). We also compared the impact of single or pooled sgRNAs on expression of co-stimulatory proteins and cytokines in monocyte-derived macrophages. Elevated levels of the co-stimulatory protein CD86 but not CD80 were observed upon nucleofection; these were independent of the quantity of gRNA (FIG. 11C). Levels of secreted TNF remained low following nucleofection (FIG. 11D), while Type I interferon (IFNβ) levels were undetectable in all conditions (data not shown).

Finally, the impact of nucleofection on phagocytic capacity of monocyte-derived macrophages was characterized using live-cell imaging. Macrophages were co-incubated with particulate cargo of varying size (myelin debris) or a defined diameter (beads) labeled with a pH-sensitive fluorescent dye (pHrodo-red) and imaged periodically over 5 hours. This permits quantification of cargo uptake as well as delivery to the lysosomal (degradative) compartment of macrophages. Given that cell density has a significant impact on these measurements, live cell counts were determined immediately post-nucleofection to ensure equal cell numbers in each experimental condition. Live cells counts at the end of the assay confirmed equivalent cell densities among non-nucleofected (No Nuc), non-targeted control (NTC sg) and B2M-KO (B2M sg) monocyte-derived macrophages (FIG. 11E). We observed equal rates of phagocytosis of myelin (FIG. 11F) as well as beads (FIG. 11G). We generated a phagocytic index from the time-course data (AUC over 5 hours), which indicated comparable levels of phagocytosis by monocyte-derived macrophages in each treatment group (FIG. 11H). We conclude that nucleofection results in a measurable difference for specific phenotypic and activation markers, but does not broadly induce a phenotypic change in monocyte-derived macrophages. These observations also provide impetus for researchers to use non-nucleofected as well as non-targeting gRNAs as standardized controls in their experimental design.

To determine whether there was additivity between sgRNA-loaded RNPs directed against the same target, similar to two-part guides, the B2M gRNA1 and gRNA2 sequences in the sgRNA format as compared to either gRNA individually were tested (FIG. 7E). As with two-part guides, the pool of sgRNAs resulted in a higher KO efficiency compared to either sgRNA alone (at the equivalent concentration). Also, similar to the crRNAXT condition, the combination of an NTC sgRNA RNP with a B2M sgRNA RNP (complexed separately) reduced the KO efficiency of the B2M RNP, revealing guide competition to be a generalizable feature of Cas9 RNP use (FIG. 7E).

### Example 5: Efficient CRISPR/Cas9 Deletion of Toll-like Receptor 7 in Murine BMDCs

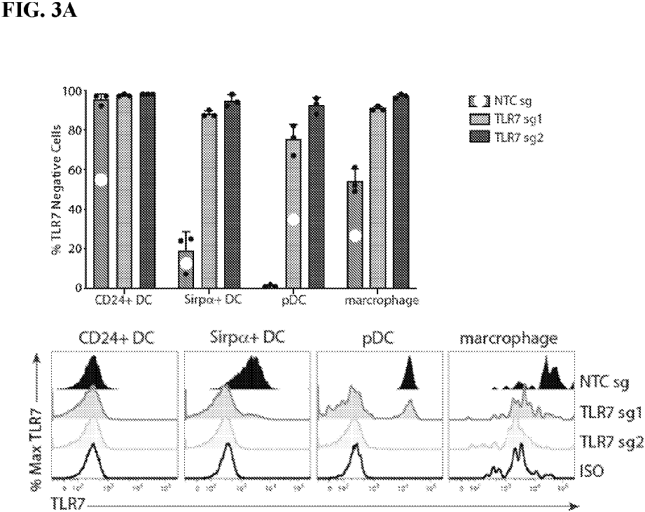

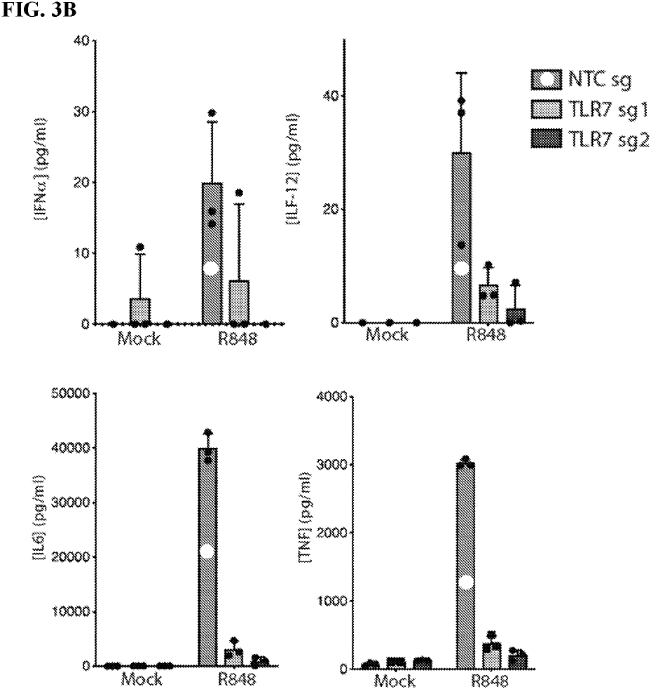

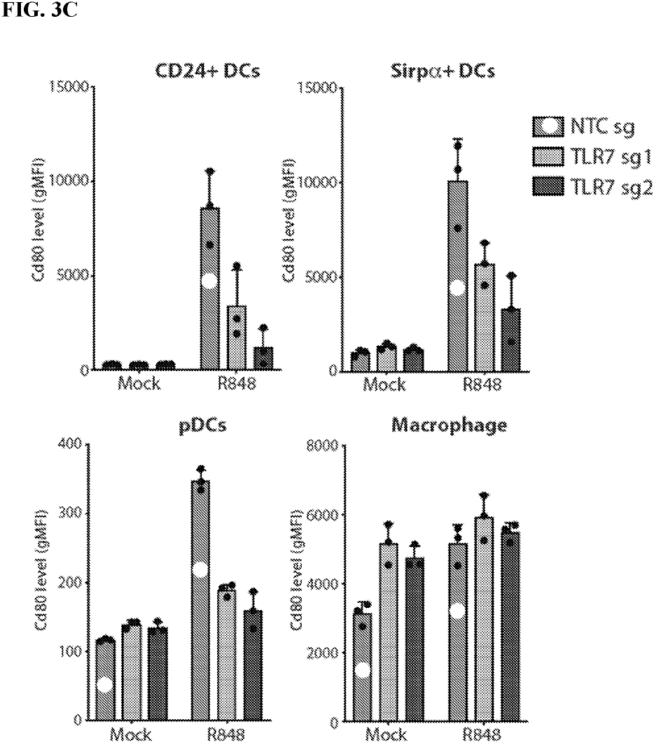

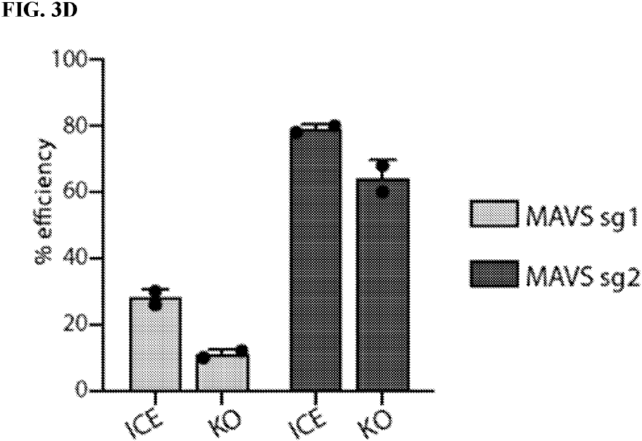

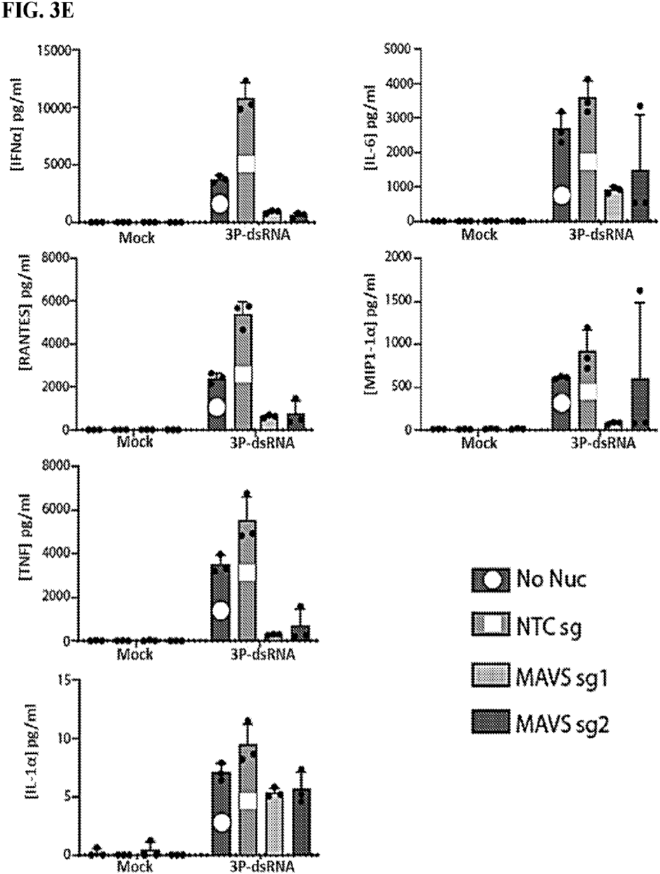

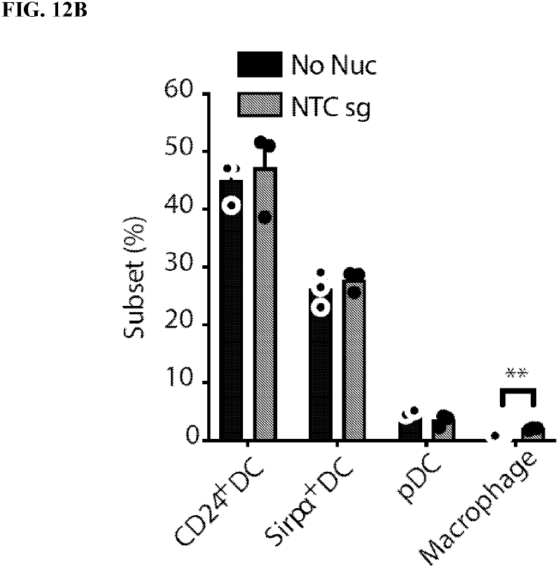

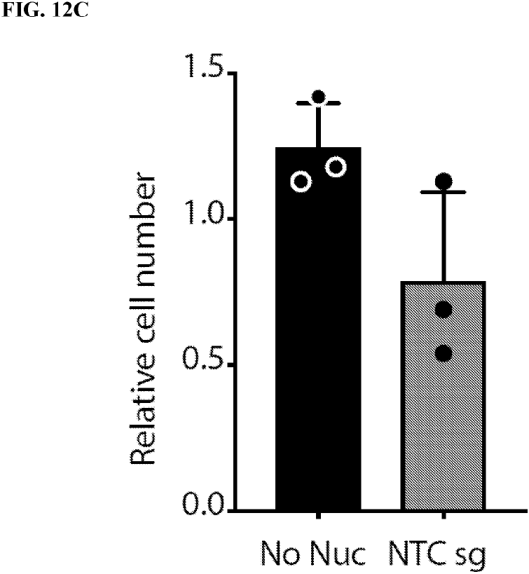

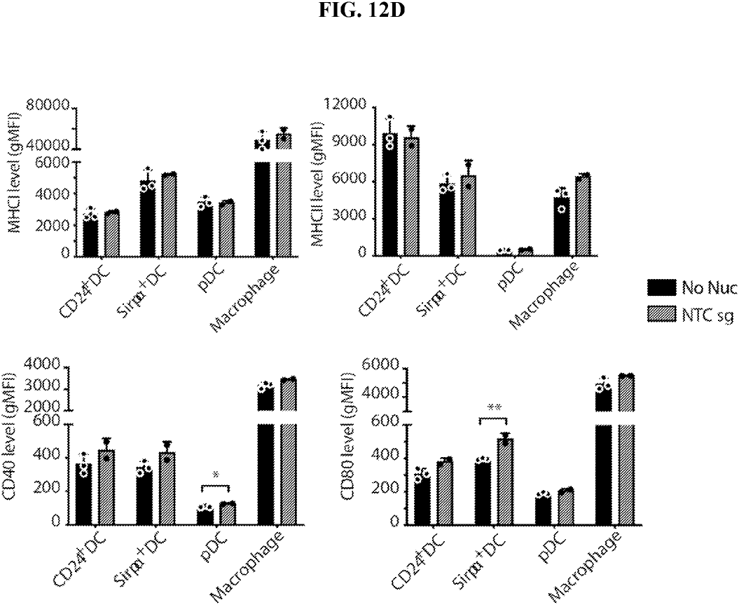

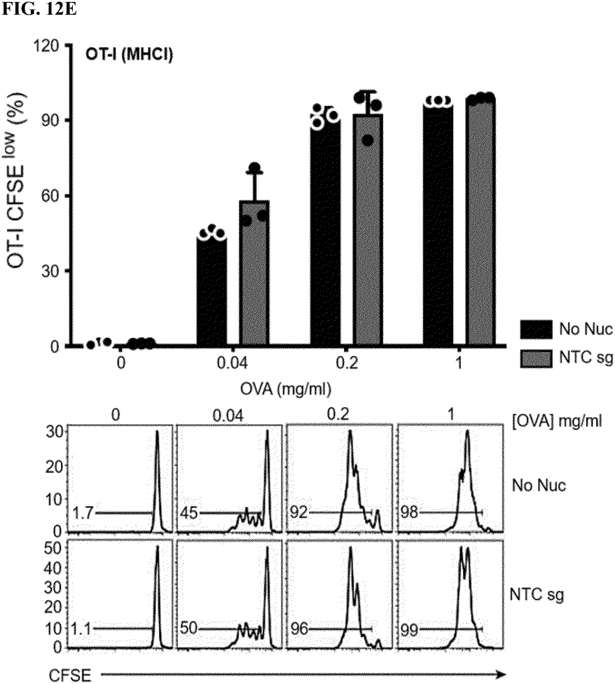

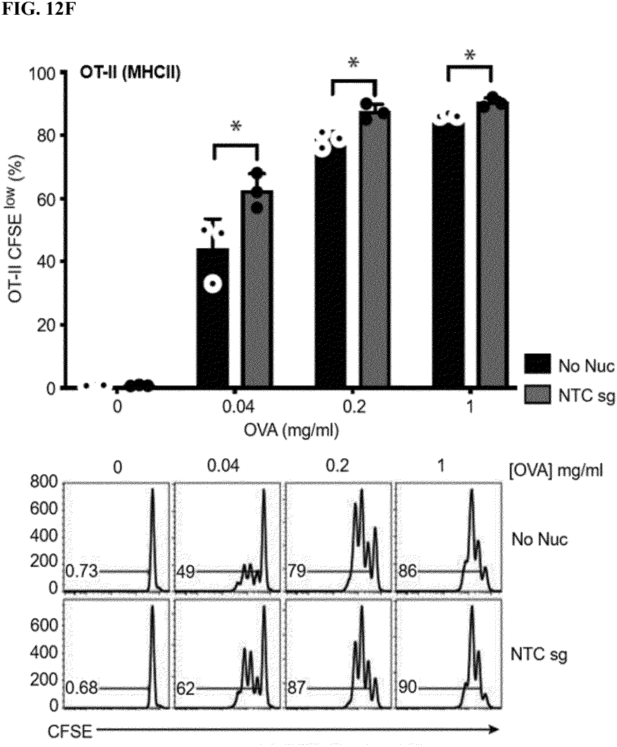

Having determined the optimal conditions for genetic manipulation of primary myeloid cell types, it was next determined whether these protocols could be used to study inflammatory responses. The general enhancements we observed with sgRNAs relative to crRNA variants prompted us to compare sgRNA gene-editing efficiency between the two optimal Cas9-RNP delivery conditions we had previously identified for Flt3 ligand-cultured BMDCs (buffer P3, program CM-137 vs. buffer P3, program EN-138 as in FIG. 1E). We focused on Toll-like receptor 7 (TLR7), a microbe-associated pattern recognition receptor that is highly expressed on pDCs. Two TLR7-specific sgRNAs of different efficiencies were nucleofected into mouse bone marrow cells; these were differentiated for 12 days with Flt3 ligand. Using buffer P3, Program CM-137, we observed that individual sgRNAs led to efficient reduction of TLR7 in all myeloid cell subsets in a manner dependent on the inherent efficiency of each sgRNA (FIG. 3A, CM-137; histograms in FIG. 12A) with the exception of CD24+ DCs, which lacked detectable TLR7 even before nucleofection, as previously shown (Naik et al., 2005). In contrast, Program EN-138 demonstrated decreased efficiency of TLR7 deletion in pDCs and macrophages (FIG. 3A, EN-138). To quantify a cellular response to TLR7 activation, we determined the levels of multiple cytokines in the supernatant. Tlr7 KO reduced levels of IFNα, IL-12p40, TNF and IL-6 in the supernatant in response to stimulation with the TLR7 agonist R848, as compared to the non-nucleofected and non-target control nucleofected samples (FIG. 3B). This demonstrates that the protocol we have developed is able to generate efficient KO of Tlr7 and abolish downstream cytokine responses in BMDCs while maintaining normal cell physiology.

To broadly assess the impact of sgRNA nucleofection on cell differentiation and function, myeloid cell phenotypes, activation and function were compared following nucleofection with non-targeted sgRNA (NTC). While relative abundances of DC subsets were not impacted by nucleofection, we noted a modest (~5%) increase in macrophage abundance when compared to non-nucleofected cells (FIG. 12B). A decrease in overall cellular yield was noted following nucleofection, likely an immediate consequence of nucleofection on the total bone marrow at day 0 (FIG. 12C). Comparing expression of activation and maturation markers revealed comparable levels of MHCI and MHCII across all cell subsets but elevated levels of CD40 on pDCs and CD80 on SIRPα+ DCs (FIG. 12D). Finally, we compared MHCI and MHCII-dependent antigen presentation. Following nucleofection and differentiation as above, BMDCs were pulsed with varying concentrations of chicken ovalbumin (OVA) and co-cultured with antigen specific CD8+ T cells (OT-I) or CD4+ T cells (OT-II). T cell proliferation was measured using flow cytometry after 3 days. OT-I proliferation was comparable between non-nucleofected and nucleofected conditions (FIG. 12E). Nucleofection enhanced OT-II proliferation at all concentrations of OVA (FIG. 12F). These changes, while mostly modest, require consideration when investigating specific myeloid cell subsets purified from the mixed culture system of Flt3 ligand-driven differentiation. Cumulatively, we recommend using Buffer P3 and Program CM-137 for optimal gene editing when using single guide RNA (sgRNA) chemistry.

### Example 6: Efficient Single and Multiplexed Deletion of Ticam1/TRIF and MyD88 In Murine Bone Marrow-Derived Macrophages

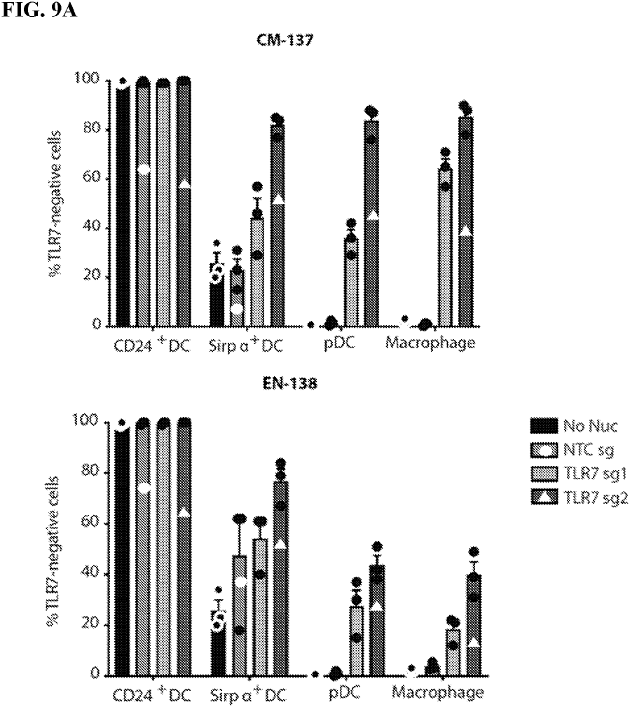

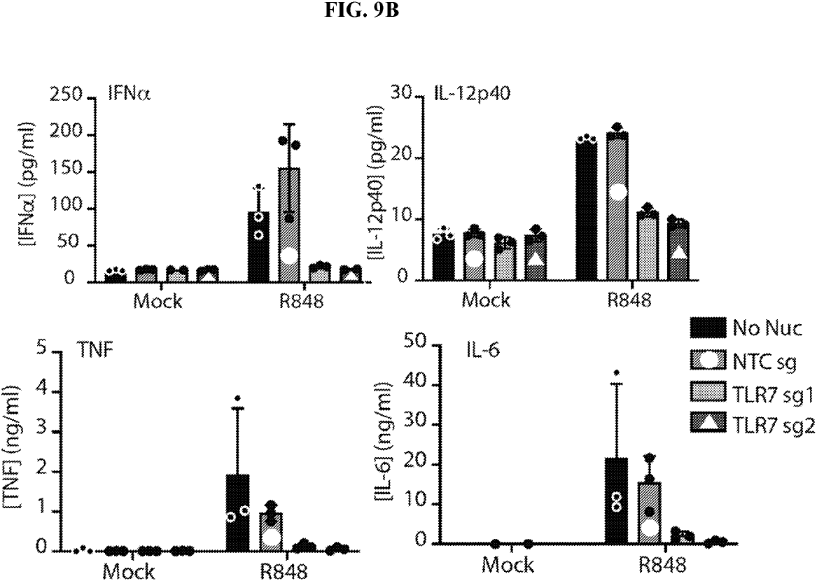

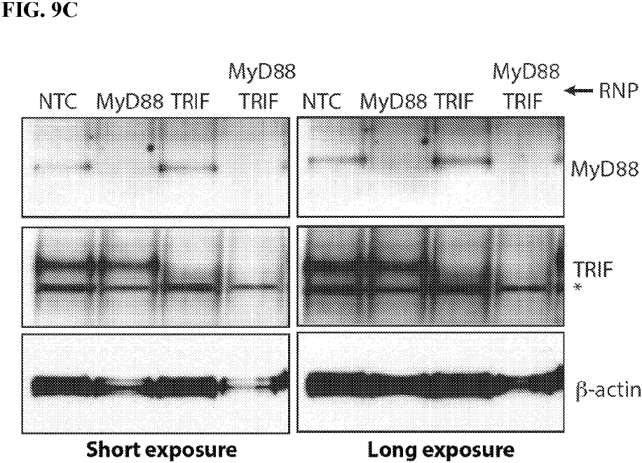

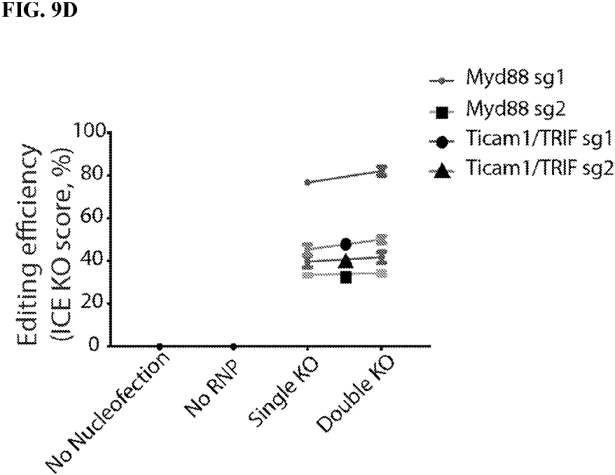

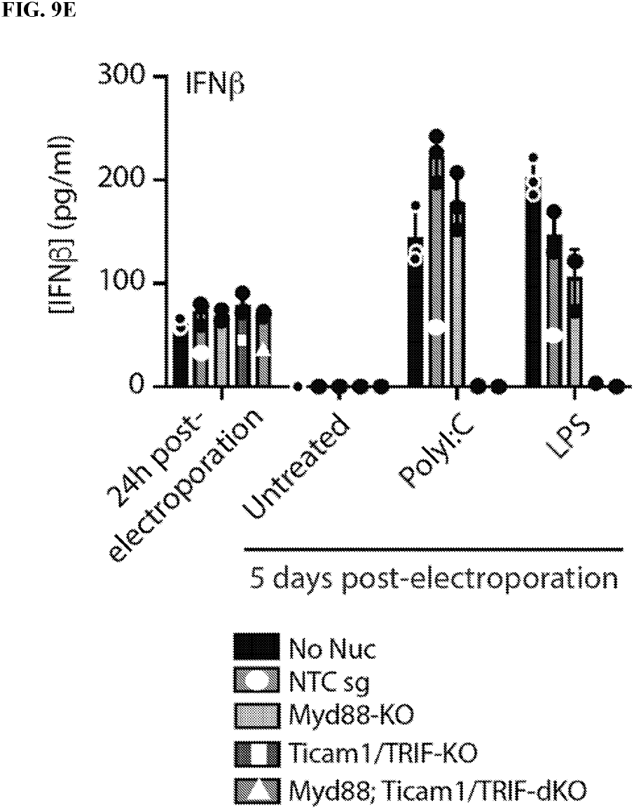

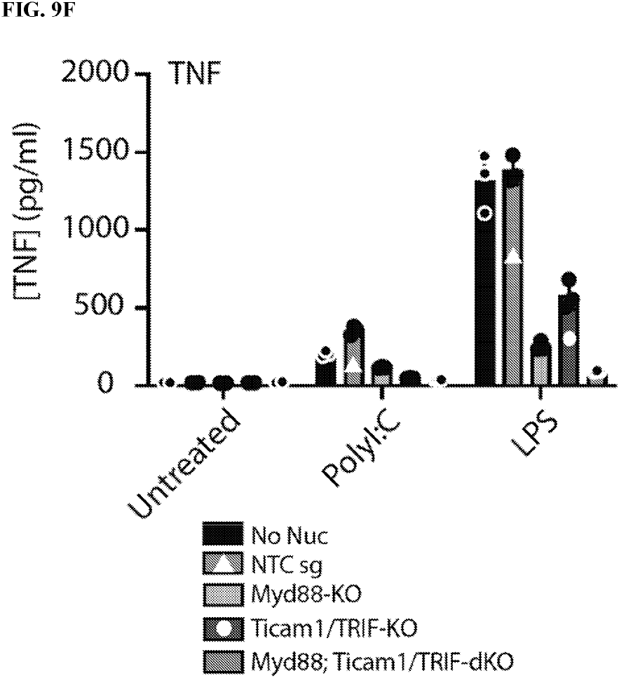

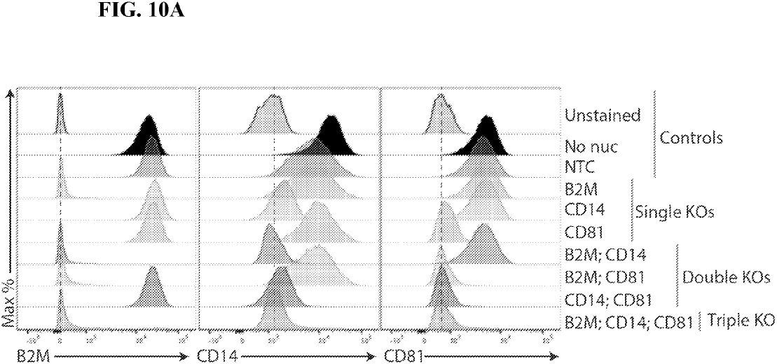

Next, the ability of this protocol to generate single or multiple gene knockouts in BMDMs was tested in the context of TLR signaling. Macrophages use cytosolic adaptors MYD88 or TRIF (encoded by Ticaml) to engage Toll-like receptors (TLRs) for anti-microbial immunity. While the bacterial cell wall antigen LPS activates TLR4 to signal via both adaptors, double-stranded RNA engages TLR3 and only signals via TRIF (Gay et al., 2014). Two sgRNAs per target gene were pooled to generate single or double knockouts (FIG. 9C). Sanger sequencing analysis of individual guide RNAs revealed that Myd88 sgRNA1 exhibited the highest editing efficiency, whereas both Ticam1/TRIF sgRNAs exhibited ~50% editing efficiency. These were unchanged in single or double knockout conditions (FIG. 9D). Measurement of IFNβ secretion by BMDMs was comparable across all conditions 24 hours post-nucleofection, demonstrating lack of an enhanced interferon response, and thus generalized activation, following Cas9-RNP delivery (FIG. 9E). Engaging TLR3 with PolyI:C (a synthetic double-stranded RNA analog) induced IFNβ and TNF by control (NTC) and Myd88-KO but not by those lacking Ticam1/TRIF (i.e. Ticam1/TRIF-KO or Myd88;Ticam1/TRIF-dKO) (FIGS. 9E, F, PolyI:C treatment). Similarly, engaging TLR4 with LPS revealed that Ticam1/TRIF mutation was sufficient to abolish IFNβ secretion (FIG. 9E, LPS treatment), whereas TNF secretion was dependent on a combination of MYD88 or TRIF signaling (FIG. 9F, LPS treatment). Together, our results confirm the successful generation of compound knockout BMDMs in a physiologically-relevant context of innate anti-microbial immunity.

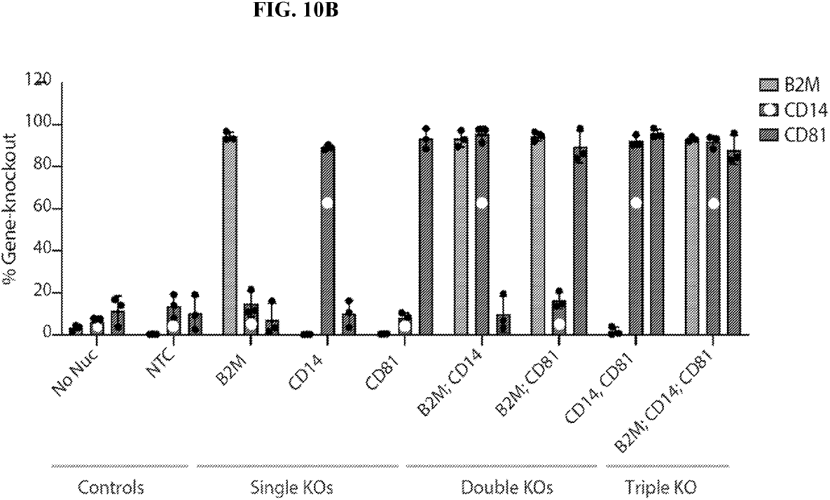

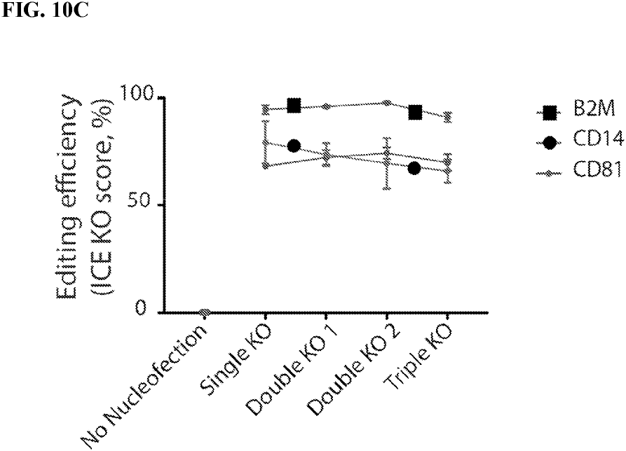

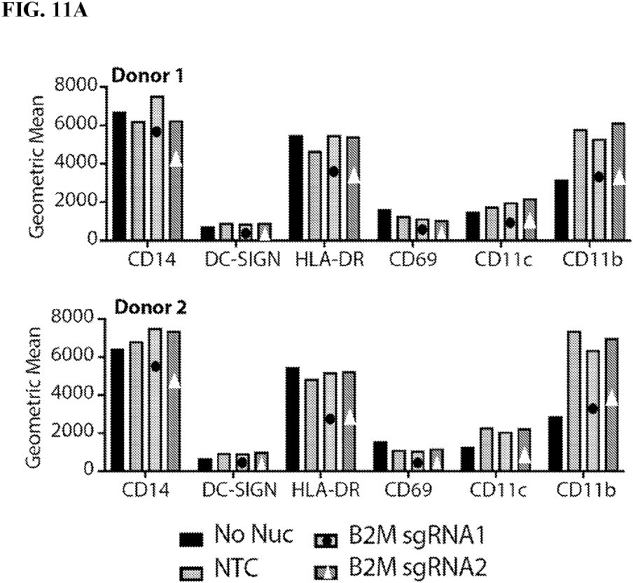

Finally, generation of single and multiplexed gene knockouts in monocyte-derived macrophages from human donors was demonstrated. We chose to delete B2M along with CD14 and CD81 which would enable quantitative flow cytometry-based assessment of gene editing efficiency. A single sgRNA targeting each gene was introduced into monocytes by nucleofection using the Buffer P3, CM-137 protocol; cells were then differentiated into macrophages using M-CSF. On day 6 post-nucleofection, cells were harvested to compare gene knockouts. Flow cytometry revealed near complete, population-wide editing in single, double or triple knockout samples (FIG. 10A, representative histograms; FIG. 10B, quantification of gene knockout). Editing efficiency was confirmed by ICE analysis (Synthego), which provides a measure of mutant allele frequency across sequenced PCR products derived from the targeted loci (FIG. 10C). Therefore, high-functioning, individual gene-specific sgRNAs can be combined for one-step production of multiplex, population-wide KOs in primary human myeloid cell types without the need for selection or stable gene editing component expression.

### Discussion

This study provides a rapid, efficient and economical method to generate population-level gene knockouts in primary myeloid cells of human and murine origins (summarized in Tables 1 and 2). Optimizations developed herein permit single and multiplexed gene knockouts (up to 3 genes at a time) with >90% efficiency, thus eliminating a need for step-wise gene disruption and/or transgenic marker selection. Results reveal that combining pairs of gene-specific crRNAs provides an additive, optimal effect on knockout efficiency. Alternatively, individual chemically-synthesized sgRNAs can be mixed and employed for near-complete, compound gene disruption. This study also demonstrates that the addition of the IDT electroporation enhancer in the context of human monocyte-derived cells can both considerably increase the gene editing efficiency of suboptimal gRNA sequences and reduce the effective dose of active sgRNAs. By increasing the repertoire of functional guide RNA sequences while at the same time reducing the anticipated costs and material demands associated with generating a KO cell type, we have ameliorated separate barriers for high-throughput KO analysis in primary human and mouse myeloid cells. Given the observation that human monocytes upregulated activation markers following nucleofection, it is prudent to monitor their activation states and use non-electroporated cells as controls when investigating innate inflammatory phenotypes. Comparison of additional markers revealed a lack of elevation following nucleofection, demonstrating that this protocol has limited, if any, effect on human myeloid cell activation status.

The comparison of guide RNA chemistries in BMDCs revealed pDCs to be particularly impacted by crRNA vs. sgRNA, where CRISPR-KO was achieved as efficiently in pDCs as in other cell types of BMDC culture with sgRNAs versus crRNAs. Since pDCs predominantly differentiate from common lymphoid progenitors (CLPs) — a lineage distinct from myeloid lineage progenitors (Rodrigues et al., 2019) — CLPs may be targeted more efficiently by sgRNAs. This observation warrants further assessment of CRISPR-Cas9 RNP-mediated gene editing in hematopoietic progenitors. It is also important to note that myeloid cells edited using our methods were cultured using varying cell adhesion conditions. For instance, murine monocyte-derived macrophages and BMDMs were maintained in low attachment tissue culture (TC) multi-well plates or non-TC treated Petri dishes, respectively. BMDCs were differentiated in conventional TC-treated multi-well plates. For assessment of gene knockout and functional assays, BMDMs were initially cultured on Petri dishes and then transferred to TC-treated multi-well plates. Across all adhesion conditions, we observed comparable efficiency of gene knockout. Thus, myeloid cell adhesion does not materially impact efficiency of gene editing or viability of cells following nucleofection with the optimized protocols generated herein.

We recently described the utility of this method in studying necroptosis, where Cas9-RNP delivery into murine BMDMs was used to dissect signaling nodes of TRIF-mediated cell death (Lim et al., 2019). Beyond this example of reverse genetics, the scalability our findings is likely to enable functional screening in primary myeloid cells. This is particularly relevant to human immunology given the known, but as yet broadly unexplored, phenotypic differences between human and murine immune cells. With our approach, arrayed platforms of focused cr/sgRNA libraries can be utilized to reveal phenotypes of interest in donor-derived human myeloid cells followed by assessment in pre-clinical model systems such as mice. Furthermore, the ability to deliver significant quantities of recombinant Cas9 permits pooled functional screening in myeloid cells derived from knockout or mutant mouse strains. This alleviates the need to breed a genotype of interest with a Cas9-knock-in strain, thus providing significant advantages in study design and economy. Finally, the lack of significant chronic immune cell activation observed when employing our methods generate the possibility of in vivo evaluation via adoptive cell therapy following RNP-mediated gene knockout.

These findings provide a significant technical advance in the study of myeloid cells, typically considered a challenging cell type for gene editing. Looking ahead, adapting our methods for alternative gene regulation (e.g. via CRISPRi or CRISPRa) or SNP/reporter knockin strategies will further expand our ability to investigate and modify innate immunity in the relevant primary cell subsets(s) of interest.

TABLE 1

Optimal conditions for Cas9/RNP-mediated gene deletion in murine myeloid cells

| | | | | | | |
|---|---|---|---|---|---|---|
| | Monocyte | Macrophage (MCSF) | CD24+DC (FLT3L) | Sirpα+DC (FLT3L) | pDC (FLT3L) | Macrophage (FLT3L) |
| Program | CM-137 | CM-137 | CM-137 | CM-137 | EN-138 (crRNA, crRNAXT); CM-137 (sgRNA | CM-137 |
| Buffer | P3 | P3 | P3 | P3 | P3 | P3 |
| Cell number ranger per reaction in 20 µL volume | 0.5-5 × 10⁶ | 0.5-5 × 10⁶ | 2-6 × 10⁶ total bone marrow cells | 2-6 × 10⁶ total bone marrow cells | 2-6 × 10⁶ total bone marrow cells | 2-6 × 10⁶ total bone marrow cells |
| Culture conditions following electroporation | BMDM culture media containing MCSF | BMDM culture media containing MCSF | BMDC culture media containing FLT3L | BMDC culture media containing FLT3L | BMDC culture media containing FLT3L | BMDC culture media containing FLT3L |

TABLE 2

Optimal conditions for Cas9/RNP-mediated gene deletion in human myeloid cells

| | | |
|---|---|---|
| | Monocyte derived macrophage (M-CSF) | Monocyte derived DC (GM-CSF+IL-4) |
| Program | CM-137 | CM-137 |
| Buffer | P3 | P3 |
| Cell number ranger per reaction in 20 µL volume | 0.5-5 × 10⁶ fresh or thawed monocytes | 0.5-5 × 10⁶ fresh or thawed monocytes |
| Culture conditions following electroporation | Mo-Mac culture media containing M-CSF | Mo-DC culture media containing M-CSF + IL-4 |

### Example 7: Materials and Methods

### Mice

All mice in this study were used following Genentech Institutional Animal Care and use Committee (IACUC). All experiments were conducted following protocols approved by IACUC. Female eGFP-transgenic mice were obtained from Jackson Laboratories (C57BL/6-Tg(CAG-EGFP)1Osb/J, stock no. 003291). Female wild-type C57BL/6J mice were obtained from Jackson Laboratories (stock no. 000664). All mice were aged 8-12 weeks.

### Human Donors

Peripheral blood and PBMCs were collected from healthy donors participating in the Genentech blood donor program using written, informed consent from the Western Institutional Review Board.

### Human Monocyte Preps

PBMCs were isolated using Sepmate tubes (StemCell Technologies cat# 84540) and ACK Lysing Buffer (Fisher cat# A1049201) following manufacturer’s protocol from buffy coats or leukopaks from healthy donors. Human monocytes were isolated from the PBMCs using the Human monocyte isolation kit (Miltenyi # 130-091-153) according to the manufactures protocol. Monocytes were aliquoted and frozen in 10% DMSO and FBS for further use.

### Monocyte Derived Dendritic and Macrophage Cell Cultures

Monocyte derived dendritic cells were cultured in DC medium (RPMI supplemented with 10% FBS (Gibco), 2 mM 1-alanyl-1-glutamine (GlutaMAX; Gibco), 55 µM β-mercaptoethanol (Gibco), 100 U/ml penicillin, 100 µg/ml streptomycin (Thermo Fisher 15140122), and cytokines GM-CSF 800 U/ml (Peprotech) and IL-4 500 U/ml (Peprotech) at a density of 1×10⁶ cells per ml. Medium was changed every 2-3 days by removing ½ the volume of medium in the well, spinning down harvested cells at 400 xg for 5 min, and then resuspending spun cells in DC medium with 2X cytokines and replating with remaining cells. Monocyte derived macrophages were cultured in Mac medium (DMEM High glucose supplemented with 10% FBS (Gibco cat# 10082-147), 2 mM 1-alanyl-1-glutamine (GlutaMAX; Gibco), 100 U/ml penicillin, 100 µg/ml streptomycin (Thermo Fisher cat# 15140122) and M-CSF 100 ng/ml (Peprotech). Medium was changed every 2-3 days by adding ½ volume of medium with 1X cytokines into each well.

### Optimized Human Monocyte Dendritic and Macrophage Cell KO

gRNA Selection. All gRNA sequences were chosen using the IDT predesigned guides searchable on their website, or an in-house algorithm.

Preparation of cells. Cells were isolated as described above. Frozen monocytes were thawed into Not Treated 6-well plates (Fisher cat# CKS336) at 1e6 cells/ml in DC or Mac medium with appropriate cytokines and cultured overnight.

Preparation of gRNAs. To prepare each gRNA, the Alt-R crRNA or crRNAXT (crRNA(XT)), Alt-sgRNA and Alt-tracrRNA (cat# 1072534; IDT) were reconstituted to 100 µM with Nuclease-Free Duplex Buffer (IDT). To prepare the crRNA(XT)-tracrRNA duplexes, the two oligos were mixed at equimolar concentrations in a sterile PCR tube (e.g., 5 µl Alt-R crRNA, crRNA(XT) with 5 µl Alt-R tracrRNA). Oligos were annealed by heating at 95° C. for 5 min and the mix was cooled to room temperature and left to hybridize for 15 min at room temperature in PCR thermocycler (program: 95° C. for 30 sec, 95° C. for 4.5 min, 25° C. for infinite). The mix was then placed on ice or frozen at -20° C. until further use.

Precomplexing of Cas9-RNPs. In a sterile PCR strip or 1.5 ml tube the annealed crRNA(XT)-tracrRNA duplexes or sgRNAs were mixed with Cas9 (IDT SpCas9 “Alt-R® S.p. Cas9 Nuclease V3″, or Thermo SpyCas9 “TrueCut Cas9 Protein V2”) at a 3:1 molar ratio for crRNA(XT)s (3 µl of crRNA(XT)-tracrRNA duplex + 2 µl Cas9 5 mg/ml) or for sgRNAs at a molar ratio of 2:1 (2 µl sgRNA + 2 µl Cas9 5 mg/ml) for each reaction unless otherwise indicated. The Cas9-RNPs were then incubated at room temperature for at least 20 min.

Nucleofection of Cas9-RNP complexes. Appropriate medium (DC or Mac) was pre-warmed in a cell culture plate at 37° C. in an incubator for at least 30 min. Cells were harvested for nucleofection. For monocyte derived DCs, the cells were collected from the 6-well plate and spun at 400 xg for 5 min. Then >300 ul of 1X PBS was added to the each well, collected and spun at 400 xg for 5 min to harvest any remaining cells. The monocyte derived macrophages were harvested similarly, with the exception that Detachin (Genlantis cat #T100100) was added after the PBS harvest and the 6-well plates were incubated for 5 min at 37° C. to allow any attached cells to be released and collected. For both DCs and Macs, the final cell pellets were then washed twice with >5ml of 1X PBS and counted. 1×10⁶ cells per reaction were resuspended in 20 µl of P3 primary nucleofection solution (P3 Primary Cell 4D-Nucleofector X Kit, cat# V4XP-3032). The 20 µl of cell/P3 nucleofection solution mix was then added to each Cas9-RNP complex and pipetted up and down 3-5 times gently to mix while avoiding bubbles. The cell-RNP mix was then immediately loaded into the supplied nucleofector cassette strip. The strip was inserted into the Lonza 4D-Nucleofector (4D-Nucleofector Core Unit: Lonza, AAF-1002B; 4D-Nucleofector X Unit: AAF-1002X) and electroporated with the Buffer P3, CM-137 condition. The cassette strip was removed and 150-180 µl of prewarmed medium was immediately added into each cassette well. The medium/cell-RNP mix was then pipetted into the appropriate cell culture dish and the cells were cultured as described above for five (macrophage) or seven (dendritic cells) days. The KO efficiency was assayed by FACS.

Enhancer and Cas9-RNP titration analysis. Monocyte derived macrophage cells were harvested and electroporated with sgRNA-containing-Cas9-RNP complexes using the P3, CM-137 condition as described above with the following modifications. The amount of Cas9-RNP complex was titrated down from 4 µl (2 µl sgRNA + 2 µl Cas9 at 5 mg/ml) to 0.0019 µl with 2-fold dilutions. In the conditions including an electroporation enhancer, the same titration was performed with the addition of 1 µl (4 µM) of IDT enhancer (IDT cat# 1075915) to the Cas9-RNP complex prior to electroporation during complexing.

### FACS Analysis for Human Monocyte Dendritic Cells and Macrophages

Cells were harvested as described above for nucleofection, then stained with LIVE/DEAD Fixable Aqua Dead Cell dye (ThermoFisher cat# L34957) in 50 µl of 1X PBS for 10 min at room temperature. Cells were washed by the addition of 150 µl of 1X PBS to each well and then the cells were centrifuged for 3 min at 1600 rpm. The pelleted cells were resuspended and incubated for at least 30 min with fluorophore-conjugated antibodies in 50 µl of FACS buffer at 4° C. Cells were washed as before and fixed with 2 % PFA for 10 min at room temperature. Cells were then washed with 1X FACS buffer and resuspended in 180 µl of FACS buffer for analysis by flow cytometry. Monocyte derived macrophages and dendritic cells were identified by expression of CD64 or DC-SIGN, respectively. KO was determined by gating for negatively stained cells using the non-targeting control (see FIG. 2A for example gate). Heat-killed cells and unstained cells were used as controls for live-dead and positive antibody staining. All samples were analyzed by the FACSymphony system (BD).

### MAVS KO of Dendritic Cells and Functional Analysis

MAVS KO. Monocyte derived dendritic MAVS KO cells were created as described above using sgRNAs targeting two different sequences. As a control, cells were also electroporated with Cas9-RNPs loaded with a non-targeting control (NTC) sgRNA. The cells were harvested on day 6 after nucleofection and counted. 150 K cells/well for each genotype were plated in triplicate for two conditions (Mock and the RIG-I agonist 5′-3p-dsRNA stimulation (Coch et al., 2013)) in 150 µl of DC medium (no cytokines). The remaining cells for each genotype (>300 K cells per sample) were divided in half to create technical replicates, spun for 5 min at 2,000 rpm and the pellet was washed twice with 1X PBS and then snap-frozen for gDNA preparation and TIDE analysis.

Cell Stimulation. Plated cells were stimulated by the addition of the 5′-3p-dsRNA agonist targeting RIG-I. The 5′-3p-dsRNA was delivered to the cells using Lipofectomine 2000 (Thermo Fisher cat# 11668019) following standard protocols. Briefly, 0.1 µg of 5′-3p-dsRNA or 0.5 µl per well of Lipofectamine 2000 were resuspended in 25 µl per well of OptiMEM and incubated for 5 min at room temperature, then the 5′-3p-dsRNA and lipofectamine suspensions were mixed together and allowed to incubate another 20 min at room temperature. 50 µl of the combined RNA-lipofectamine mix was then added to each well of all three genotypes (NTC, MAVS sg1, MAVS sg2). For the mock control, 50 µl of pre-warmed DC media was added to the cells. The cells were stimulated overnight and then the supernatants were harvested and the cytokines levels (IFNα, IL-6, MIP-1α, TNF, RANTES, IL-1α) were measured by Luminex (Bio-Rad Laboratories). The cells were harvested as described above using Detachin and plated in a U-bottom, TC-treated 96-well plate for intracellular FACS staining to determine MAVS KO efficiency.

Intracellular FACS Staining. FC-block (5 µl/well) (Biolegend cat# 422301) and the live/dead fixable blue stain (0.5 µl/well) (ThermoFisher cat# L34961) was added in 50 µl 1X PBS to all wells (except the unstained controls). The cells were then stained for MAVS using the eBioscience Intracellular Fixation & Permeabilization Buffer Set (ThermoFisher cat# 88-8824-00) following the manufacturer’s protocol. Following staining the cells were resuspended in 180 µl of FACS buffer for analysis. Heat-killed cells and unstained cells were used as controls for live-dead and positive antibody staining. All samples were analyzed by the FACSymphony system (BD).

### PKR KO of Monocyte Derived Macrophages and Functional Analysis

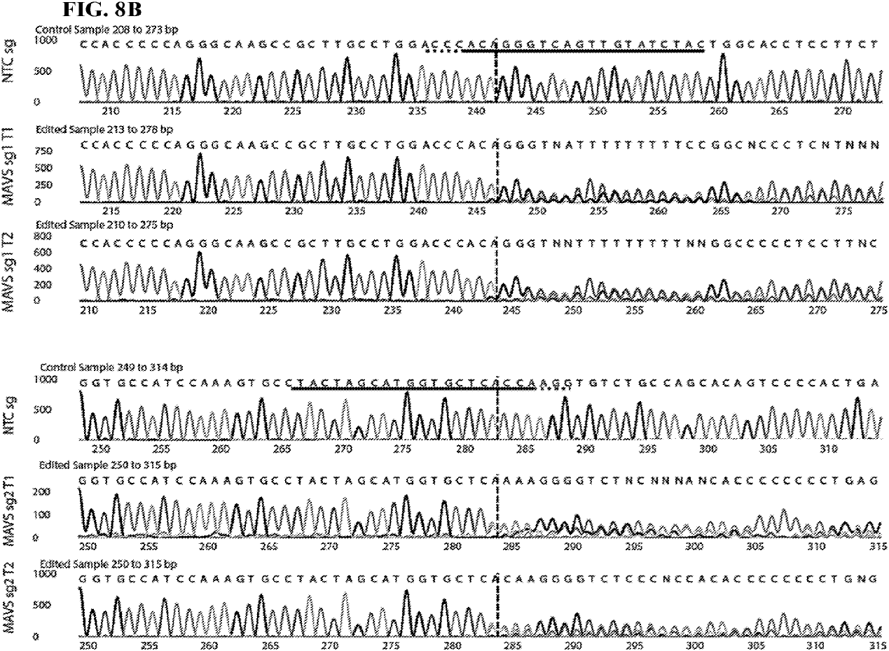

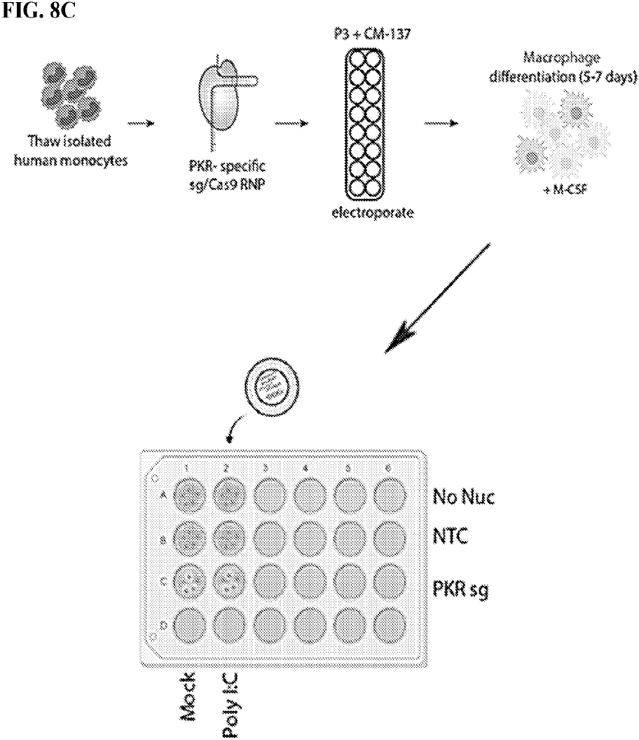

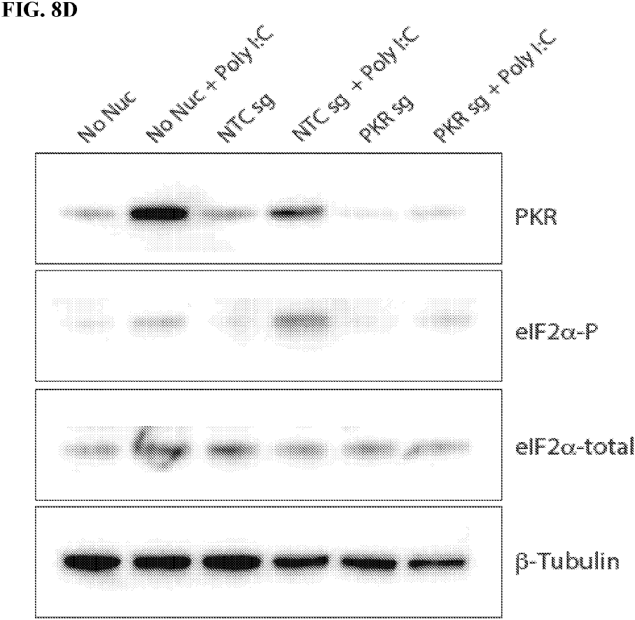

PKR KO and cell stimulation. Monocyte derived macrophage PKR KO cells were created using a single sgRNA (PKR sg) as described above, with the exception that 3×10⁶ cells were electroporated per reaction. As a control, cells were also electroporated with Cas9-RNPs loaded with the NTC sgRNA. Following electroporation, cells for all three conditions (No Nucleofection control, NTC and PKR sg) were plated at 1×10⁶ cells per ml in Mac medium supplemented with cytokines in a TC-treated 24-well plate (plating schematic FIG. 8C). On day 5 after electroporation, one replicate of each cell condition was stimulated with poIy I:C (Invivogen, tlrl-pic). For poly I:C stimulation, 6 ug of poly I:C (2 µg/ml) was added to 500 µl of Opti-MEM. Then 12 µl of TransIT (Mirus, MIR 2225) was added to 500 µl of Opti-MEM and incubated at room temperature for 5 min. The TransIT mix was then added to the poly I:C mix and incubated for an additional 20 min at room temperature. The complexes were then added to the cells.

Western blot analysis. The cells were stimulated for 6 hrs and then harvested as described above using Detachin. The cells were pelleted, washed twice with 1X PBS and the pellet was resuspended in RIPA buffer supplemented with protease inhibitors (cOmplete, Mini, EDTA-free Protease Inhibitor Cocktail Tablets, # 4693159001). Lysates were clarified by spinning for 10 min at 13,000 rpm at 4° C., and the protein content was measured by BCA (Pierce, #23225). For each sample, 10 µl of protein was separated by SDS-PAGE, transferred onto nitrocellulose membrane, and blotted according to standard protocols. Chemiluminescence was imaged using a BioRad ChemiDoc imaging system.

Analysis of CRISPR KO BMDMs for deletion of MYD88, TRIF and STING was performed by lysing cells in RIPA buffer as above. SDS-PAGE was performed using a 4-12% gradient Bis-Tris gel (Novex) followed by protein transfer onto PVDF membranes and standard downstream immunoblotting. Chemiluminescence was detected by enhanced chemiluminescence (Western lightning-plus ECL, Perkin Elmer).

### Murine BMDM Culture

Bone marrow was harvested from tibiae and femurs of mice. Total bone marrow cells were plated in BMDM culture media [DMEM High Glucose, 10% FBS (VWR), GlutaMAX (Gibco) and penicillin/streptomycin (Gibco) supplemented with 50 ng/mL recombinant murine M-CSF (Genentech)] at a density of 0.5×10⁶ cells permL in 150 mm non-TC treated Petri dishes (VWR) in a volume of 20 mL per dish. Two days, 20 mL fresh BMDM culture media was added without removal of media. On day 4, all media was removed and 20 mL fresh BMDM culture media added. On day 5, cells were gently scraped from dishes using a rubber policeman and transferred to a 50 mL conical tube. The Petri dish was washed once with 20 mL 1X PBS and cells harvested via centrifugation. Cells were resuspended in 10 mL 1X PBS and counted, after which they were centrifuged once again for resuspension in appropriate buffers or media for downstream assays.

### BMDM CD11b CRISPR KO Screen

Day 5 BMDMs were resuspended in nucleofection solutions for primary cells (Primary Cell Optimization 96-well Nucleofector Kit, cat# V4SP-9096, Lonza) at a density of 5×10⁵ cells per reaction in 20 µl nucleofection solution and mixed with Cas9-RNP containing Itgam targeting gRNAs. This mixture was electroporated using the Lonza 4D Nucleofector (4D-Nucleofector Core Unit: Lonza, AAF-1002B; 4D-Nucleofector X Unit: AAF-1002X). Immediately following electroporation, ~180 µl pre-warmed BMDM culture media was added to each well and cells harvested by gently washing the well. Each reaction was transferred to a single well in a 6-well TC-treated plate containing 2 mL of pre-warmed BMDM culture media. Cells were cultured for an additional 5 days with complete media changes at 2 and 4 days following electroporation. On day 5, cells were harvested by gently scraping them with a rubber policeman. Harvested cells processed for flow cytometry. First, cells were stained with an Fc-Blocking reagent (CD16/32 FcR block, BD Biosciences) for 10 min at 4C, followed by staining with an antibody cocktail for CD11b (anti-CD11b-APC, CD45 (anti-CD45-FITC). Cells were washed twice in flow cytometry buffer and resuspended in flow cytometry buffer containing a viability marker (Propidium Iodide, PI). Flow cytometry was performed to compare each electroporation condition using a BD Fortessa analyzer. Loss of cell surface CD11b, shown as a drop in its mean fluorescence intensity (MFI) along with maintenance of cell viability, shown as a lack of PI-positive signal, was used to rank each condition.

### Murine Monocyte Culture and eGFP CRISPR KO Screen

Bone marrow was harvested from tibiae and femurs of eGFP-transgenic mice. Red blood cells were lysed with ACK lysis buffer. Monocytes were isolated using a negative selection kit (Miltenyi Biotec, 130-100-629). Cells were washed once with 1X PBS and resuspended in the nucleofection solutions for primary cells (Primary Cell Optimization 96-well Nucleofector Kit, cat# V4SP-9096, Lonza) at a density of 2×10⁵ cells per well. Cells were electroporated as above and immediately transferred to 6-well TC-treated plates containing pre-warmed BMDM culture media. Cells were cultured for 5 days with complete media changes at 2 and 4 days following electroporation. On day 5, monocyte-derived macrophages (Mo-Macs) were harvested by gently scraping them with a rubber policeman. Harvested cells were processed for flow cytometry. First, cells were stained with an Fc-Blocking reagent (CD16/32 FcR block, BD Biosciences) in the presence of a fixable viability dye (eFluor 780) for 20 min at 4C in 1x PBS. Cells were washed twice in flow cytometry buffer, followed by staining for F4/80 (anti-F4/80-BV421). Cells were washed twice and flow cytometry was performed to compare each electroporation condition using a BD Fortessa analyzer. Loss of eGFP in the F4/80-positive population, along with maintenance of cell viability, shown as a lack of APC-Cy7-positive signal, was used to rank each condition.

### Murine BMDC CRISPR KO

Bone marrow (BM) cells were prepared and red blood cells were lysed with ACK lysis buffer. BM cells were washed twice with 1X PBS and electroporated in the appropriate primary nucleofection solution (Primary Cell Optimization 4D-NucleofectorTM X Kit, cat# V4XP-9096, P3 Primary Cell 4D-Nucleofector X Kit, cat# V4XP-3032) using the Lonza 4D Nucleofector (4D-Nucleofector Core Unit: Lonza, AAF-1002B; 4D-Nucleofector X Unit: AAF-1002X) as described above. Specifically, 2×10⁶ BM cells per reaction were resuspended in 20 µl of primary nucleofection solution and mixed with Cas9-RNP containing targeting or NTC gRNAs. The cell/Cas9-RNP mix was then electroporated with the appropriate program. Electroporated cells were cultured in pre-warmed RPMI media supplemented with 10 % FBS, L-Glutamate, HEPES, antibiotics, and 2-ME and 100 ng/ml Flt3-ligand (Peprotech) for 12 days in round bottom 96-well TC treated plates.

### Flow Cytometry Analysis for Murine BMDMs

Harvested BMDCs were stained with fixable viability dye in 1X PBS for 20 min. The centrifuged cell pellets were pre-incubated with FACS buffer containing CD16/32 Fc-block for 10 min. Cells were incubated for additional 30 min with fluorophore-conjugated antibodies in FACS buffer. Cells were washed and fixed with 2 % PFA for 20 min before running flow cytometry. All staining was performed on ice. Subsets of cells in the BMDC cultures were gated as shown in FIGS. 1D, 6A. For intracellular staining of TLR7, BD fix and perm kit was used as instructed. All samples were analyzed by the FACS Fortessa system (BD).

### TLR3, TLR4 and STING Stimulation of Murine BMDMs

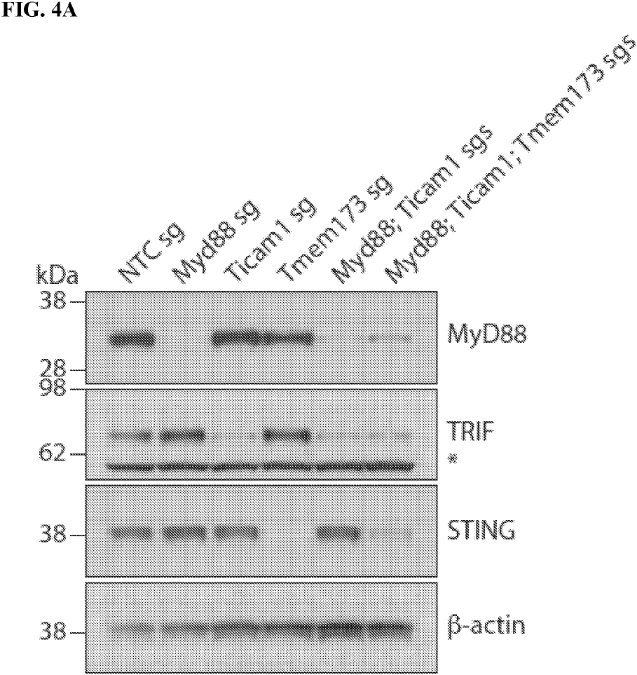

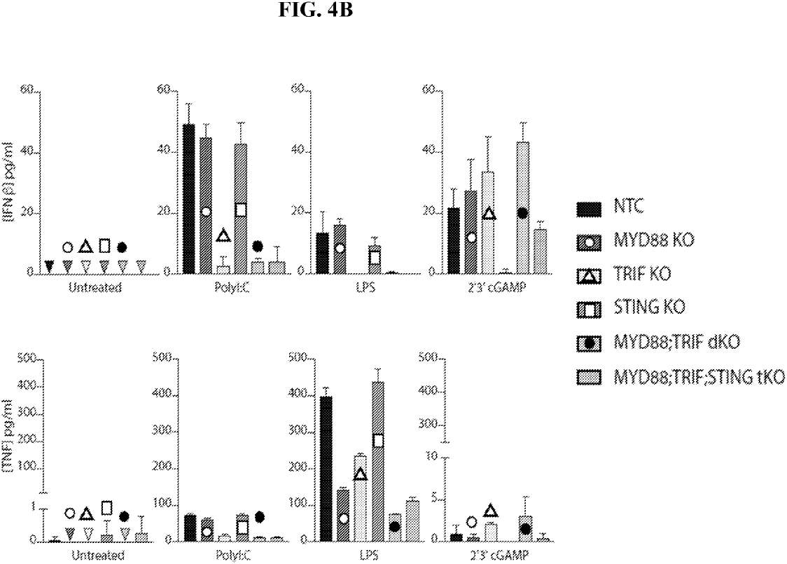

CRISPR KO was performed by electroporating day 5 wild-type BMDMs with non-targeting sgRNA (NTC) or a pool of two sgRNAs targeting MYD88, TRIF (encoded by Ticam1) or STING (encoded by Tmem173) either in isolation or combination as shown in FIG. 4. 5×10⁶ BMDMs were used per reaction (Buffer P3, program CM-137). Immediately after electroporation, cells were transferred to 10 cm non-TC treated Petri dishes containing 10 mL of pre-warmed BMDM culture media. Cells were cultured for an additional 5 days with media changes at day 2 and 4 following electroporation. On day 5, cells were gently scraped and replated in TC-treated multi-well plates at a density of 0.5×10⁶ cells per mL for stimulation. BMDMs were stimulated overnight (18 hr) with 100 ng/mL ultra-pure LPS, 10 ug/mL PolyI:C LMW or 5 ug/mL 2′3′-cGAMP (InvivoGen) to activate TLR4, TLR3 or STING, respectively. Media was collected following treatment for TNF and IFNβ measurements by ELISA. Gene deletion was assessed by Western blot.

### Cas9 Editing Validation by Sequencing and TIDE (Tracking of Indels by DEcomposition)

Primer design. DNA primers were generated for each locus being assessed for editing efficiency. Briefly, NCBI Gene was used to search for the RefSeq of gene of interest. FASTA genomic sequence was searched for the guide sequence and cut site after GG PAM at 2 and 3 basepairs from one end Starting at 350 bp upstream from the PAM, 700 bp of the sequence was selected with the cut site in the middle. This was pasted into Primer 3. “350,2” was entered in the Targets box, “500-600” in the Product Size Ranges box and “2” in the CG Clamp box. “Pick Primers” was clicked and a result selected that contained the cut-site in the middle. Primers were ordered from IDT.

Genomic DNA (gDNA). gDNA was harvested from cells using the QuickExtract solution: ~30 µL Quickextract solution was added to cells for each well of a 96-well plate or 100 µL per well a 24-well and incubated for 1-5 min at room temperature while ensuring cells were detached. Cells were lifted with repeated pipetting and transferred to PCR tubes. Samples were vortexed briefly, incubated at 65° C. for 6 min, then 98° C. for 2 min. gDNA templates were then used for PCR.

PCR. PCR was run with Terra polymerase 35 cycles using 1 µl of gDNA per 20 µL reaction. One test (edited gDNA) reaction and one control (unedited gDNA) reaction was run for each sgRNA target editing site. Setup: 10 µL 2x Buffer, 1 µL gDNA, 1.2 µL Primer mix (containing 1 µM of F and 1 µM R validation primer), 0.4 µL Terra Polymerase, 7.4 µL H2O. 35 cycles were run following the Terra PCR protocol (98C 2 min, [98° C. 10 s, 58° C. 15 s, 68° C. 60 s]×36, 68° C. 5 min). eGel was used to check for ~500 bp product (running just the control unedited reaction for each new primer pair is sufficient). 2 µL was run on a 2% gel, 14 min. PCR reaction cleanup was performed using DNAClean (Zymo Research) or Qiagen columns.

Sequencing and TIDE. Clean PCR product was submitted for Sanger sequencing using the F primer 250 bp upstream of the target editing site. Sanger results were analyzed using TIDE or ICE websites: www.deskgen.com/landing/tide.html or ice.synthego.com/#/.

### Antibodies

Murine. I-A/I-E-BV421, B220-BV605, F4/80-BV711, Sirpα-PE-Cy7, CD45-FITC (Biolegend),TLR7-PE, CD24-BUV395, CD80-BUV747, F4/80-BV421, CD11b-APC (BD)
eBioscience™ Fixable Viability Dye eFluor™ 780 (Thermofisher cat# 65-0865-14)
Western blotting antibodies: TRIF (Genentech, Cat#1-3-5), MYD88 (Abcam, Cat# ab2064), STING (Cell Signaling Technologies D2P2F, Cat# 13647S), beta-actin (Cell Signaling Technologies Cat# 3700).

Human. CD64 Mouse anti-Human, APC (BD Biosciences 561189), FITC anti-human CD163 Antibody (Biolegend 333617), PE anti-human β2-microglobulin Antibody (Biolegend 316305), APC anti-human CD209 (DC-SIGN) Antibody (Biolegend 330107), BV786 Mouse Anti-Human CD80 (BD Biosciences 564159), Alexa Fluor 488 (ThermoFisher cat # A-11008). LIVE/DEAD Fixable Aqua Dead Cell dye (ThermoFisher cat# L34957). LIVE/DEAD™ Fixable Blue Dead Cell Stain Kit, for UV excitation (ThermoFisher cat #L34961).

Western blotting antibodies. Anti-MAVS antibody (Abcam ab31334), Goat anti-Rabbit IgG (H+L) Cross-Adsorbed Secondary Antibody (ThermoFisher, cat# 31212), Goat anti-Mouse IgG (H+L) Cross-Adsorbed Secondary Antibody (ThermoFisher, cat# 31164), PKR (Cell Signaling, CST 12297), eIF2a-P (Cell Signaling, CST 3398), eIF2α -total (Abcam., ab5369), β-Tubulin (Santa Cruz, sc-5274)

### Elisa

Measurement of TNF and IFNβ secretion by murine BMDMs was performed by standard ELISA using commercial kits (TNF, Invitrogen 88-7324-88; IFNβ, PBL Assay Sci., Verikine 422400-1).

### Statistical Analysis

Where depicted, pairwise statistical analyses were performed using an unpaired Student’s two-sided t-test. Scatterplot and bar graphs reflect means of data. GraphPad Prism was used for data analysis and representation.

TABLE 3

Guide RNA (gRNA) sequences

| Description | Sequence | SEQ ID NO: |
|---|---|---|
| Human | | |
| B2M gRNA1 | AAGTCAACTTCAATGTCGGA | 1 |
| B2M gRNA2 | CGTGAGTAAACCTGAATCTT | 2 |
| B2M gRNA3 | ACTCACGCTGGATAGCCTCC | 3 |
| B2M gRNA4 | GAGTAGCGCGAGCACAGCTA | 4 |
| MAVS sg1 | GTAGATACAACTGACCCTGT | 5 |
| MAVS sg2 | TACTAGCATGGTGCTCACCA | 6 |
| PKR sgRNA | CAGGACCTCCACATGATAGG | 7 |
| Mouse | | |
| CD11b/Itgam gRNA1 | TGCAGTACTCGGACGAGTTC | 8 |
| CD11b/Itgam gRNA2 | TTATAAGGATGTCATCCCCG | 9 |
| eGFP gRNA1 | GGTGGTGCAGATGAACTTCA | 10 |
| eGFP gRNA2 | GGAGCGCACCATCTTCTTCA | 11 |
| eGFP gRNA3 | GGCATCGACTTCAAGGAGGA | 12 |
| CD45 gRNA | AAACGCCTAAGCCTAGTTGT | 13 |
| TLR7 sg1 | TGTGCAGTCCACGATCACAT | 14 |
| TLR7 sg2 | ATCGAGGGCAATTTCCACTT | 15 |
| TRIF/Ticam1 sg1 | TCTGGTGTGTCAATGGGACG | 16 |
| TRIF/Ticam1 sg2 | CAAGCTATGTAACACACCGC | 17 |
| MYD88 sg1 | CCCACGTTAAGCGCGACCAA | 18 |
| MYD88 sg2 | GTCTGCGGGAGACCCCCGCG | 19 |
| STING/Tmem173 sg1 | CAGTAGTCCAAGTTCGTGCG | 20 |
| STING/Tmem173 sg2 | CACCTAGCCTCGCACGAACT | 21 |
| NTC gRNA (luciferase) | GCATGCGAGAATCTCACGCA | 22 |

TABLE 4

Primer sequences to validate human MAVS Cas9 editing

| | gRNA sequence | Forward Primer | Reverse Primer | ICE score |
|---|---|---|---|---|
| hMAVS_AC | GTAGATACAACTGA CCCTGT (SEQ ID NO:23) | CAGGAAGCAGTGACCA AAGG (SEQ ID NO:24) | TTTGGATGGTGCTGGAT TGG (SEQ ID NO:25) | 63, 65 |
| hMAVS_8 | TACTAGCATGGTGC TCACCA (SEQ ID NO:26) | GCTGCAGAGGGTAAAC AGGG (SEQ ID NO:27) | TCCTGGAGAACATGGTG TGG (SEQ ID NO:28) | 89, 90 |

### REFERENCES

Ablasser, A., M. Goldeck, T. Cavlar, T. Deimling, G. Witte, I. Rohl, K.P. Hopfner, J. Ludwig, and V. Hornung. 2013. cGAS produces a 2′-5′-linked cyclic dinucleotide second messenger that activates STING. Nature 498:380-384.

Baer, C., M.L. Squadrito, D. Laoui, D. Thompson, S.K. Hansen, A. Kiialainen, S. Hoves, C.H. Ries, C.H. Ooi, and M. De Palma. 2016. Suppression of microRNA activity amplifies IFN-gamma-induced macrophage activation and promotes anti-tumour immunity. Nat Cell Biol 18:790-802.

Baker, P.J., and S.L. Masters. 2018. Generation of Genetic Knockouts in Myeloid Cell Lines Using a Lentiviral CRISPR/Cas9 System. Methods Mol Biol 1714:41-55.

Bassler, K., J. Schulte-Schrepping, S. Warnat-Herresthal, A.C. Aschenbrenner, and J.L. Schultze. 2019. The Myeloid Cell Compartment-Cell by Cell. Annu Rev Immunol 37:269-293.

Bobadilla, S., N. Sunseri, and N.R. Landau. 2013. Efficient transduction of myeloid cells by an HIV-1-derived lentiviral vector that packages the Vpx accessory protein. Gene Ther 20:514-520.

Bowles, R., S. Patil, H. Pincas, and S.C. Sealfon. 2011. Optimized protocol for efficient transfection of dendritic cells without cell maturation. J Vis Exp e2766.

Broz, M.L., M. Binnewies, B. Boldajipour, A.E. Nelson, J.L. Pollack, D.J. Erle, A. Barczak, M.D. Rosenblum, A. Daud, D.L. Barber, S. Amigorena, L.J. Van’t Veer, A.I. Sperling, D.M. Wolf, and M.F. Krummel. 2014. Dissecting the tumor myeloid compartment reveals rare activating antigen-presenting cells critical for T cell immunity. Cancer Cell 26:638-652.

Chen, J., Y. Du, X. He, X. Huang, and Y.S. Shi. 2017. A Convenient Cas9-based Conditional Knockout Strategy for Simultaneously Targeting Multiple Genes in Mouse. Sci Rep 7:517.

Coch, C., C. Luck, A. Schwickart, B. Putschli, M. Renn, T. Holler, W. Barchet, G. Hartmann, and M. Schlee. 2013. A human in vitro whole blood assay to predict the systemic cytokine response to therapeutic oligonucleotides including siRNA. PLoS One 8:e71057.

Cornel, A.M., N.P. van Til, J.J. Boelens, and S. Nierkens. 2018. Strategies to Genetically Modulate Dendritic Cells to Potentiate Anti-Tumor Responses in Hematologic Malignancies. Front Immunol 9:982.

Dabo, S., and E.F. Meurs. 2012. dsRNA-dependent protein kinase PKR and its role in stress, signaling and HCV infection. Viruses 4:2598-2635.

Doench, J.G. 2018. Am I ready for CRISPR? A user’s guide to genetic screens. Nat Rev Genet 19:67-80.

Edwards, D.K.t., K. Watanabe-Smith, A. Rofelty, A. Damnernsawad, T. Laderas, A. Lamble, E.F. Lind, A. Kaempf, M. Mori, M. Rosenberg, A. d′Almeida, N. Long, A. Agarwal, D.T. Sweeney, M. Loriaux, S.K. McWeeney, and J.W. Tyner. 2019. CSF1R inhibitors exhibit antitumor activity in acute myeloid leukemia by blocking paracrine signals from support cells. Blood 133:588-599.

Gay, N.J., M.F. Symmons, M. Gangloff, and C.E. Bryant. 2014. Assembly and localization of Toll-like receptor signalling complexes. Nat Rev Immunol 14:546-558.

Geissmann, F., M.G. Manz, S. Jung, M.H. Sieweke, M. Merad, and K. Ley. 2010. Development of monocytes, macrophages, and dendritic cells. Science 327:656-661.

Geissmann, F., and E. Mass. 2015. A stratified myeloid system, the challenge of understanding macrophage diversity. Semin Immunol 27:353-356.

Ginhoux, F., and M. Guilliams. 2016. Tissue-Resident Macrophage Ontogeny and Homeostasis. Immunity 44:439-449.

Gundry, M.C., L. Brunetti, A. Lin, A.E. Mayle, A. Kitano, D. Wagner, J.I. Hsu, K.A. Hoegenauer, C.M. Rooney, M.A. Goodell, and D. Nakada. 2016. Highly Efficient Genome Editing of Murine and Human Hematopoietic Progenitor Cells by CRISPR/Cas9. Cell Rep 17:1453-1461.

Hammerschmidt, S.I., K. Werth, M. Rothe, M. Galla, M. Permanyer, G.E. Patzer, A. Bubke, D.N. Frenk, A. Selich, L. Lange, A. Schambach, B. Bosnjak, and R. Forster. 2018. CRISPR/Cas9 Immunoengineering of Hoxb8-Immortalized Progenitor Cells for Revealing CCR7-Mediated Dendritic Cell Signaling and Migration Mechanisms in vivo. Front Immunol 9:1949.

Hendel, A., R.O. Bak, J.T. Clark, A.B. Kennedy, D.E. Ryan, S. Roy, I. Steinfeld, B.D. Lunstad, R.J. Kaiser, A.B. Wilkens, R. Bacchetta, A. Tsalenko, D. Dellinger, L. Bruhn, and M.H. Porteus. 2015. Chemically modified guide RNAs enhance CRISPR-Cas genome editing in human primary cells. Nat Biotechnol 33:985-989.

Hornung, V., J. Ellegast, S. Kim, K. Brzozka, A. Jung, H. Kato, H. Poeck, S. Akira, K.K. Conzelmann, M. Schlee, S. Endres, and G. Hartmann. 2006. 5′-Triphosphate RNA is the ligand for RIG-I. Science 314:994-997.

Jakubzick, C.V., G.J. Randolph, and P.M. Henson. 2017. Monocyte differentiation and antigen-presenting functions. Nat Rev Immunol 17:349-362.

Kim, S., T. Koo, H.G. Jee, H.Y. Cho, G. Lee, D.G. Lim, H.S. Shin, and J.S. Kim. 2018. CRISPR RNAs trigger innate immune responses in human cells. Genome Res

Kuhn, S., E.J. Hyde, J. Yang, F.J. Rich, J.L. Harper, J.R. Kirman, and F. Ronchese. 2013. Increased numbers of monocyte-derived dendritic cells during successful tumor immunotherapy with immune-activating agents. J Immunol 191:1984-1992.

Kumar, V., L. Donthireddy, D. Marvel, T. Condamine, F. Wang, S. Lavilla-Alonso, A. Hashimoto, P. Vonteddu, R. Behera, M.A. Goins, C. Mulligan, B. Nam, N. Hockstein, F. Denstman, S. Shakamuri, D.W. Speicher, A.T. Weeraratna, T. Chao, R.H. Vonderheide, L.R. Languino, P. Ordentlich, Q. Liu, X. Xu, A. Lo, E. Pure, C. Zhang, A. Loboda, M.A. Sepulveda, L.A. Snyder, and D.I. Gabrilovich. 2017. Cancer-Associated Fibroblasts Neutralize the Anti-tumor Effect of CSF1 Receptor Blockade by Inducing PMN-MDSC Infiltration of Tumors. Cancer Cell 32:654-668 e655.

Leyva, F.J., J.J. Anzinger, J.P. McCoy, Jr., and H.S. Kruth. 2011. Evaluation of transduction efficiency in macrophage colony-stimulating factor differentiated human macrophages using HIV-1 based lentiviral vectors. BMC Biotechnol 11:13.

Li, T., and Z.J. Chen. 2018. The cGAS-cGAMP-STING pathway connects DNA damage to inflammation, senescence, and cancer. J Exp Med 215:1287-1299.

Lim, J., H. Park, J. Heisler, T. Maculins, M. Roose-Girma, M. Xu, B. McKenzie, M. van Lookeren Campagne, K. Newton, and A. Murthy. 2019. Autophagy regulates inflammatory programmed cell death via turnover of RHIM-domain proteins. Elife 8:

Merad, M., P. Sathe, J. Helft, J. Miller, and A. Mortha. 2013. The dendritic cell lineage: ontogeny and function of dendritic cells and their subsets in the steady state and the inflamed setting. Annu Rev Immunol 31:563-604.

Meurs, E.F., Y. Watanabe, S. Kadereit, G.N. Barber, M.G. Katze, K. Chong, B.R. Williams, and A.G. Hovanessian. 1992. Constitutive expression of human double-stranded RNA-activated p68 kinase in murine cells mediates phosphorylation of eukaryotic initiation factor 2 and partial resistance to encephalomyocarditis virus growth. J Virol 66:5805-5814.

Naik, S.H., A.I. Proietto, N.S. Wilson, A. Dakic, P. Schnorrer, M. Fuchsberger, M.H. Lahoud, M. O’Keeffe, Q.X. Shao, W.F. Chen, J.A. Villadangos, K. Shortman, and L. Wu. 2005. Cutting edge: generation of splenic CD8+ and CD8- dendritic cell equivalents in Fms-like tyrosine kinase 3 ligand bone marrow cultures. J Immunol 174:6592-6597.

Napier, B.A., S.W. Brubaker, T.E. Sweeney, P. Monette, G.H. Rothmeier, N.A. Gertsvolf, A. Puschnik, J.E. Carette, P. Khatri, and D.M. Monack. 2016. Complement pathway amplifies caspase-11-dependent cell death and endotoxin-induced sepsis severity. J Exp Med 213:2365-2382.

Parnas, O., M. Jovanovic, T.M. Eisenhaure, R.H. Herbst, A. Dixit, C.J. Ye, D. Przybylski, R.J. Platt, I. Tirosh, N.E. Sanjana, O. Shalem, R. Satija, R. Raychowdhury, P. Mertins, S.A. Carr, F. Zhang, N. Hacohen, and A. Regev. 2015. A Genome-wide CRISPR Screen in Primary Immune Cells to Dissect Regulatory Networks. Cell 162:675-686.

Perry, C.J., A.R. Munoz-Rojas, K.M. Meeth, L.N. Kellman, R.A. Amezquita, D. Thakral, V.Y. Du, J.X. Wang, W. Damsky, A.L. Kuhlmann, J.W. Sher, M. Bosenberg, K. Miller-Jensen, and S.M. Kaech. 2018. Myeloid-targeted immunotherapies act in synergy to induce inflammation and antitumor immunity. J Exp Med 215:877-893.

Platt, R.J., S. Chen, Y. Zhou, M.J. Yim, L. Swiech, H.R. Kempton, J.E. Dahlman, O. Parnas, T.M. Eisenhaure, M. Jovanovic, D.B. Graham, S. Jhunjhunwala, M. Heidenreich, R.J. Xavier, R. Langer, D.G. Anderson, N. Hacohen, A. Regev, G. Feng, P.A. Sharp, and F. Zhang. 2014. CRISPR-Cas9 knockin mice for genome editing and cancer modeling. Cell 159:440-455.

Roberts, A.W., L.M. Popov, G. Mitchell, K.L. Ching, D.J. Licht, G. Golovkine, G.M. Barton, and J.S. Cox. 2019. Cas9(+) conditionally-immortalized macrophages as a tool for bacterial pathogenesis and beyond. Elife 8:

Ryan, D.E., D. Taussig, I. Steinfeld, S.M. Phadnis, B.D. Lunstad, M. Singh, X. Vuong, K.D. Okochi, R. McCaffrey, M. Olesiak, S. Roy, C.W. Yung, B. Curry, J.R. Sampson, L. Bruhn, and D.J. Dellinger. 2018. Improving CRISPR-Cas specificity with chemical modifications in single-guide RNAs. Nucleic Acids Res 46:792-803.

Seki, A., and S. Rutz. 2018. Optimized RNP transfection for highly efficient CRISPR/Cas9-mediated gene knockout in primary T cells. J Exp Med 215:985-997.

Seth, R.B., L. Sun, C.K. Ea, and Z.J. Chen. 2005. Identification and characterization of MAVS, a mitochondrial antiviral signaling protein that activates NF-kappaB and IRF 3. Cell 122:669-682.

Simeonov, D.R., and A. Marson. 2019. CRISPR-Based Tools in Immunity. Annu Rev Immunol 37:571-597.

Stromnes, I.M., A.L. Burrack, A. Hulbert, P. Bonson, C. Black, J.S. Brockenbrough, J.F. Raynor, E.J. Spartz, R.H. Pierce, P.D. Greenberg, and S.R. Hingorani. 2019. Differential Effects of Depleting versus Programming Tumor-Associated Macrophages on Engineered T Cells in Pancreatic Ductal Adenocarcinoma. Cancer Immunol Res 7:977-989.

Ting, P.Y., A.E. Parker, J.S. Lee, C. Trussell, O. Sharif, F. Luna, G. Federe, S.W. Barnes, J.R. Walker, J. Vance, M.Y. Gao, H.E. Klock, S. Clarkson, C. Russ, L.J. Miraglia, M.P. Cooke, A.E. Boitano, P. McNamara, J. Lamb, C. Schmedt, and J.L. Snead. 2018. Guide Swap enables genome-scale pooled CRISPR-Cas9 screening in human primary cells. Nat Methods 15:941-946.

Varol, C., A. Mildner, and S. Jung. 2015. Macrophages: development and tissue specialization. Annu Rev Immunol 33:643-675.

Wienert, B., J. Shin, E. Zelin, K. Pestal, and J.E. Corn. 2018. In vitro-transcribed guide RNAs trigger an innate immune response via the RIG-I pathway. PLoS Biol 16:e2005840.

Wynn, T.A., and K.M. Vannella. 2016. Macrophages in Tissue Repair, Regeneration, and Fibrosis. Immunity 44:450-462.

1. A method for genetic modification of a myeloid cell, the method comprising transfecting the myeloid cell with a gene editing reagent targeting a genetic site of interest, wherein the myeloid cell is not transduced with a viral vector.

2. The method of claim 1, wherein the myeloid cell is a primary myeloid cell.

3. The method of claim 1, wherein the myeloid cell is a monocyte, macrophage, neutrophil, basophil, eosinophil, erythrocyte, dendritic cell, or megakaryocyte.

4. The method of claim 1, wherein the cell is transfected via electroporation, nucleofection, lipid-based transfection, or polymer-based transfection.

5-7. (canceled)

8. The method of claim 1, wherein the cell is not subjected to a selection step and/or an enrichment step after transfection.

9. The method of claim 1, wherein the gene editing reagent comprises an RNA-guided nuclease.

10-12. (canceled)

13. The method of claim 1, wherein the gene editing reagent comprises a CRISPR-Cas system comprising a Cas protein, a guide RNA, and optionally a donor DNA.

14-15. (canceled)

16. The method of claim 1, wherein the gene editing reagent comprises transcription activator-like effector nuclease (TALEN), a zinc finger nuclease, or an Argonaut endonuclease.

17. The method of claim 1, wherein the myeloid cell is contacted with an electroporation enhancer during transfection.

18. The method of claim 17, wherein the electroporation enhancer is selected from a carrier DNA, a single stranded DNA, a combination of single stranded and double stranded DNA, a polymeric additive, and an oligonucleotide.

19. The method of claim 18, wherein the carrier DNA is a single-stranded DNA oligonucleotide.

20. The method of claim 18, wherein the carrier DNA is nonhomologous to human, mouse, and/or rat genomes.

21. The method of claim 1, wherein the myeloid cell is differentiated prior to electroporation.

22. The method of claim 21, wherein the myeloid cell is differentiated into a dendritic cell.

23. (canceled)

24. The method of claim 1, wherein the myeloid cell is not activated prior to or during genetic modification.

25. The method of claim 1, wherein two or more distinct crispr RNAs (crRNA) to the site of interest are introduced into the myeloid cell.

26-28. (canceled)

29. The method of claim 25, wherein the crRNAs are single guide RNAs (sgRNAs).

30. The method of claim 1, wherein multiple genetic sites of interest are targeted.

31-32. (canceled)

33. A method for genetic modification of a plurality of myeloid cells, the method comprising transfecting the plurality of myeloid cells in the presence of a gene editing reagent targeting a site of interest, wherein the myeloid cells are not transduced with a viral vector.

34-36. (canceled)

37. The method of claim 33, wherein the site of interest is modified in at least 70% of the plurality of myeloid cells.

38. The method of claim 37, wherein the site of interest is modified in at least 80% of the plurality of myeloid cells.

39. The method of claim 37, wherein the site of interest is modified in at least 85% of the plurality of myeloid cells.

40. The method of claim 37, wherein the site of interest is modified in at least 90% of the plurality of myeloid cells.

41. The method of claim 33, wherein the plurality of myeloid cells comprises dendritic cells (DCs) and the site of interest is modified in at least 50% of the DCs.

42. The method of claim 33, wherein the viability of the plurality of myeloid cells after electroporation is at least 80%.

43. The method of claim 42, wherein the viability of the plurality of myeloid cells after electroporation is at least 90%.

44-51. (canceled)

52. The method of claim 9, wherein the RNA-guided nuclease comprises a guide RNA and a ribonucleoprotein (RNP), wherein the ratio of guide RNA to RNP is between 100:1 and 1:100.

53. The method of claim 52, wherein the ratio of guide RNA to RNP is less than or equal to about 3:1.

54. The method of claim 52, wherein the ratio of guide RNA to ribonucleoprotein (RNP) is less than or equal to about 2:1.

55. The method of claim 52, wherein the ratio of guide RNA to ribonucleoprotein (RNP) is about 3:1.

56. The method of claim 52, wherein the ratio of guide RNA to ribonucleoprotein (RNP) is about 2:1.

57. The method of claim 33, wherein at least 70% of the transfected cells are administered to a patient in need thereof, without selection or enrichment of the cells.

58. (canceled)

59. A method of genetically modifying a plurality of myeloid cells, comprising transfecting the myeloid cells with a gene editing reagent, wherein the myeloid cells are not transduced with a viral vector, and wherein the method does not comprise a selection step or enrichment step following myeloid cell transfection.

60-65. (canceled)

66. The method of claim 59, wherein the plurality of myeloid cells comprises dendritic cells (DCs) and the site of interest is modified in at least 50% of the DCs.

67-76. (canceled)

77. A system for genetically modifying a myeloid cell in the absence of a viral vector, the system comprising a chamber compatible with a transfection system, multiple myeloid cells within the chamber in a media compatible with electroporation, and at least one gene editing system designed to target at least one site of interest in the genome of myeloid cells.

78-96. (canceled)

97. A method of treating a disease treatable with a myeloid cell, comprising providing a genetically modified myeloid cell that has not been transduced with a virus, wherein the myeloid cell has been transfected with a gene editing reagent; and administering the myeloid cell to a patient in need thereof.

98-108. (canceled)

109. A composition comprising a plurality of myeloid cells, a gene editing reagent, a transfection buffer, and an electroporation enhancer, wherein the composition does not comprise a viral vector.

110-126. (canceled)

127. A genetically modified myeloid cell made by the method of claim 1 .

128. An assay for drug discovery comprising screening the effect of one or more compounds on the myeloid cell of claim 127.

129. A method for target validation of a compound, comprising contacting a myeloid cell of claim 127 with the compound and monitoring an effect on the cell.
